(19) United States
(12) Patent Application Publication
(10) Pub. No.: US 2022/0031800 A1
(43) Pub. Date: Feb. 3, 2022

(54) PEPTIDES FOR TREATING NON-EXUDATIVE MACULAR DEGENERATION AND OTHER DISORDERS OF THE EYE

(71) Applicant: Allegro Pharmaceuticals, LLC, San Juan Capistrano, CA (US)

(72) Inventors: Hampar L. Karageozian, San Juan Capistrano, CA (US); John Y. Park, Santa Ana, CA (US); Vicken H. Karageozian, San Juan Capistrano, CA (US)

(21) Appl. No.: 16/938,758

(22) Filed: Jul. 24, 2020

Related U.S. Application Data
(60) Provisional application No. 62/879,281, filed on Jul. 26, 2019.

(51) Int. Cl. A61K 38/08; A61P 27/02

(52) U.S. Cl. CPC A61K 38/08; A61P 27/02

(57) ABSTRACT

Methods of using anti-integrin peptides for a) improving best corrected visual acuity of an eye of a subject suffering from non-exudative age related macular degeneration and/or b) improving color vision in an eye of a subject suffering from impaired color vision and/or for treatment of other disorders.

### RELATED APPLICATION

This patent application claims priority to U.S. Provisional Patent Application No. 62/879,281 entitled Peptides for Treating Dry Macular Degeneration and Other Disorders of the Eye filed Jul. 26, 2019, the entire disclosure of which is expressly incorporated herein.

### FIELD OF THE INVENTION

The present disclosure relates generally to the fields of chemistry, life sciences, pharmacy and medicine and more particularly to pharmaceutical preparations and their use in the treatment of eye disorders.

### BACKGROUND

Pursuant to 37 CFR 1.71(e), this patent document contains material which is subject to copyright protection and the owner of this patent document reserves all copyright rights whatsoever.

Throughout this patent application, ranges may be specified as “Value 1 to Value 2.” Unless otherwise specified, the use of the word “to” in this context is shall be interpreted as being inclusive of the stated upper and lower values defining the range. Thus, unless otherwise specified, a range defined as extending from Value 1 ‘to” Value 2 shall be interpreted as being inclusive of Value 1, Value 2 and all values therebetween.

Also, throughout this patent application amino acids may be referred to interchangeably using the names, three letter codes and/or single letter codes set forth in the following table:

| Amino Acid | Three letter code | Single Letter Code |
|---|---|---|
| Alanine | Ala | A |
| Arginine | Arg | R |
| Asparagine | Asn | N |
| Aspartic Acid | Asp | D |
| Cysteine | Cys | C |
| Cysteic Acid | Cys(Acid) | — |
| Glutamic | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tyrosine | Tyr | Y |
| Valine | Val | V |

Applicant is developing Risuteganib, a non-natural peptide having the molecular formula C22-H39-N9-O11-S and the following structural formula:

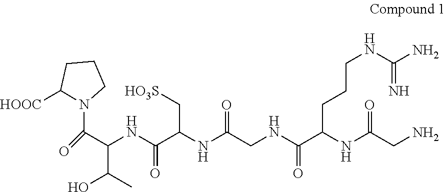 [SMILES: CC(O)C(NC(=O)C(CS(=O)(=O)O)NC(=O)CNC(=O)C(CCCNC(=N)N)NC(=O)CN)C(=O)N1CCCC1C(=O)O]

Risuteganib and preparations containing risuteganib have also been referred to by other names, nomenclatures and designations, including: risuteganib; Glycyl-L-arginylglycyl-3-sulfo-L-alanyl-L-threonyl-L-proline; Arg-Gly-NH—CH(CH₂—SO₃H)COOH; ALG-1001 and Luminate® (Allegro Ophthalmics, LLC, San Juan Capistrano, Calif.).

Risuteganib is an anti-integrin peptide, which inhibits a number of integrins upstream in the oxidative stress pathway. Risuteganib acts broadly to downregulate multiple angiogenic and inflammatory processes, including those associated with hypoxia and oxidative stress.

Additional description of and information relating to Risuteganib is provided in U.S. Pat. Nos. 9,018,352; 9,872,886; 9,896,480 and 10,307,460 and in United States Patent Application Publication Nos. 2018/0207227 and 2019/0062371, the entire disclosure of each such patent and patent application being expressly incorporated herein by reference. There are two basic types of age related macular degeneration: non-exudative or “dry” and exudative or “wet.” In contrast to the exudative or “wet” form of the disease, non-exudative age related macular degeneration (referred to below as “Dry AMD”) does not involve leakage of blood or serum from small blood vessels of the retina. In some patients, Dry AMD may progress to Wet AMD. Patients who suffer from Dry AMD typically experience progressive loss of visual acuity due to thinning of the macula, which is a central part of the retina.

In Dry AMD, deposits of amorphous yellow debris known as drusen typically form adjacent to the basement membrane of the retinal pigment epithelium. This leads to thinning and desiccation of the macula, which in turn results in loss of central visual acuity. Patients who suffer from Dry AMD typically experience progressive loss of visual acuity due to thinning of the macula, which is a central part of the retina.

In the past, there has been no known cure for Dry AMD. Treatments for Dry AMD have typically include the use of nutritional supplements recommended by the Age-Related Eye Disease Study 2 (AREDS2) as well as controlling diet, weight, blood pressure and smoking, and exposure to blue and ultraviolet light. While these treatment modalities may slow the progression of Dry AMD, they are not recognized as being effective to actually reverse loss of vision that has already occurred due to Dry AMD.

Risuteganib was previously believed to have utility in treating age related macular degeneration by reducing inflammation and deterring the onset of pathological neovascularization, which is a hallmark of the progression of Dry (non-exudative) AMD to Wet (exudative) AMD.

As disclosed herein, Applicant has generated date indicating that risuteganib administration to subjects suffering from Dry AMD, which has not progressed to Wet AMD, may not only reduce inflammation and delay potential onset of pathological neovascularization, but also provide measurable improvements in visual acuity and optical anatomy.

### SUMMARY OF THE DISCLOSURE

The present disclosure describes methods and compositions for treating disorders of the eye and for improving best corrected visual acuity in subjects suffering from Dry AMD and/or improving color vision in subjects suffering from impaired color vision.

In accordance with one aspect of the present disclosure, there are provided methods for a) improving best corrected visual acuity of an eye of a subject suffering from non-exudative age related macular degeneration and/or b) improving color vision in an eye of a subject suffering from impaired color vision, said method comprising the step of administering to the subject an anti-integrin peptide in an amount which is effective to improve best corrected visual acuity and/or color vision in said eye.

In some embodiments of the herein-disclosed methods, the peptide is linear or cyclic and comprises Glycinyl-Arginyl-Glycinyl-Cysteic Acid-Threonyl-Proline-COOH or a fragment, congener, derivative, pharmaceutically acceptable salt, hydrate, isomer, multimer, cyclic form, linear form, conjugate, derivative or other modified form thereof.

In some of the herein-disclosed methods, the peptide comprises risuteganib.

In some of the herein-disclosed methods, the peptide may have the formula:

X1-R-G-Cysteic Acid-X
where X and X1 are independently selected from: Phe-Val-Ala, -Phe-Leu-Ala, -Phe-Val-Gly, -Phe-Leu-Gly, -Phe-Pro-Gly, -Phe-Pro-Ala, -Phe-Val; or from Arg, Gly, Cysteic, Phe, Val, Ala, Leu, Pro, Thr and salts, combinations, D-isomers and L-isomers thereof.

In some of the herein-disclosed methods, the peptide may have the formula:

Y—X—Z
wherein: Y=R, H, K, Cys(acid), G or D; X=G, A, Cys(acid), R, G, D or E; and Z=Cys(acid), G, C, R, D, N or E.

In some of the herein-disclosed methods, the peptide may comprise or consist of an amino acid sequence selected from: R-G-Cys(Acid), R—R-Cys, R-CysAcid)-G, Cys(Acid)-R-G, Cys(Acid)-G-R, R-G-D, R-G-Cys(Acid). H-G-Cys(Acid), R-G-N, DG-R, R-D-G, R-A-E, K-G-D, R-G-Cys(Acid)-G-G-G-D-G, Cyclo-{R-G-Cys(acid)-F—N-Me-V}, R-A-Cys (Acid), R-G-C, K-G-D, Cys(acid)-R-G, Cys(Acid)-G-R, Cyclo-{R-G-D-D-F—NMe-V}, H-G-Cys(acid) and salts thereof.

In some of the herein-disclosed methods, the peptide is administered intraviterally, or by any other effective route of administration including but not limited to topical and systemic routes (e.g., eye drops, oral, intravenous, intramuscular, subcutaneous, intranasal, buccal, transdermal, etc.) or by release from a suitable drug delivery implant substance or device.

In some of the herein-disclosed methods, the peptide may comprise risuteganib administered intraviterally at a dose in the range of from 0.01 mg risuteganib to 10.0 mg risuteganib; or at a dose in the range of from 0.05 mg risuteganib to 5.0 mg risuteganib; or at a dose in the range of from 1.0 mg risuteganib to 1.5 mg risuteganib.

In some of the herein described methods, the peptide may be administered only once.

In some of the herein-disclosed methods, the peptide may be administered a plurality of times.

In some of the herein-disclosed methods, the peptide may be administered a plurality of times with an interval of from 1 week to 20 weeks between administrations of the peptide; or with an interval of from 12 weeks to 16 weeks between administrations of the peptide.

In some of the herein-disclosed methods, the peptide comprises risuteganib administered intraviterally one or more times wherein each intravitreal administration delivers a dose of 1 mg. to 1.5 mg risuteganib.

In some of the herein-disclosed methods, the anti-integrin peptide causes downregulation of integrin αMβ2.

In some of the herein-disclosed methods, the anti-integrin peptide reduces expression of a complement 3 receptor.

Further aspects and details of the present disclosure will be understood upon reading of the detailed description and examples set forth herebelow.

### BRIEF DESCRIPTION OF THE DRAWINGS

The following figures are included in this patent application and referenced in the following Detailed Description. These figures are intended only to illustrate certain aspects or embodiments of the present disclosure and do not limit the scope of the present disclosure in any way:

FIG. 1 is a graph showing mean change in BCVA visit in a study of human Subjects suffering from Dry AMD.

FIG. 2A is a graph showing the change in Total Error Score Hue Style by Change in Letters Read from Baseline at Week 12 in Dry AMD eyes after intravitreal injection of 1 mg risuteganib.

FIG. 2B is a graph showing the change in Total Error Score Hue Style by change from baseline in Letters Read at Week 12 in Dry AMD eyes after sham injection.

FIG. 3 is a graph showing change in Total Error Score Hue Style for Risuteganib Responders (at 32 Weeks) Versus Sham Responders (at 12 Weeks).

FIG. 4A is a graph showing change in Mean Retinal Sensitivity by change from baseline in Letters Read in Dry AMD eyes at Week 12 after intravitreal injection of 1 mg risuteganib.

FIG. 4B is a graph showing change in Mean Retinal Sensitivity by change from baseline in Letters Read in Dry AMD eyes at Week 12 after sham injection.

FIG. 5 is a graph showing change in Mean Retinal Sensitivity for Risuteganib Responders (at 32 Weeks) versus Sham Responders (at 12 Weeks).

FIG. 6A is a graph showing change in microperimetry as measured by Number of Loci Summed by Change from Baseline Number of Letters Read in Dry AMD eyes at Week 12 at after intravitreal injection of 1 mg risuteganib.

FIG. 6B is a graph showing change in microperimetry as measured by Number of Loci Summed by Change from Baseline Number of Letters Read in Dry AMD eyes at Week 12 after sham injection.

FIG. 7 is a graph showing change in microperimetry as measured by Number of Loci Summed for Risuteganib Responders (at 32 Weeks) Versus Sham Responders (at 12 Weeks).

FIG. 8A shows locations and incidences of Geographic Atrophy (GA) at baseline (pre-treatment) in Group 1 eyes.

FIG. 8B shows locations and incidences of Geographic Atrophy (GA) at baseline (pre-treatment) in Group 2 eyes.

FIG. 9A shows an external limiting membrane map of the central 1- and 2-mm subfields exhibiting no disruption.

FIG. 9B shows an external limiting membrane map of the central 1- and 2-mm subfields exhibiting segmental disruption.

FIG. 9C shows an external limiting membrane map of the central 1- and 2-mm subfields exhibiting diffuse disruption affecting the fovea.

FIG. 10A shows an OCT image (greyscale) taken from a risuteganib responder eye.

FIG. 10B shows an OCT image (greyscale) taken from a risuteganib responder eye with an overlay of mapping of the individual retinal layers.

FIG. 10C shows an ILM-RPE map of a risuteganib responder eye.

FIG. 10D shows an EZ-RPE map of a risuteganib responder eye.

FIG. 10E shows an RPE-BM map of a risuteganib responder eye.

FIG. 11A shows an OCT image (greyscale) taken from a risuteganib non-responder eye.

FIG. 11B shows an OCT image (greyscale) taken from a risuteganib non-responder eye with an overlay of mapping of the individual retinal layers.

FIG. 11C shows an ILM-RPE map of a risuteganib non-responder eye.

FIG. 11D shows an EZ-RPE map of a risuteganib non-responder eye.

FIG. 11E shows an RPE-BM map of a risuteganib non-responder eye.

FIG. 12A is a bar graph comparing the effects of risuteganib vs. control on gene expression under ITGAM and ITGB2 conditions in retinitis of prematurity (ROP) mice.

FIG. 12B is a bar graph showing the effects of risuteganib vs control on expression of genes associated with complement, cell adhesion and leukocyte migration, in ROP mice.

FIG. 13A is a bar graph showing the effect of risuteganib vs. control on retinal neuronal cell survival following exposure to kainic acid.

FIG. 13B is a bar graph showing the effect of risuteganib vs. control on retinal Muller cell survival following exposure to kainic acid.

FIG. 13 C is a bar graph showing the effect of risuteganib vs. control on retinal pigment epithelium (RPE) cells following exposure to peroxide.

FIG. 14 is a bar graph showing mouse Müller cell viability after cytotoxic stress and risuteganib treatment.

FIG. 15 is a bar graph showing mouse retinal neuron cell viability after cytotoxic stress and risuteganib treatment.

FIG. 16 is a bar graph showing mouse RPE cell viability after cytotoxic stress and risuteganib treatment.

FIG. 17 is a bar graph showing human (MIO-M1) Muller cell viability after risuteganib treatment at three dosage levels vs control.

FIG. 18 is a bar graph showing human (MIO-M1) Muller cell viability after treatment with anti-VEGF agents (Lucentis, Avastin and Eylea) and risuteganib (Luminate) treatments.

FIG. 19 (4-9) is a bar graph showing levels of reactive oxygen species (ROS) in human (MIO-M1) Muller cells after treatment with anti-VEGF agents (Lucentis, Avastin and Eylea) and risuteganib (Luminate) treatments.

FIG. 20 (4-10) is a bar graph showing mitochondrial membrane potential in human (MIO-M1) Muller cells after treatment with anti-VEGF agents (Lucentis, Avastin and Eylea) and risuteganib (Luminate) treatments.

FIG. 21A is a bar graph comparing the effects of control vs. hydroquinone vs hydroquinone+risuteganib on mitochondrial membrane potential in RPE cells.

FIG. 21B is a bar graph comparing the effects of control vs. hydroquinone vs hydroquinone+risuteganib on production of reactive oxygen species (ROS) in RPE cells.

FIG. 21C is a bar graph comparing the effects of control vs. hydroquinone vs hydroquinone+risuteganib on viability of RPE cells.

### DETAILED DESCRIPTION

The following detailed description and the accompanying drawings to which it refers are intended to describe some, but not necessarily all, examples or embodiments of the invention. The described embodiments are to be considered in all respects only as illustrative and not restrictive. The contents of this detailed description and the accompanying drawings do not limit the scope of the invention in any way.

As used herein, the term “patient or “subject” refers to either human or non-human animals, such as humans, primates, mammals, and vertebrates.

As used herein, the term “treat” or “treating” refers to preventing, eliminating, curing, deterring, reducing the severity or reducing at least one symptom of a condition, disease or disorder.

As used herein, the phrase “effective amount” or “amount effective to” refers to an amount of an agent that produces some desired effect at a reasonable benefit/risk ratio. In certain embodiments, the term refers to that amount necessary or sufficient to treat Dry AMD or to cause return of previously lost visual acuity in a subject who suffers from Dray AMD. The effective amount may vary depending on such factors as the disease or condition being treated, the particular composition being administered, or the severity of the disease or condition. One of skill in the art may empirically determine the effective amount of a particular agent without necessitating undue experimentation.

This application discloses additional data, information and therapeutic uses for Risuteganib. Risuteganib is shown to cause a number of effects, including the following:
Deterrence of angiogenesis and possible regression of neovascularization by downregulating production of VEGF and other proangiogenic growth factors including ANG-2; Suppression of retinal angiogenesis in OIR, CNV and hVEGF mouse models; Inhibiting endothelial adhesion and migration on matrix-coated surfaces and suppression of endothelial cell proliferation
Reduction of vascular leakage by inhibiting the production of VEGF and inflammatory mediators;
Reduction of inflammation, at least in part by targeting multiple integrin subunits; Reducing expression of the Complement 3 Receptor (also known as Integrin αMβ2); Reduction of leucocyte adhesion; Reduction of trans-endothelial leucocyte migration; and Reductions of TNF-α pathway gene expression in human immune cells2; Lowering pro-inflammatory cytokine levels (e.g., in corneal tissue).
Neuroprotection/Neuroregeration/Restoration of lost or impaired nerve function by decreasing apoptosis, increasing cell survival (e.g., in a ROP Model); Reducing free radical oxygen production; Enhancing mitochondrial health; Stabilizing and deterring leakage from mitochondrial cell membranes; Improving retinal and/or optic nerve function; Improving vision; Improving vision or restoring previously lost visual acuity in subjects suffering from retinal and/or optic nerve degeneration or damage (e.g., due to dry macular degeneration, glaucoma, hereditary or familial retinal and/or macular disorders including but not limited to Leber congenital amaurosis, choroideremia, Stargardt's disease, Usher Syndrome and achromatopsia; Other hereditary dystrophies affecting the central retina; Retinal and/or optic nerve degeneration due to mutations in gene(s) responsible for changes of the choroid (e.g., choroideremia) or retinal pigment epithelium (RPE)(e.g., Best's disease)); Treating degeneration of photoreceptor outer segments (e.g., Stargardt's disease); Treating impaired color vision; Treating degeneration of bipolar and/or Mueller cells (e.g., x-linked retinoschisis); Increasing mitochondrial membrane potential; Improving mitochondrial bioenergetics; Reducing mitochondrial reactive oxygen species (ROS) in tissues under mechanical, oxidative, hypoxic, anoxic, chemical, chemo-toxic or other stress (e.g., in retinal tissue following H₂O₂ and hydroquinone exposure.

### Risuteganib Treatment of Dry AMD in Human Subjects

Eligible subjects who had been diagnosed with intermediate non-exudative AMD that required treatment were enrolled and randomized to either Group 1 or Group 2. Twenty-five subjects were assigned to Group 1 and fifteen (15) subjects were assigned to Group 2. Study treatments were administered to the subjects in Groups 1 and 2, as follows:
Each subject assigned to Group 1 received a first treatment consisting of a sham injection in the study eye on day 1 of the study and then crossed over to receive a second treatment consisting of an intravitreal injection into the study eye of 1.0 mg/50 μL risuteganib during week 16 of the study.
Each subject assigned to the Group 2 received a first treatment consisting of an intravitreal injection into the study eye of 1.0 mg/50 μL risuteganib (i.e., 1.0 mg in 50 μL of isotonic saline solution) on day 1 of the study and a second treatment consisting of an intravitreal injection into the study eye of 1.0 mg/50 μL of risuteganib during week 16 of the study.

The subjects in Groups 1 and 2 received the following treatments: Thus, subjects in Group 1 received an initial sham injection in the study eye followed by a single 1 mg dose of risuteganib in the study eye. The subjects in Group 2 received a total of two (2) doses of risuteganib (1 mg per dose) in the study eye.

Numerous study assessments were conducted at various time points throughout the study. Included among these study assessments were; refractive eye examinations, determinations of BCVA AND low-luminance BCVA, Lanthony D-15 color vision test, measurement of intraocular pressure (IOP), Indirect ophthalmoscopy/dilated fundus examinations and spectral-domain optical coherence tomography (SD-OCT). Also, blood and saliva samples were obtained from each subject for genetic analysis. The above-listed study assessments were performed at the time points indicated in Table 1, below:

TABLE 1
Schedule of Visits and Assessments

| | | Baseline/ | | | | | | | | |
|---|---|---|---|---|---|---|---|---|---|---|
| | Screening | Day 1 | Week 4 | Week 8 | Week 12 | Week 16 | Week 20 | Week 24 | Week 28 | Week 32 |
| | Visit | Visit | Visit | Visit | Visit | Visit | Visit | Visit | Visit | Visit |
| | Visit 1 | Visit 2 | Visit 3 | Visit 4 | Visit 5 | Visit 6 | Visit 7 | Visit 8 | Visit 9 | Visit 10 |
| | (−28 to −2 | (±1 | (±3 | (±3 | (±3 | (±3 | (±3 | (±3 | (±3 | (±3 |
| Visit | days) | day) | days) | days) | days) | days) | days) | days) | days) | days) |
| Refraction | X | X | X | X | X | X | X | X | X | X |
| and BCVA | | | | | | | | | | |
| Low- | X | | | | X | | | | | X |
| luminance | | | | | | | | | | |
| BCVA | | | | | | | | | | |
| Lanthony | X | | | | X | | | | | X |
| D-15 color | | | | | | | | | | |
| vision test | | | | | | | | | | |
| IOP | X | X | X | X | X | X | X | X | X | X |
| Indirect | X | X | X | X | X | X | X | X | X | X |
| ophthalmoscopy/ | | | | | | | | | | |
| dilated | | | | | | | | | | |
| fundus | | | | | | | | | | |
| exam | | | | | | | | | | |
| SD-OCT | X | | | | X | | | | | X |

| | |
|---|---|
| Blood or | X |
| saliva | |
| sample for | |
| genetic | |
| analysis | |
| [a] | |

### Primary Efficacy Outcomes:

For this study, a primary efficacy endpoint was deemed to be the percentage of population with an improvement in BCVA of at least 8 letters (1.5 lines) BCVA. Table 2, below, summarizes the proportion of Group 2 subjects who exhibited this primary efficacy outcome at Week 12 and the proportion of Group 1 subjects who exhibited this primary efficacy outcome at Week 28 of the study:

TABLE 2
Proportion of Subjects With Gain of 8 or More
BCVA Letters Read at Primary Endpoint Week

| | GROUP 1 | | GROUP 2 | |
|---|---|---|---|---|
| | Week 12 | | Week 28 | |
| | n = 14 | | n = 25 | |
| Gain of ≥8 letters read, n (%) | 1 | (7.1) | 12 | (48.0) |
| 95% exact CI | 0.18, 33.87 | | 27.80, 68.69 | |
| Baseline visit, letters read | | | | |
| N | 14 | | 25 | |
| Mean (SD) | 67.1 | (4.99) | 64.4 | (6.74) |
| 95% CI | 64.26, 70.02 | | 61.62, 67.18 | |
| Median | 69.5 | | 66.0 | |
| Min, Max | 57, 73 | | 45, 73 | |
| Primary endpoint week,^{a} letters read | | | | |
| N | 14 | | 25 | |
| Mean (SD) | 69.3 | (8.64) | 70.5 | (8.03) |
| 95% CI | 64.30, 74.28 | | 67.20, 73.84 | |
| Median | 71.0 | | 71.0 | |
| Min, Max | 51, 83 | | 57, 87 | |
| Change in letters read | | | | |
| N | 14 | | 25 | |
| Mean (SD) | 2.1 | (5.04) | 6.1 | (7.60) |

| | | |
|---|---|---|
| 95% CI | −0.76, 5.05 | 2.98, 9.26 |
| Median | 2.0 | 6.0 |
| Min, Max | −6, 10 | −6, 20 |

Abbreviations: CI, confidence interval; max, maximum; min, minimum; SD, standard deviation.
Primary endpoint week was Week 12 for the sham group and Week 28 for the risuteganib group.

It was determined that, at baseline, no anatomical measurements showed a significant difference between risuteganib nonresponder eyes and sham eyes.

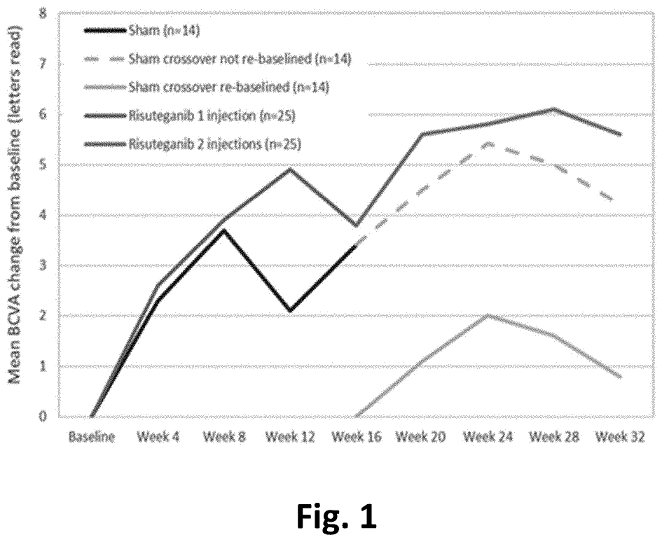

FIG. 1 is a graph showing mean change in BCVA visit in a study of human Subjects suffering from Dry AMD. The proportion of subjects with a gain of at least 8 BCVA letters read was 48% in Group 2 at Week 28 compared with 7.1% in Group 1 at Week 12. Although hypothesis testing was not planned, post hoc analysis using a 2-sided Fisher's exact test demonstrated that this was a statistically significant difference between groups (P=0.013).

Additional post hoc analysis was performed to assess whether the presence of foveal geographic atrophy (GA) in risuteganib-treated subjects affected the degree of BCVA improvement. The Group 2 subjects were divided into 2 subgroups: those with eyes with no foveal geographic atrophy (GA) in the central 6-mm subfield (the “No GA Subgroup”) and those with GA in the central 6-mm subfield (the “GA Subgroup”). The proportion of risuteganib-treated subjects with a gain of at least 8 BCVA letters read was higher in the No GA Subgroup when compared to the GA Subgroup (80% vs 40%).

### Secondary Efficacy Outcomes:

Secondary efficacy outcomes were deemed to be the following:
Mean Observed Changes in BCVA Between the Group 1 at Week 12 and Group 2 at Week 28;
Mean Observed Changes in BCVA Between Groups 1 and 2 at Week 12;
Maximum Observed Changes in BCVA Between Groups 1 and 2; and
Percentage of all subjects who exhibited an improvement in BCVA of at least 8 letters (1.5 lines) BCVA.

Table 3, below, summarizes mean BCVA change over time in the subset of subjects who met or exceeded the primary endpoint criteria:

TABLE 3
Mean BCVA Change Over Time in the Subset of Subjects With Gain
of 8 or More BCVA Letters Read at Primary Endpoint Week

| | GROUP 1 | | GROUP 1 | | GROUP 2 | |
|---|---|---|---|---|---|---|
| | Week 0 to Week 16 | | Week 16 to Week 32 | | Week 0 to Week 32 | |
| | n = 1 | | n = 2 | | n = 12 | |
| Baseline visit, letters read | | | | | | |
| N | 1 | | | | 12 | |
| Mean (SD) | 73.0 | (NA) | | | 62.9 | (7.27) |
| 95% CI | | | | | 58.30, 67.53 | |
| Median | 73.0 | | | | 65.0 | |
| Min, Max | 73, 73 | | | | 45, 71 | |
| Week 4, letters read | | | | | | |
| N | 1 | | | | 12 | |
| Mean (SD) | 72.0 | (NA) | | | 67.0 | (10.07) |
| 95% CI | | | | | 60.60, 73.40 | |
| Median | 72.0 | | | | 68.5 | |
| Min, Max | 72, 72 | | | | 44, 81 | |
| Week 4 change in letters read | | | | | | |
| N | 1 | | | | 12 | |
| Mean (SD) | −1.0 | (NA) | | | 4.1 | (7.15) |
| 95% CI | | | | | −0.46, 8.63 | |
| Median | −1.0 | | | | 3.0 | |
| Min, Max | −1, −1 | | | | −5, 22 | |
| Week 8, letters read | | | | | | |
| N | 1 | | | | 12 | |
| Mean (SD) | 85.0 | (NA) | | | 68.5 | (10.80) |
| 95% CI | | | | | 61.64, 75.36 | |
| Median | 85.0 | | | | 72.0 | |
| Min, Max | 85, 85 | | | | 50, 81 | |
| Week 8 change in letters read | | | | | | |
| N | 1 | | | | 12 | |
| Mean (SD) | 12.0 | (NA) | | | 5.6 | (6.92) |
| 95% CI | | | | | 1.19, 9.98 | |
| Median | 12.0 | | | | 5.5 | |
| Min, Max | 12, 12 | | | | −6, 18 | |
| Week 12, letters read | | | | | | |
| N | 1 | | | | 12 | |
| Mean (SD) | 83.0 | (NA) | | | 70.6 | (11.08) |
| 95% CI | | | | | 63.54, 77.62 | |
| Median | 83.0 | | | | 72.0 | |
| Min, Max | 83, 83 | | | | 47, 87 | |
| Week 12 change in letters read | | | | | | |
| N | 1 | | | | 12 | |
| Mean (SD) | 10.0 | (NA) | | | 7.7 | (6.61) |
| 95% CI | | | | | 3.47, 11.87 | |
| Median | 10.0 | | | | 5.5 | |
| Min, Max | 10, 10 | | | | −1, 21 | |
| Week 16, letters read | | | | | | |
| N | 1 | | 2 | | 12 | |
| Mean (SD) | 81.0 | (NA) | 70.0 | (7.07) | 69.2 | (9.45) |
| 95% CI | | | 6.47, 133.53 | | 63.16, 75.17 | |
| Median | 81.0 | | 70.0 | | 70.5 | |
| Min, Max | 81, 81 | | 65, 75 | | 52, 80 | |
| Week 16 change in letters read | | | | | | |
| N | 1 | | | | 12 | |
| Mean (SD) | 8.0 | (NA) | | | 6.3 | (6.43) |
| 95% CI | | | | | 2.17, 10.33 | |
| Median | 8.0 | | | | 8.0 | |
| Min, Max | 8, 8 | | | | −6, 15 | |
| Week 20, letters read | | | | | | |
| N | | | 2 | | 12 | |
| Mean (SD) | | | 70.0 | (7.07) | 74.2 | (8.03) |
| 95% CI | | | 6.47, 133.53 | | 69.06, 79.27 | |
| Median | | | 70.0 | | 75.0 | |
| Min, Max | | | 65, 75 | | 58, 90 | |
| Week 20 change in letters read | | | | | | |
| N | | | 2 | | 12 | |
| Mean (SD) | | | 0.0 | (0.00) | 11.3 | (4.56) |
| 95% CI | | | | | 8.36, 14.14 | |
| Median | | | 0.0 | | 10.0 | |
| Min, Max | | | 0, 0 | | 5, 20 | |
| Week 24, letters read | | | | | | |
| N | | | 2 | | 12 | |
| Mean (SD) | | | 78.0 | (2.83) | 74.3 | (7.88) |
| 95% CI | | | 52.59, 103.41 | | 69.33, 79.34 | |
| Median | | | 78.0 | | 75.5 | |
| Min, Max | | | 76, 80 | | 56, 85 | |
| Week 24 change in letters read | | | | | | |
| N | | | 2 | | 12 | |
| Mean (SD) | | | 8.0 | (4.24) | 11.4 | (4.34) |
| 95% CI | | | −30.12, 46.12 | | 8.66, 14.17 | |
| Median | | | 8.0 | | 10.0 | |
| Min, Max | | | 5, 11 | | 6, 21 | |
| Week 28, letters read | | | | | | |
| N | | | 2 | | 12 | |
| Mean (SD) | | | 79.5 | (7.78) | 75.7 | (7.66) |
| 95% CI | | | 9.62, 149.38 | | 70.80, 80.53 | |
| Median | | | 79.5 | | 75.5 | |
| Min, Max | | | 74, 85 | | 57, 87 | |
| Week 28 change in letters read | | | | | | |
| N | | | 2 | | 12 | |
| Mean (SD) | | | 9.5 | (0.71) | 12.8 | (4.20) |
| 95% CI | | | 3.15, 15.85 | | 10.08, 15.42 | |
| Median | | | 9.5 | | 12.0 | |
| Min, Max | | | 9, 10 | | 8, 20 | |
| Week 32, letters read | | | | | | |
| N | | | 2 | | 12 | |
| Mean (SD) | | | 76.5 | (4.95) | 72.4 | (8.78) |
| 95% CI | | | 32.03, 120.97 | | 66.84, 78.00 | |
| Median | | | 76.5 | | 74.0 | |
| Min, Max | | | 73, 80 | | 57, 85 | |
| Week 32 change in letters read | | | | | | |
| N | | | 2 | | 12 | |
| Mean (SD) | | | 6.5 | (2.12) | 9.5 | (5.00) |

| | | |
|---|---|---|
| 95% CI | −12.56, 25.56 | 6.32, 12.68 |
| Median | 6.5 | 10.5 |
| Min, Max | 5, 8 | −2, 15 |

Abbreviations: CI, confidence interval; max, maximum; min, minimum; NA; not applicable; SD, standard deviation.

Table 4, below, summarizes the change in BCVA over time at any week in the study:

TABLE 4
Mean BCVA Change Over Time in the Subset of Subjects
With Gain of 8 or More BCVA Letters Read at Any Week

| | GROUP 1 | | GROUP 1 | | GROUP 2 | |
|---|---|---|---|---|---|---|
| | Week 0 to Week 16 | | Week 16 to Week 32 | | Week 0 to Week 32 | |
| | n = 7 | | n = 3 | | n = 14 | |
| Baseline visit, letters read | | | | | | |
| N | 7 | | | | 14 | |
| Mean (SD) | 69.9 | (2.91) | | | 62.5 | (6.81) |
| 95% CI | 67.16, 72.55 | | | | 58.57, 66.43 | |
| Median | 70.0 | | | | 63.5 | |
| Min, Max | 64, 73 | | | | 45, 71 | |
| Week 4, letters read | | | | | | |
| N | 7 | | | | 14 | |
| Mean (SD) | 72.1 | (6.12) | | | 66.5 | (9.52) |
| 95% CI | 66.48, 77.80 | | | | 61.00, 72.00 | |
| Median | 72.0 | | | | 67.0 | |
| Min, Max | 63, 83 | | | | 44, 81 | |
| Week 4 change in letters read | | | | | | |
| N | 7 | | | | 14 | |
| Mean (SD) | 2.3 | (7.36) | | | 4.0 | (6.66) |
| 95% CI | −4.53, 9.10 | | | | 0.16, 7.84 | |
| Median | −1.0 | | | | 3.0 | |
| Min, Max | −7, 14 | | | | −5, 22 | |
| Week 8, letters read | | | | | | |
| N | 7 | | | | 14 | |
| Mean (SD) | 75.7 | (6.10) | | | 68.3 | (10.07) |
| Median | 75.0 | | | | 71.0 | |
| Min, Max | 70, 85 | | | | 50, 81 | |
| Week 8 change in letters read | | | | | | |
| N | 7 | | | | 14 | |
| Mean (SD) | 5.9 | (5.52) | | | 5.8 | (6.44) |
| 95% CI | 0.75, 10.96 | | | | 2.07, 9.50 | |
| Median | 6.0 | | | | 5.5 | |
| Min, Max | −2, 12 | | | | −6, 18 | |
| Week 12, letters read | | | | | | |
| N | 7 | | | | 14 | |
| Mean (SD) | 75.9 | (4.14) | | | 69.6 | (10.54) |
| 95% CI | 72.03, 79.69 | | | | 63.56, 75.73 | |
| Median | 76.0 | | | | 69.5 | |
| Min, Max | 70, 83 | | | | 47, 87 | |
| Week 12 change in letters read | | | | | | |
| N | 7 | | | | 14 | |
| Mean (SD) | 6.0 | (3.00) | | | 7.1 | (6.53) |
| 95% CI | 3.23, 8.77 | | | | 3.37, 10.91 | |
| Median | 6.0 | | | | 5.5 | |
| Min, Max | 0, 10 | | | | −1, 21 | |
| Week 16, letters read | | | | | | |
| N | 7 | | 3 | | 14 | |
| Mean (SD) | 76.1 | (4.41) | 69.7 | (5.03) | 68.9 | (8.73) |
| 95% CI | 72.06, 80.22 | | 57.16, 82.17 | | 63.89, 73.97 | |
| Median | 75.0 | | 69.0 | | 69.5 | |
| Min, Max | 69, 81 | | 65, 75 | | 52, 80 | |
| Week 16 change in letters read | | | | | | |
| N | 7 | | | | 14 | |
| Mean (SD) | 6.3 | (2.14) | | | 6.4 | (6.09) |
| 95% CI | 4.31, 8.26 | | | | 2.91, 9.94 | |
| Median | 6.0 | | | | 8.0 | |
| Min, Max | 3, 9 | | | | −6, 15 | |
| Week 20, letters read | | | | | | |
| N | | | 3 | | 14 | |
| Mean (SD) | | | 71.7 | (5.77) | 72.3 | (9.22) |
| 95% CI | | | 57.32, 86.01 | | 66.96, 77.61 | |
| Median | | | 75.0 | | 74.5 | |
| Min, Max | | | 65, 75 | | 54, 90 | |
| Week 20 change in letters read | | | | | | |
| N | | | 3 | | 14 | |
| Mean (SD) | | | 2.0 | (3.46) | 9.8 | (6.62) |
| 95% CI | | | −6.61, 10.61 | | 5.96, 13.61 | |
| Median | | | 0.0 | | 10.0 | |
| Min, Max | | | 0, 6 | | −8, 20 | |
| Week 24, letters read | | | | | | |
| N | | | 3 | | 14 | |
| Mean (SD) | | | 78.3 | (2.08) | 72.7 | (8.34) |
| 95% CI | | | 73.16, 83.50 | | 67.90, 77.53 | |
| Median | | | 79.0 | | 74.5 | |
| Min, Max | | | 76, 80 | | 56, 85 | |
| Week 24 change in letters read | | | | | | |
| N | | | 3 | | 14 | |
| Mean (SD) | | | 8.7 | (3.21) | 10.2 | (5.16) |
| 95% CI | | | 0.68, 16.65 | | 7.23, 13.19 | |
| Median | | | 10.0 | | 9.5 | |
| Min, Max | | | 5, 11 | | 0, 21 | |
| Week 28, letters read | | | | | | |
| N | | | 3 | | 14 | |
| Mean (SD) | | | 77.7 | (6.35) | 73.9 | (8.42) |
| 95% CI | | | 61.89, 93.44 | | 69.00, 78.72 | |
| Median | | | 74.0 | | 74.5 | |
| Min, Max | | | 74, 85 | | 57, 87 | |
| Week 28 change in letters read | | | | | | |
| N | | | 3 | | 14 | |
| Mean (SD) | | | 8.0 | (2.65) | 11.4 | (5.26) |
| 95% CI | | | 1.43, 14.57 | | 8.32, 14.39 | |
| Median | | | 9.0 | | 11.5 | |
| Min, Max | | | 5, 10 | | 2, 20 | |
| Week 32, letters read | | | | | | |
| N | | | 3 | | 14 | |
| Mean (SD) | | | 76.0 | (3.61) | 70.7 | (9.19) |
| 95% CI | | | 67.04, 84.96 | | 65.41, 76.02 | |
| Median | | | 75.0 | | 73.0 | |
| Min, Max | | | 73, 80 | | 57, 85 | |
| Week 32 change in letters read | | | | | | |
| N | | | 3 | | 14 | |
| Mean (SD) | | | 6.3 | (1.53) | 8.2 | (5.65) |

| | | |
|---|---|---|
| 95% CI | 2.54, 10.13 | 4.95, 11.47 |
| Median | 6.0 | 9.5 |
| Min, Max | 5, 8 | −2, 15 |

Abbreviations: CI, confidence interval; max, maximum; min, minimum; SD, standard deviation.

### Color Vision Test

The results of color vision testing of the study subjects are summarized in Table 5, below.

TABLE 5
Color Vision as Measured by Total Error Score Hue Style

| | |
|---|---|
| GROUP 1 | GROUP 2 |
| n = 14 | n = 25 |

Screening

| | | |
|---|---|---|
| N | 14 | 25 |

| | | | | |
|---|---|---|---|---|
| Mean (SD) | 50.52 | (31.192) | 43.27 | (28.678) |

| | | |
|---|---|---|
| 95% CI | 32.515, 68.534 | 31.429, 55.105 |
| Median | 47.59 | 44.67 |
| Min, Max | 4.7, 101.0 | 0.0, 99.3 |

Week 12

| | | |
|---|---|---|
| N | 13 | 23 |

| | | | | |
|---|---|---|---|---|
| Mean (SD) | 48.61 | (33.835) | 43.38 | (30.099) |

| | | |
|---|---|---|
| 95% CI | 28.168, 69.061 | 30.361, 56.393 |
| Median | 48.00 | 39.67 |
| Min, Max | 1.3, 121.7 | 1.3, 89.3 |

Week 12 change

| | | |
|---|---|---|
| N | 13 | 23 |

| | | | | |
|---|---|---|---|---|
| Mean (SD) | 1.97 | (17.919) | 2.41 | (17.964) |

| 95% CI | −8.855, 12.801 | −5.363, 10.174 |
|---|---|---|
| Median | 5.33 | 2.67 |

| | | |
|---|---|---|
| Min, Max | −28.2, 40.7 | −44.2, 35.3 |

Week 32

| | | |
|---|---|---|
| N | 14 | 24 |

| | | | | |
|---|---|---|---|---|
| Mean (SD) | 48.76 | (34.018) | 39.88 | (33.181) |

| | | |
|---|---|---|
| 95% CI | 29.121, 68.403 | 25.864, 53.887 |
| Median | 42.00 | 24.09 |
| Min, Max | 6.7, 107.5 | 0.0, 104.0 |

Week 32 change

| | | |
|---|---|---|
| N | 14 | 24 |

| | | | | |
|---|---|---|---|---|
| Mean (SD) | −1.76 | (22.474) | −4.36 | (20.808) |

| | | |
|---|---|---|
| 95% CI | −14.738, 11.214 | −13.147, 4.426 |
| Median | 1.34 | −3.17 |
| Min, Max | −67.7, 26.5 | −40.2, 42.7 |

Abbreviations: CI, confidence interval; max, maximum; min, minimum; SD, standard deviation.

As shown in Table 5 above, the mean total color vision error score in Group 1 subjects at screening (pre-treatment) was 50.52. At Week 12, the mean color vision score of Group 1 subjects had increased (worsening of color vision) by 1.97. Following crossover and administration of the single dose of risuteganib, the mean total color vision error score in Group 1 subjects decreased (improved) by 1.76 at Week 32.

As shown in Table 5 above, the mean total error score on the color vision test for Group 2 subjects was 43.27 at screening. This score increased in the Group 2 subjects (worsening of color vision) by 2.41 at Week 12 and then decreased (improvement in color vision) by 4.36 at Week 32.

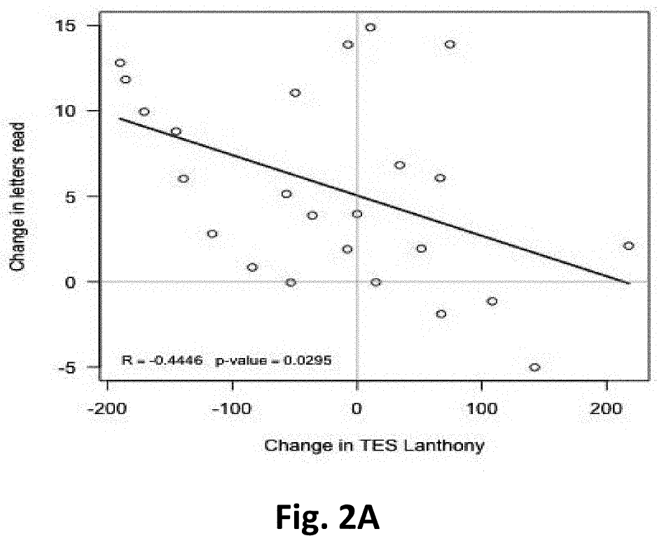

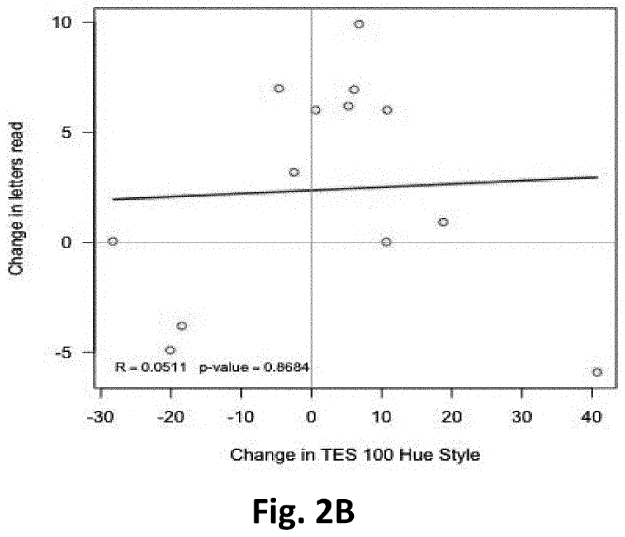

FIGS. 2A and 2B show analysis of scatter plots of change in total error score by change in BCVA letters read from baseline at Week 12. FIG. 2A shows a negative correlation for Group 2 subjects at 12 weeks following their initial risuteganib dose (decreased color vision scores correlate with increased BCVA) and FIG. 2B shows a slight positive correlation for Group 1 subjects at 12 weeks following their initial sham injection.

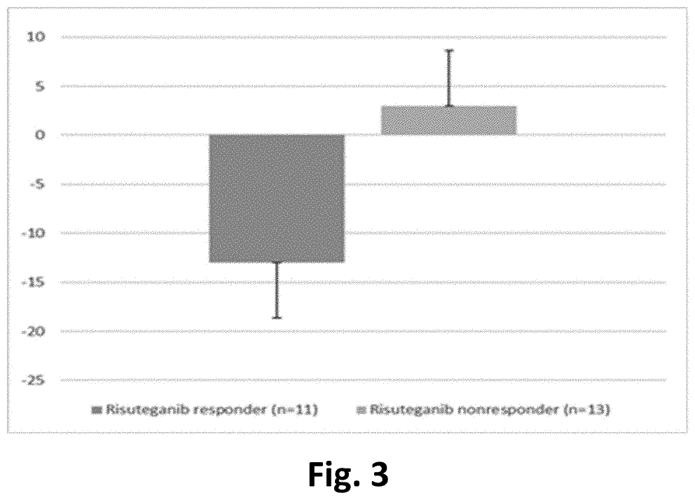

Examination of change in total error score by responder status (subjects with or without a letters BCVA gain) shows that risuteganib responders at Week 32 had a decrease (improvement) in color vision of 13.03 compared with an increase (worsening) of 2.98 for sham responders at Week 12n as seen in the bar graph of FIG. 3.

### Improvement in Perimetry Humphrey Visual Field Assessment

Table 6, below, shows mean deviation (MD) scores from the Humphrey visual field assessment, which compares subject performance to an age-matched normative database.

TABLE 6
Humphrey Visual Field as Measured by Mean Deviation

| | Sham or Crossover to | | | |
|---|---|---|---|---|
| | Risuteganib | | Risuteganib | |
| | n = 14 | | n = 25 | |
| Screening, dB | | | | |
| N | 12 | | 21 | |
| Mean (SD) | −4.074 | (4.6813) | −4.557 | (4.0715) |
| 95% CI | −7.0485, −1.0998 | | −6.4105, −2.7038 | |
| Median | −2.455 | | −3.330 | |
| Min, Max | −16.19, −0.44 | | −18.58, −0.48 | |
| Week 12, dB | | | | |
| N | 8 | | 21 | |
| Mean (SD) | −4.665 | (4.8504) | −5.502 | (6.6203) |
| 95% CI | −8.7201, −0.6099 | | −8.5154, −2.4884 | |
| Median | −2.870 | | −3.500 | |
| Min, Max | −14.45, 0.02 | | −25.00, 0.66 | |
| Week 12 change, dB | | | | |
| N | 7 | | 17 | |
| Mean (SD) | 0.561 | (0.9252) | 0.302 | (1.7590) |
| 95% CI | −0.2942, 1.4171 | | −0.6026, 1.2061 | |
| Median | 0.590 | | 0.100 | |
| Min, Max | −0.90, 1.74 | | −2.69, 3.21 | |
| Week 32, dB | | | | |
| N | 11 | | 21 | |
| Mean (SD) | −4.055 | (5.1026) | −5.211 | (5.5763) |
| 95% CI | −7.4834, −0.6275 | | −7.7493, −2.6727 | |
| Median | −2.260 | | −3.470 | |
| Min, Max | −16.19, 0.54 | | −25.33, −1.30 | |
| Week 32 change, dB | | | | |
| N | 10 | | 16 | |
| Mean (SD) | 0.158 | (0.7268) | 0.191 | (1.1383) |

| | | |
|---|---|---|
| 95% CI | −0.3619, 0.6779 | −0.4153, 0.7978 |
| Median | −0.070 | −0.040 |
| Min, Max | −0.58, 1.73 | −1.70, 1.94 |

Abbreviations: CI, confidence interval; dB, decibels; max, maximum; min, minimum; SD, standard deviation.
NOTE:
only measures of “acceptable” quality were included.

In the sham group, the mean MD score was −4.074 dB at screening. This score increased (improved) by 0.561 dB at Week 12; after crossover to 1 risuteganib injection, this score increased by 0.158 dB at Week 32. In the risuteganib group, the mean MD score was −4.557 dB at screening. This score increased by 0.302 dB at Week 12 and by 0.191 dB at Week 32.

Table 7, below, shows pattern standard deviation (PSD) scores from the Humphrey visual field assessment, which can identify focal defects.

TABLE 7
Humphrey Visual Field as Measured by Pattern Standard Deviation

Screening, dB

| | | |
|---|---|---|
| N | 12 | 21 |

| | | | | |
|---|---|---|---|---|
| Mean (SD) | 2.401 | (1.5819) | 3.352 | (3.2841) |

| | | |
|---|---|---|
| 95% CI | 1.3957, 3.4060 | 1.8570, 4.8468 |
| Median | 2.150 | 1.660 |
| Min, Max | 1.18, 7.15 | 1.13, 13.27 |

Week 12, dB

| | | |
|---|---|---|
| N | 8 | 21 |

| | | | | |
|---|---|---|---|---|
| Mean (SD) | 2.914 | (2.7491) | 3.350 | (3.5796) |

| | | |
|---|---|---|
| 95% CI | 0.6154, 5.2121 | 1.7201, 4.9789 |
| Median | 2.170 | 1.630 |
| Min, Max | 1.17, 9.45 | 1.10, 13.18 |

Week 12 change, dB

| | | |
|---|---|---|
| N | 7 | 17 |

| | | | | |
|---|---|---|---|---|
| Mean (SD) | 0.447 | (0.8439) | −0.340 | (0.8416) |

| | | |
|---|---|---|
| 95% CI | −0.3333, 1.2276 | −0.7727, 0.0927 |
| Median | 0.290 | −0.090 |
| Min, Max | −0.15, 2.30 | −2.69, 0.43 |

Week 32, dB

| | | |
|---|---|---|
| N | 11 | 21 |

| | | | | |
|---|---|---|---|---|
| Mean (SD) | 2.790 | (1.7152) | 3.113 | (2.7424) |

| | | |
|---|---|---|
| 95% CI | 1.6377, 3.9423 | 1.8650, 4.3616 |
| Median | 2.410 | 2.050 |
| Min, Max | 1.12, 7.15 | 1.17, 10.42 |

Week 32 change, dB

| | | |
|---|---|---|
| N | 10 | 16 |

| | | | | |
|---|---|---|---|---|
| Mean (SD) | 0.469 | (0.5951) | 0.115 | (0.9026) |

| | | |
|---|---|---|
| 95% CI | 0.0433, 0.8947 | −0.3660, 0.5960 |
| Median | 0.360 | 0.045 |
| Min, Max | −0.37, 1.72 | −1.94, 1.38 |

Abbreviations: CI, confidence interval; dB, decibels; max, maximum; min, minimum; SD, standard deviation.
NOTE:
only measures of “acceptable” quality were included.

In Group 1 subjects, the mean PSD score was 2.401 dB at screening (pre-treatment). This score increased in Group 1 subjects by 0.447 dB at Week 12. After crossover and administration of the single risuteganib injection, this score increased in the Group 1 subjects by 0.469 dB at Week 32.

In the Group 2 subjects, the mean PSD score was 3.352 dB at screening (pre-treatment). This score decreased by 0.340 dB at Week 12 and increased by 0.115 dB at Week 32.

### Retinal Sensitivity

Table 8, below, shows mean retinal sensitivity as measured by microperimetry.

TABLE 8
Microperimetry as Measured by Mean Sensitivity

| | |
|---|---|
| GROUP 1 | GROUP 2 |
| n = 14 | n = 25 |

Screening

| | | |
|---|---|---|
| N | 9 | 13 |

| | | | | |
|---|---|---|---|---|
| Mean (SD) | 12.43 | (5.199) | 8.52 | (5.006) |

| | | |
|---|---|---|
| 95% CI | 8.437, 16.430 | 5.490, 11.540 |
| Median | 15.10 | 10.40 |
| Min, Max | 3.1, 17.8 | 0.4, 16.7 |

eek 12

| | | |
|---|---|---|
| N | 7 | 14 |

| | | | | |
|---|---|---|---|---|
| Mean (SD) | 9.56 | (5.459) | 7.52 | (4.969) |

| | | |
|---|---|---|
| 95% CI | 4.509, 14.605 | 4.652, 10.390 |
| Median | 11.70 | 7.50 |
| Min, Max | 1.7, 16.0 | 0.0, 16.2 |

Week 12 change

| | | |
|---|---|---|
| N | 7 | 11 |

| | | | | |
|---|---|---|---|---|
| Mean (SD) | −1.49 | (3.975) | −0.85 | (2.711) |

| | | |
|---|---|---|
| 95% CI | −5.162, 2.190 | −2.676, 0.967 |
| Median | −0.60 | −1.50 |
| Min, Max | −6.4, 4.9 | −5.1, 3.9 |

Week 32

| | | |
|---|---|---|
| N | 8 | 12 |

| | | | | |
|---|---|---|---|---|
| Mean (SD) | 11.44 | (6.655) | 8.25 | (4.601) |

| | | |
|---|---|---|
| 95% CI | 5.873, 17.002 | 5.327, 11.173 |
| Median | 13.70 | 8.50 |
| Min, Max | 0.0, 17.3 | 0.0, 15.4 |

Week 32 change

| | | |
|---|---|---|
| N | 8 | 9 |

| | | | | |
|---|---|---|---|---|
| Mean (SD) | −2.16 | (5.527) | -0.53 | (4.373) |

| | | |
|---|---|---|
| 95% CI | −6.783, 2.458 | −3.895, 2.828 |
| Median | −0.20 | −0.40 |
| Min, Max | −12.9, 3.2 | −7.8, 4.2 |

Abbreviations: CI, confidence interval; max, maximum; min, minimum; SD, standard deviation.

As seen in Table 8, above, mean retinal sensitivity in Group 1 subjects was 12.43 dB at screening (pre-treatment). This score decreased in the Group 1 subjects (worsened) by 1.49 dB at Week 12. Following crossover and administration of the single risuteganib injection to the Group 1 subjects, the mean retinal sensitivity score in those subjects decreased by 2.16 dB at Week 32.

In Group 2 subjects, mean retinal sensitivity was 8.52 dB at screening (pre-treatment). This score decreased by 0.85 dB in Group 2 subjects at Week 12 and further decreased by 0.53 dB at Week 32.

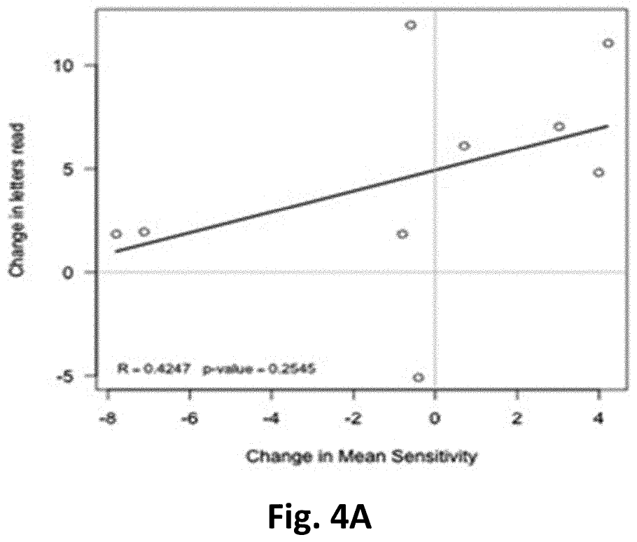

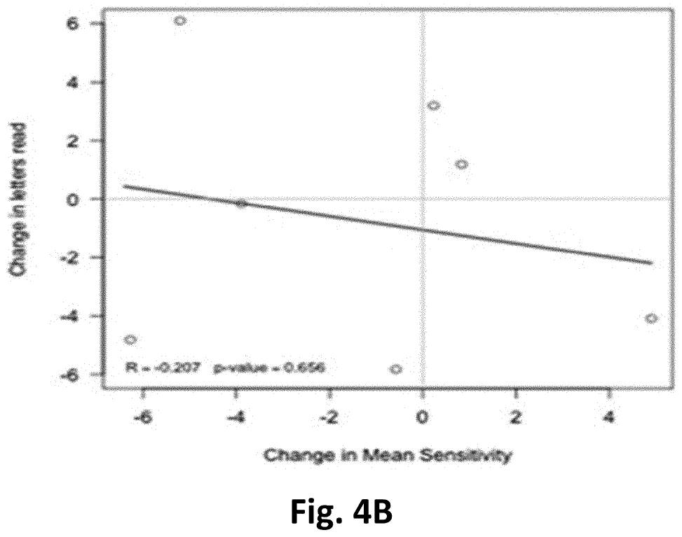

FIGS. 4A and 4B show scatter plots of change in mean sensitivity by change in BCVA letters read from baseline at Week 12. FIG. 4A shows a positive correlation for Group 2 subjects following their initial dose of risuteganib (increased mean sensitivity correlates with increased BCVA) and FIG. 4B shows a slight negative correlation for Group 1 subjects following their initial sham injection.

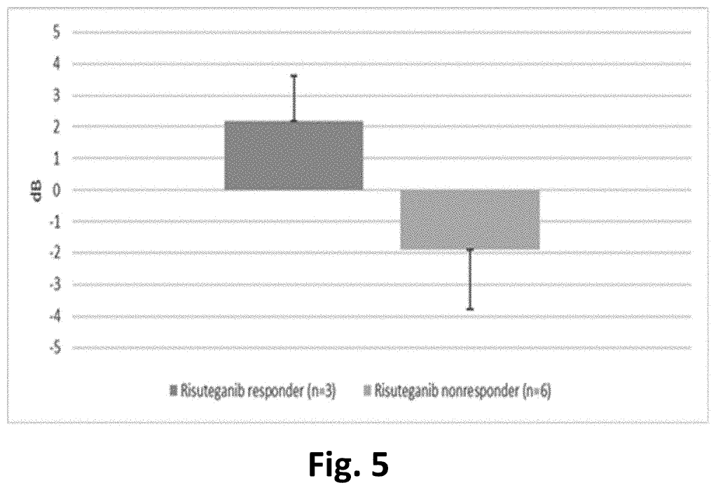

Examination of change in mean sensitivity by responder status showed that risuteganib responders at Week 32 had an increase (improvement) of 2.2 dB compared with a decrease (worsening) of 1.9 dB for sham responders at Week 12, as seen in the bar graph of FIG. 5.

Table 9, below, summarizes number of loci with reduced retinal sensitivity summed across assessments using a 20-dB threshold, an 11-dB threshold, and by measuring absolute scotoma.

TABLE 9
Microperimetry as Measured by Number of Loci Summed

| | |
|---|---|
| GROUP 1 | GROUP 2 |
| n = 14 | n = 25 |

Screening

| | | |
|---|---|---|
| N | 9 | 15 |

| | | | | |
|---|---|---|---|---|
| Mean (SD) | 65.4 | (23.38) | 81.4 | (24.23) |

| | | |
|---|---|---|
| 95% CI | 47.47, 83.41 | 67.98, 94.82 |
| Median | 56.0 | 74.0 |
| Min, Max | 46, 111 | 48, 123 |

Week 12

| | | |
|---|---|---|
| N | 7 | 14 |

| | | | | |
|---|---|---|---|---|
| Mean (SD) | 76.0 | (26.98) | 84.9 | (24.66) |

| | | |
|---|---|---|
| 95% CI | 51.05, 100.95 | 70.69, 99.17 |
| Median | 63.0 | 86.5 |
| Min, Max | 48, 122 | 47, 135 |

Week 12 change

| | | |
|---|---|---|
| N | 7 | 13 |

| | | | | |
|---|---|---|---|---|
| Mean (SD) | 5.1 | (15.42) | 6.1 | (25.04) |

| | | |
|---|---|---|
| 95% CI | −9.12, 19.40 | −9.06, 21.21 |
| Median | 11.0 | 4.0 |
| Min, Max | −26, 19 | −29, 69 |

Week 32

| | | |
|---|---|---|
| N | 8 | 12 |

| | | | | |
|---|---|---|---|---|
| Mean (SD) | 67.6 | (33.30) | 80.7 | (23.78) |

| | | |
|---|---|---|
| 95% CI | 39.78, 95.47 | 65.56, 95.78 |
| Median | 60.0 | 79.5 |
| Min, Max | 28, 127 | 53, 135 |

Week 32 change

| | | |
|---|---|---|
| N | 8 | 11 |

| | | | | |
|---|---|---|---|---|
| Mean (SD) | 7.9 | (27.46) | 1.0 | (20.89) |

| | | |
|---|---|---|
| 95% CI | −15.08, 30.83 | −13.03, 15.03 |
| Median | −1.0 | −3.0 |
| Min, Max | −22, 58 | −27, 36 |

Abbreviations: CI, confidence interval; max, maximum; mm, minimum; SD, standard deviation.

In the sham group, the mean number of summed loci with reduced sensitivity was 65.4 at screening. This score increased (worsened) by 5.1 at Week 12; after crossover to 1 risuteganib injection, this score increased by 7.9 at Week 32. In the risuteganib group, the mean number of summed loci with reduced sensitivity was 81.4 at screening. This score increased by 6.1 at Week 12 and by 1.0 at Week 32.

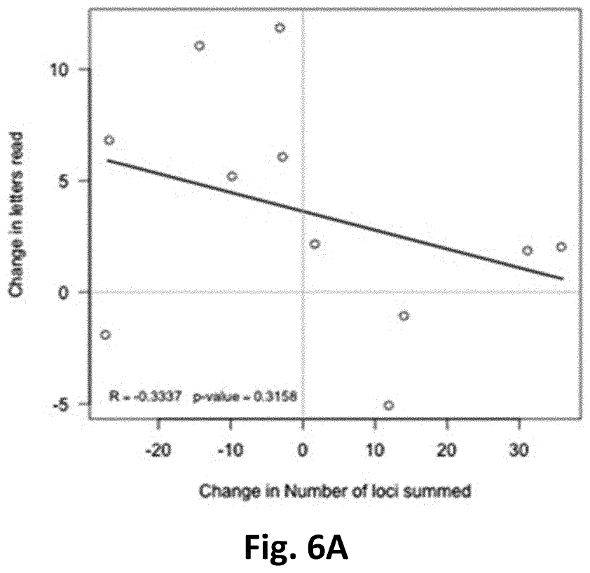

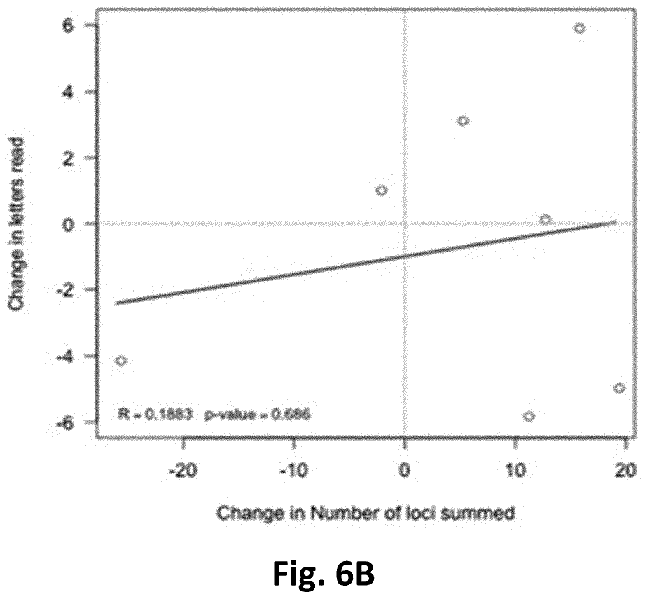

FIGS. 6A and 6B show scatter plots of change in number of loci with reduced retinal sensitivity by change in BCVA letters read from baseline at Week 12. FIG. 6A shows a negative correlation for Group 2 subjects following their initial risuteganib injection (decreased number of summed loci with reduced sensitivity correlates with increased BCVA) and FIG. 6B shows a slight positive correlation for Group 1 subjects following their initial sham injection. Error! Reference source not found.

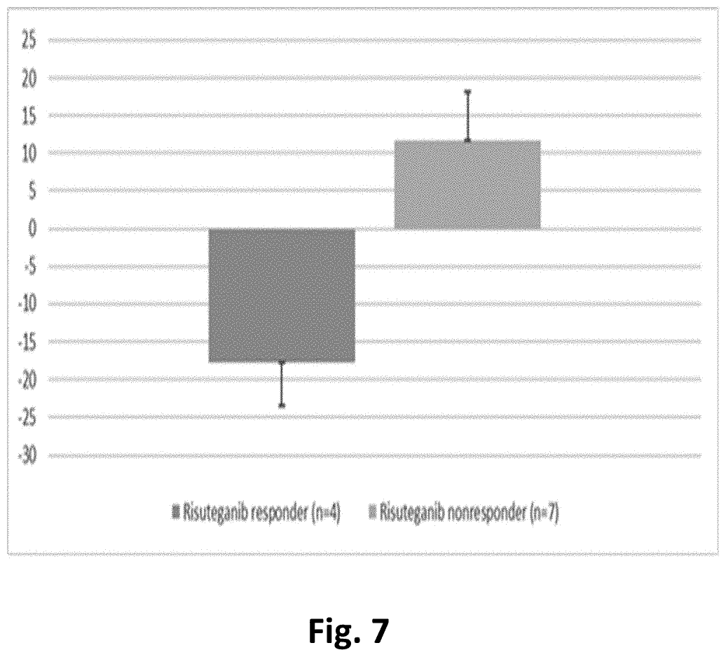

Examination of change in number of summed loci with reduced retinal sensitivity by responder status showed that risuteganib responders had a decrease (improvement) of 17.75 at Week 32 compared with an increase (worsening) of 11.71 at Week 12 for sham responders, as seen in the bar graph of FIG. 7. (P=0.014).

### Low-Luminance Visual Acuity

Table 10, below, summarizes low-luminescence visual acuity in the study subjects.

TABLE 10
Improvement in Low-Luminance Visual Acuity by Visit

| | GROUP 1 | | GROUP 2 | |
|---|---|---|---|---|
| | n = 14 | | n = 25 | |
| Screening, letters read | | | | |
| N | 14 | | 25 | |
| Mean (SD) | 48.1 | (7.40) | 47.4 | (12.26) |
| 95% CI | 43.87, 52.41 | | 42.30, 52.42 | |
| Median | 50.5 | | 50.0 | |
| Min, Max | 35, 56 | | 6, 68 | |
| Week 12, letters read | | | | |
| N | 13 | | 25 | |
| Mean (SD) | 48.8 | (9.91) | 46.4 | (12.51) |
| 95% CI | 42.86, 54.83 | | 41.19, 51.53 | |
| Median | 53.0 | | 48.0 | |
| Min, Max | 30, 63 | | 7, 71 | |
| Week 12 change in letters read | | | | |
| N | 13 | | 25 | |
| Mean (SD) | 0.9 | (8.68) | −1.0 | (6.95) |
| 95% CI | −4.32, 6.17 | | −3.87, 1.87 | |
| Median | 0.0 | | −1.0 | |
| Min, Max | −10, 18 | | −19, 17 | |
| Week 32, letters read | | | | |
| N | 14 | | 25 | |
| Mean (SD) | 50.7 | (17.58) | 49.4 | (12.50) |
| 95% CI | 40.57, 60.86 | | 44.24, 54.56 | |
| Median | 57.0 | | 51.0 | |
| Min, Max | 16, 75 | | 8, 69 | |
| Week 32 change in letters read | | | | |
| N | 14 | | 25 | |
| Mean (SD) | 2.6 | (16.59) | 2.0 | (7.95) |

| | | |
|---|---|---|
| 95% CI | −7.01, 12.15 | −1.24, 5.32 |
| Median | 3.0 | 0.0 |
| Min, Max | −27, 40 | −7, 24 |

Abbreviations: CI, confidence interval; max, maximum; min, minimum; SD, standard deviation.

As shown in Table 10 above, the mean low-luminance visual acuity in Group 1 subjects was 48.1 letters read at screening (pre-treatment). This score increased (improved) in the Group 1 subjects by 0.9 letters at Week 12. Following crossover and administration of the single risuteganib injection to the Group 1 subjects, this score increased by an additional 2.6 letters at Week 32.

Also, as shown in Table 10 above, the mean low-luminance visual acuity in Group 2 subjects was 47.4 letters read at screening. This score decreased (worsened) in Group 2 subjects by 1.0 letters at Week 12 and, thereafter, increased by 2.0 letters at Week 32.

### Retinal Examinations by Optical Coherence Tomography (OCT)

The OCT scans were analyzed by two (2) unrelated experts.

### OCT Analysis 1:

The mean thickness and mean volume of retinal subfields and layer segments were analyzed at screening (pre-treatment) and at Week 12 for Group 1 subjects and at Week 32 for Group 2 subjects. The results of this analysis are summarized in Table 11, below.

TABLE 11
Quantitative Anatomical Measurements at Baseline for
Risuteganib Nonresponder Eyes Versus Responder Eyes

| | Risuteganib | Risuteganib | |
|---|---|---|---|
| Measurement | Nonresponder | Responder | T-test |
| Layer, Sector | n = 12 | n = 10 | P-value |
| Mean thickness, μm | | | |
| Inner retina, foveal center | 27.833 | 42.500 | 0.305 |
| Inner retina, central subfield | 89.000 | 99.400 | 0.323 |
| Outer retina, foveal center | 124.417 | 143.200 | 0.210 |
| Outer retina, central subfield | 113.917 | 139.600 | 0.001 |
| Photoreceptor, foveal center | 46.833 | 48.500 | 0.784 |
| Photoreceptor, central subfield | 45.083 | 49.300 | 0.015 |
| RPEDC, foveal center | 47.667 | 58.900 | 0.540 |
| RPEDC, central subfield | 46.500 | 54.800 | 0.611 |
| Total volume, mm³ | | | |
| Inner retina, central subfield | 0.070 | 0.078 | 0.319 |
| Outer retina, central subfield | 0.090 | 0.110 | 0.001 |
| Photoreceptor, central subfield | 0.035 | 0.039 | 0.011 |
| RPEDC, central subfield | 0.037 | 0.043 | 0.600 |
| EZ defect area, mm² | 0.308 | 0.111 | 0.012 |

Abbreviations: EZ, ellipsoid zone; RPEDC, retinal pigment epithelium-drusen complex.

At baseline, those eyes that responded to risuteganib had significantly greater mean thickness in the central subfield of the outer retina compared with eyes that did not respond to risuteganib (139.600 vs 113.917 μm; P=0.001); responder eyes also had significantly greater mean thickness at baseline in the central subfield of the photoreceptor layer compared with nonresponder eyes (49.300 vs 45.083 μm; P=0.015; Table 11). The same anatomical locations also had significantly greater volume at baseline in the responder eyes compared with nonresponder eyes (central subfield of the outer retina, 0.110 vs 0.090 mm³; P=0.001 and central subfield of the photoreceptor layer, 0.039 vs 0.035 mm³; P=0.011). In addition, the EZ defect area of responder eyes was significantly smaller at baseline than that of nonresponders (0.111 vs 0.308 mm²; P=0.012). No other anatomical measurements showed a significant difference between risuteganib responder and nonresponder eyes at baseline.

In addition to the quantitative analysis of OCT images, a qualitative assessment of the OCT images at baseline (pre-treatment) was performed to identify GA anywhere in the retina, in the fovea (1-mm central subfield), and in the foveal center.

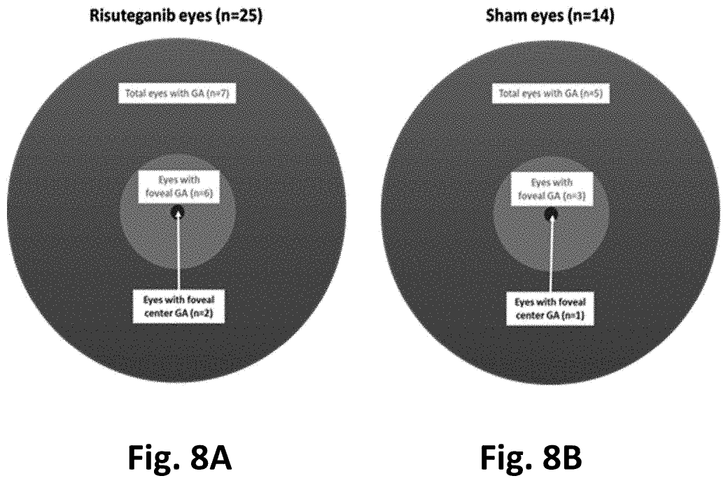

At baseline (pre-treatment), 7 of 25 (28%) of the eyes in Group 2 subjects had GA, 6 (24%) of which affected the fovea, and 2 (8%) of which involved the foveal center, as indicated on FIG. 8A. In addition, at baseline (pre-treatment), 5 of 14 (36%) Group 1 subject eyes had GA, 3 (26%) of which involved the fovea, and 1 (7%) of which affected the foveal center, as indicated on FIG. 8B. The relationship between functional visual acuity outcomes and the presence or absence of baseline GA is explored in the following Tables 12 and 13, respectively:

TABLE 12
Visual Acuity Functional Outcome in Study Eyes With Geographic Atrophy at Baseline

| Treatment | ≥8 Letter | | ≥10 Letter | | ≥15 Letter | |
|---|---|---|---|---|---|---|
| Location of | Improvement in | | Improvement in | | Improvement in | |
| Geographic | Visual Acuity | | Visual Acuity | | Visual Acuity | |
| Atrophy | n (%) | | n (%) | | n (%) | |
| Risuteganib | | | | | | |
| Geographic atrophy in retina (n = 7) | 2 | (29) | 2 | (29) | 1 | (14) |
| Geographic atrophy in fovea (n = 6) | 1 | (17) | 1 | (17) | 0 | (0) |
| Geographic atrophy in foveal center | 1 | (50) | 1 | (50) | 0 | (0) |
| (n = 2) | | | | | | |
| Sham | | | | | | |
| Geographic atrophy in retina (n = 5) | 0 | (0) | 0 | (0) | 0 | (0) |
| Geographic atrophy in fovea (n = 3) | 0 | (0) | 0 | (0) | 0 | (0) |
| Geographic atrophy in foveal center | 0 | (0) | 0 | (0) | 0 | (0) |
| (n = 1) | | | | | | |

TABLE 13
Visual Acuity Functional Outcome in Study Eyes Without Geographic Atrophy at Baseline

| Treatment | ≥8 Letter | | ≥10 Letter | | ≥15 Letter | |
|---|---|---|---|---|---|---|
| Location of | Improvement in | | Improvement in | | Improvement in | |
| Absent Geographic | Visual Acuity | | Visual Acuity | | Visual Acuity | |
| Atrophy | n (%) | | n (%) | | n (%) | |
| Risuteganib | | | | | | |
| No geographic atrophy in retina (n = 18) | 10 | (56) | 6 | (44) | 4 | (22) |
| No geographic atrophy in fovea (n = 19) | 11 | (58) | 7 | (37) | 5 | (26) |
| No geographic atrophy in foveal center | 11 | (48) | 7 | (30) | 5 | (22) |
| (n = 23) | | | | | | |
| Sham | | | | | | |
| No geographic atrophy in retina (n = 9) | 1 | (11) | 1 | (11) | 0 | (0) |
| No geographic atrophy in fovea (n = 11) | 1 | (9) | 1 | (9) | 0 | (0) |
| No geographic atrophy in foveal center | 1 | (8) | 1 | (8) | 0 | (0) |
| (n = 13) | | | | | | |

Since only one sham-treated eye had at least an 8-letter improvement in visual acuity, it is impossible to use the sham group to determine the effect of presence or absence of GA on functional outcomes. Therefore, the discussion below is focused on the risuteganib group.

Risuteganib-treated eyes without any GA at baseline (n=18) had a 56% responder rate when using an 8-letter improvement threshold compared with a 29% responder rate among risuteganib-treated eyes with any GA at baseline (n=7). The same pattern is maintained when using a 10-letter improvement (44% vs 29%, respectively) or a 15-letter improvement (22% vs 14%, respectively) as the visual acuity threshold.

Risuteganib-treated eyes without GA in the fovea at baseline (n=19) had a 58% responder rate (≥8-letter improvement threshold) compared with a 17% responder rate among risuteganib eyes with GA in the fovea at baseline (n=6). The same pattern is maintained when using a 10-letter improvement (37% vs 17%, respectively) or a 15-letter improvement (26% vs 0%, respectively) as the visual acuity threshold.

Risuteganib-treated eyes without GA in the foveal center at baseline (n=23) had a 48% responder rate (≥8-letter improvement threshold) compared with a 50% responder rate among risuteganib eyes with GA in the foveal center at baseline (n=2). However, because only 2 eyes had GA in the foveal center, the 50% responder rate in these eyes is not informative, and no conclusions can be drawn regarding the importance of GA under these circumstances.

Overall, these results suggest that absence of GA anywhere in the retina or at least in the central 1 mm (the area of the retina responsible for BCVA) increases the likelihood of response to risuteganib.

Quantitative analysis of the OCT images was also performed to measure changes in anatomical measurements over time. This analysis is summarized in Table 14 below.

TABLE 14
Quantitative Anatomical Measurements Change From Baseline at Week
32 for Risuteganib Nonresponder Eyes Versus Responder Eyes

| | Risuteganib | Risuteganib | | |
|---|---|---|---|---|
| Measurement | Nonresponder | Responder | | T-test |
| Layer, Sector | n = 12 | n = 10 | Difference | P-value |
| Mean change in mean thickness, μm | | | | |
| Inner retina, foveal center | 9.917 | 8.400 | −1.517 | 0.904 |
| Inner retina, central subfield | −2.250 | 5.200 | 7.450 | 0.042 |
| Outer retina, foveal center | −17.833 | −8.000 | 9.833 | 0.291 |
| Outer retina, central subfield | −5.417 | −2.400 | 3.017 | 0.261 |
| Photoreceptor, foveal center | 3.833 | 3.100 | −0.733 | 0.869 |
| Photoreceptor, central subfield | −1.333 | −1.000 | 0.333 | 0.849 |
| RPEDC, foveal center | −2.250 | −8.100 | −5.850 | 0.425 |
| RPEDC, central subfield | 1.083 | −9.800 | −10.883 | 0.307 |
| Mean change in total volume, mm³ | | | | |
| Inner retina, central subfield | −0.002 | 0.004 | 0.006 | 0.033 |
| Outer retina, central subfield | −0.004 | −0.002 | 0.003 | 0.223 |
| Photoreceptor, central subfield | −0.001 | −0.001 | 0.000 | 0.934 |
| RPEDC, central subfield | 0.001 | −0.008 | −0.009 | 0.297 |
| EZ defect area, mm² | 0.014 | 0.020 | 0.006 | 0.834 |

Abbreviations: EZ, ellipsoid zone; RPEDC, retinal pigment epithelium-drusen complex.

From baseline to Week 32, the central subfield of the inner retina in the risuteganib responder eyes had significantly larger increases in thickness (difference of 7.450 μm; P=0.042) and in volume (difference of 0.006 mm³; P=0.033) from baseline compared with risuteganib nonresponder eyes No other anatomical measurements showed a significant difference between responder and nonresponder eyes over time.

Significant differences in mean change from baseline to Week 32 in mean thickness for risuteganib eyes were observed compared with the mean change from baseline to Week 12 for sham eyes in the foveal center of the inner retina (difference of 15.404 μm; P=0.011), in the foveal center and central subfield of the outer retina (difference of −14.794 μm; P=0.007 and difference of −3.812 μm; P=0.042, respectively), and in the central subfield of the photoreceptor layer (difference of −2.545 μm; P=0.007). This is summarized in Table 15, below:

TABLE 15
Quantitative Anatomical Measurements Change From Baseline at Week 32 for
Risuteganib Arm Versus Change From Baseline at Week 12 for Sham Arm

| Measurement | Risuteganib | Sham | | T-test |
|---|---|---|---|---|
| Layer, Sector | n = 22 | n = 12 | Difference | P-value |
| Mean change in mean thickness, μm | | | | |
| Inner retina, foveal center | 6.696 | −8.708 | 15.404 | 0.011 |
| Inner retina, central subfield | 1.565 | 0.875 | 0.690 | 0.761 |
| Inner retina, nasal subfield | −0.783 | 0.167 | −0.949 | 0.609 |
| Inner retina, superior subfield | −3.022 | 1.167 | −4.188 | 0.059 |
| Inner retina, temporal subfield | 0.217 | 0.125 | 0.092 | 0.953 |
| Inner retina, inferior subfield | −0.065 | 1.542 | −1.607 | 0.393 |
| Outer retina, foveal center | −9.543 | 5.250 | −14.794 | 0.007 |
| Outer retina, central subfield | −3.978 | −0.167 | −3.812 | 0.042 |
| Outer retina, nasal subfield | −1.196 | −2.583 | 1.388 | 0.470 |
| Outer retina, superior subfield | −0.065 | −0.833 | 0.768 | 0.723 |
| Outer retina, temporal subfield | −2.283 | −1.125 | −1.158 | 0.484 |
| Outer retina, inferior subfield | −2.457 | −2.917 | 0.460 | 0.788 |
| Photoreceptor, foveal center | 1.717 | 0.375 | 1.342 | 0.608 |
| Photoreceptor, central subfield | −1.087 | 1.458 | −2.545 | 0.007 |
| Photoreceptor, nasal subfield | −0.087 | 0.292 | −0.379 | 0.407 |
| Photoreceptor, superior subfield | 0.130 | 0.292 | −0.161 | 0.716 |
| Photoreceptor, temporal subfield | −0.152 | 0.542 | −0.694 | 0.109 |
| Photoreceptor, inferior subfield | −0.239 | 0.542 | −0.781 | 0.123 |
| RPEDC, foveal center | −1.652 | −0.250 | −1.402 | 0.736 |
| RPEDC, central subfield | −1.500 | −1.333 | −0.167 | 0.969 |
| RPEDC, nasal subfield | 0.739 | 1.250 | −0.511 | 0.649 |
| RPEDC, superior subfield | 1.739 | −1.000 | 2.739 | 0.093 |
| RPEDC, temporal subfield | 0.870 | −1.667 | 2.536 | 0.099 |
| RPEDC, inferior subfield | 0.457 | −1.167 | 1.623 | 0.431 |
| Mean change in total volume, mm³ | | | | |
| Inner retina, central subfield | 0.001 | 0.001 | 0.001 | 0.740 |
| Inner retina, nasal subfield | −0.001 | 0.000 | −0.002 | 0.594 |
| Inner retina, superior subfield | −0.005 | 0.002 | −0.007 | 0.054 |
| Inner retina, temporal subfield | 0.000 | 0.000 | 0.000 | 0.914 |
| Inner retina, inferior subfield | 0.000 | 0.002 | −0.002 | 0.449 |
| Outer retina, central subfield | −0.003 | 0.000 | −0.003 | 0.035 |
| Outer retina, nasal subfield | −0.002 | −0.004 | 0.002 | 0.469 |
| Outer retina, superior subfield | −0.000 | −0.001 | 0.001 | 0.830 |
| Outer retina, temporal subfield | −0.004 | −0.002 | −0.002 | 0.508 |
| Outer retina, inferior subfield | −0.004 | −0.004 | 0.001 | 0.839 |
| Photoreceptor, central subfield | −0.001 | 0.001 | −0.002 | 0.009 |
| Photoreceptor, nasal subfield | −0.000 | 0.000 | −0.001 | 0.458 |
| Photoreceptor, superior subfield | 0.000 | 0.000 | −0.000 | 0.562 |
| Photoreceptor, temporal subfield | −0.000 | 0.001 | −0.001 | 0.128 |
| Photoreceptor, inferior subfield | −0.001 | 0.001 | −0.002 | 0.041 |
| RPEDC, central subfield | −0.001 | −0.001 | −0.000 | 0.989 |
| RPEDC, nasal subfield | 0.001 | 0.002 | −0.001 | 0.519 |
| RPEDC, superior subfield | 0.003 | −0.002 | 0.005 | 0.073 |
| RPEDC, temporal subfield | 0.001 | −0.003 | 0.004 | 0.084 |
| RPEDC, inferior subfield | 0.001 | −0.002 | 0.002 | 0.481 |
| EZ defect area, mm² | 0.015 | −0.010 | 0.025 | 0.210 |

Abbreviations: EZ, ellipsoid zone; RPEDC, retinal pigment epithelium-drusen complex.

As shown in the above Table 15, significant differences in mean change in total volume from baseline to Week 32 for risuteganib eyes were also observed compared with the mean change from baseline to Week 12 for sham eyes in the central subfield of the outer retina (difference of −0.003 mm³; P=0.035), and in the central and inferior subfield of the photoreceptor layer (difference of −0.002 mm³; P=0.009 and difference of −0.002 mm³; P=0.041, respectively). In most of these instances, the risuteganib eyes had the larger decrease in thickness or volume over time, with the sham eyes showing a smaller decrease or an increase in measurement; however, the sham eyes had a larger decrease in mean thickness in the foveal center of the inner retina.

No other anatomical measurements showed a significant difference between risuteganib and sham eyes over time.

### OCT Analysis 2:

In Analysis #2, the OCT images of study eyes were analyzed to determine mean thickness and mean volume of numerous retinal subfields and layer segments at baseline and at Week 12 for sham eyes and at baseline and at Week 32 for risuteganib eyes, to document any significant differences between groups of eyes based on baseline measurements or changes from baseline in those measurements.

Anatomical Measurements at Baseline by Risuteganib Responder Status. At baseline, those eyes that responded to risuteganib had significantly greater mean thickness in 7 different retinal metrics compared with eyes that did not respond to risuteganib: mean total retinal central subfield thickness (256.11 vs 221.13 μm; P=0.011), mean total retinal mid subfield (central 2 mm) thickness (294.80 vs 265.73 μm; P=0.004), mean ONL-RPE fovea thickness (170.66 vs 136.07 μm; P=0.020), mean ONL-RPE central subfield thickness (149.43 vs 123.33 μm; P=0.003), mean ONL-RPE mid subfield thickness (130.07 vs 112.01 μm; P=0.023), mean ONL-EZ central subfield thickness (116.17 vs 101.31 μm; P=0.021), and mean ONL-EZ mid subfield thickness (95.43 vs 86.15 μm; P=0.032) These data are summarized in Table 16, below:

TABLE 16
Quantitative Anatomical Measurements at Baseline for
Risuteganib Nonresponder Eyes Versus Responder Eyes

| | Risuteganib | | Risuteganib | | Two-Sample |
|---|---|---|---|---|---|
| Measurement | Nonresponder | | Responder | | T-test |
| Sector | n = 13 | | n = 12 | | P-value |
| Mean (SD) thickness, μm | | | | | |
| Total retinal foveal center | 177.80 | (44.98) | 204.31 | (26.95) | 0.087 |
| Total retinal central subfield | 221.13 | (32.68) | 256.11 | (30.19) | 0.011 |
| Total retinal mid subfield | 265.73 | (22.06) | 294.80 | (23.81) | 0.004 |
| EZ-RPE foveal center | 19.95 | (26.67) | 38.67 | (25.52) | 0.086 |
| EZ-RPE central subfield | 22.02 | (16.18) | 33.26 | (12.70) | 0.065 |
| EZ-RPE mid subfield | 25.86 | (14.79) | 34.63 | (10.94) | 0.104 |
| ONL-RPE foveal center | 136.07 | (42.38) | 170.66 | (23.56) | 0.020 |
| ONL-RPE central subfield | 123.33 | (21.74) | 149.43 | (17.71) | 0.003 |
| ONL-RPE mid subfield | 112.01 | (21.78) | 130.07 | (14.51) | 0.023 |
| RPE-BM foveal center | 34.21 | (33.57) | 47.62 | (51.59) | 0.455 |
| RPE-BM central subfield | 34.89 | (22.02) | 42.64 | (39.54) | 0.557 |
| RPE-BM mid subfield | 29.53 | (17.97) | 36.52 | (24.24) | 0.425 |
| ELM-RPE foveal center | 42.76 | (33.22) | 56.87 | (33.57) | 0.302 |
| ELM-RPE central subfield | 41.24 | (22.07) | 57.56 | (20.41) | 0.067 |
| ELM-RPE mid subfield | 43.73 | (21.14) | 57.05 | (17.35) | 0.098 |
| Inner retina central subfield | 97.80 | (21.56) | 106.68 | (19.28) | 0.288 |
| Inner retina mid subfield | 153.72 | (16.04) | 164.73 | (19.51) | 0.139 |
| ELM-EZ central subfield | 19.22 | (8.93) | 24.30 | (8.28) | 0.154 |
| ELM-EZ mid subfield | 17.88 | (7.14) | 22.42 | (6.99) | 0.122 |
| ONL-EZ central subfield | 101.31 | (16.52) | 116.17 | (13.26) | 0.021 |
| ONL-EZ mid subfield | 86.15 | (10.71) | 95.43 | (9.57) | 0.032 |
| Volume, mm³ | | | | | |
| Total retinal | 9.40 | (0.51) | 9.87 | (0.75) | 0.081 |
| Total retinal central subfield | 0.17 | (0.03) | 0.20 | (0.02) | 0.010 |
| Total retinal mid subfield | 0.83 | (0.07) | 0.93 | (0.07) | 0.004 |
| EZ-RPE | 1.28 | (0.33) | 1.33 | (0.27) | 0.636 |
| EZ-RPE central subfield | 0.02 | (0.01) | 0.03 | (0.01) | 0.063 |
| EZ-RPE mid subfield | 0.08 | (0.05) | 0.11 | (0.03) | 0.102 |
| ONL-RPE | 3.78 | (0.49) | 4.09 | (0.30) | 0.070 |
| ONL-RPE central subfield | 0.10 | (0.02) | 0.12 | (0.01) | 0.003 |
| ONL-RPE mid subfield | 0.35 | (0.07) | 0.41 | (0.05) | 0.022 |
| RPE-BM | 0.55 | (0.15) | 0.63 | (0.14) | 0.192 |
| RPE-BM central subfield | 0.03 | (0.02) | 0.03 | (0.03) | 0.551 |
| RPE-BM mid subfield | 0.09 | (0.06) | 0.11 | (0.08) | 0.421 |
| ELM-RPE | 3.07 | (0.46) | 3.33 | (0.30) | 0.100 |
| ELM-RPE central subfield | 0.03 | (0.02) | 0.05 | (0.02) | 0.066 |
| ELM-RPE mid subfield | 0.14 | (0.07) | 0.18 | (0.05) | 0.096 |
| ELM-EZ central subfield | 0.02 | (0.01) | 0.02 | (0.01) | 0.155 |
| ELM-EZ mid subfield | 0.06 | (0.02) | 0.07 | (0.02) | 0.121 |
| ONL-EZ central subfield | 0.08 | (0.01) | 0.09 | (0.01) | 0.021 |
| ONL-EZ mid subfield | 0.27 | (0.03) | 0.30 | (0.03) | 0.030 |
| Map coverage, % | | | | | |
| 250 μm RPE-BM | 0.00 | (0.00) | 0.01 | (0.04) | 0.339 |
| 150 μm RPE-BM | 0.30 | (0.64) | 0.26 | (0.82) | 0.888 |
| 50 μm RPE-BM | 1.99 | (3.63) | 3.54 | (3.60) | 0.296 |
| 0 μm RPE-BM | 9.06 | (14.78) | 7.33 | (14.43) | 0.770 |
| 20 μm EZ | 7.01 | (12.39) | 5.76 | (12.81) | 0.806 |
| 10 μm EZ | 6.71 | (12.36) | 5.49 | (12.47) | 0.808 |
| 0 μm EZ | 1.76 | (5.60) | 1.29 | (3.64) | 0.806 |

Abbreviations: ELM-EZ, external limiting membrane-ellipsoid zone; ELM-RPE, external limiting membrane-retinal pigment epithelium; EZ, ellipsoid zone; EZ-RPE, ellipsoid zone-retinal pigment epithelium; ONL-EZ, outer nuclear layer-ellipsoid zone; ONL-RPE, outer nuclear layer-retinal pigment epithelium; RPE-BM, retinal pigment epithelium-Bruch's membrane.

Six of the same 7 metrics in risuteganib responder eyes also had significantly greater volume at baseline compared with risuteganib nonresponder eyes: total retinal central subfield volume (0.20 vs 0.17 mm³; P=0.010), total retinal mid subfield volume (0.93 vs 0.83 mm³; P=0.004), ONL-RPE central subfield volume (0.12 vs 0.10 mm³; P=0.003), ONL-RPE mid subfield volume (0.41 vs 0.35 mm³; P=0.022), ONL-EZ central subfield volume (0.09 vs 0.08 mm³; P=0.021), and ONL-EZ mid subfield volume (0.30 vs 0.27 mm³; P=0.030).

No other anatomical measurements showed a significant difference between responder and nonresponder eyes at baseline.

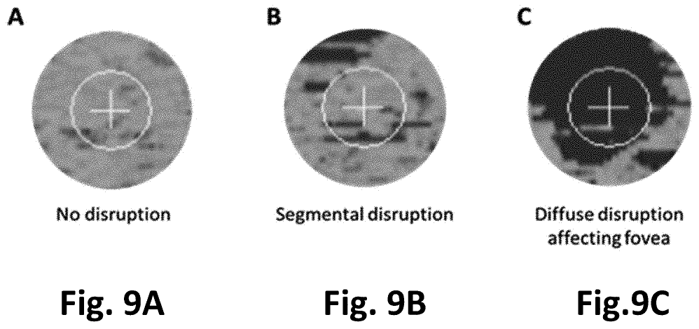

In addition to the quantitative analysis of OCT images, OCT Analysis #2 included qualitative assessment of the OCT images to identify GA, pseudodrusen, and disruption of the ELM and EZ layers. FIGS. 9A, 9B and 9C illustrate the level of varying pathology within the ELM based on quantitative mapping that were also assessed, with FIG. 9A (left) showing no ELM disruption, FIG. 9B (center) showing segmental disruption, and FIG. 9C showing diffuse disruption.

Qualitative assessment revealed no significant differences in anatomical features at baseline between risuteganib responder and nonresponder eyes, with the exception of diffuse disruption of the central 1-mm quadrant of the EZ layer (P=0.027).

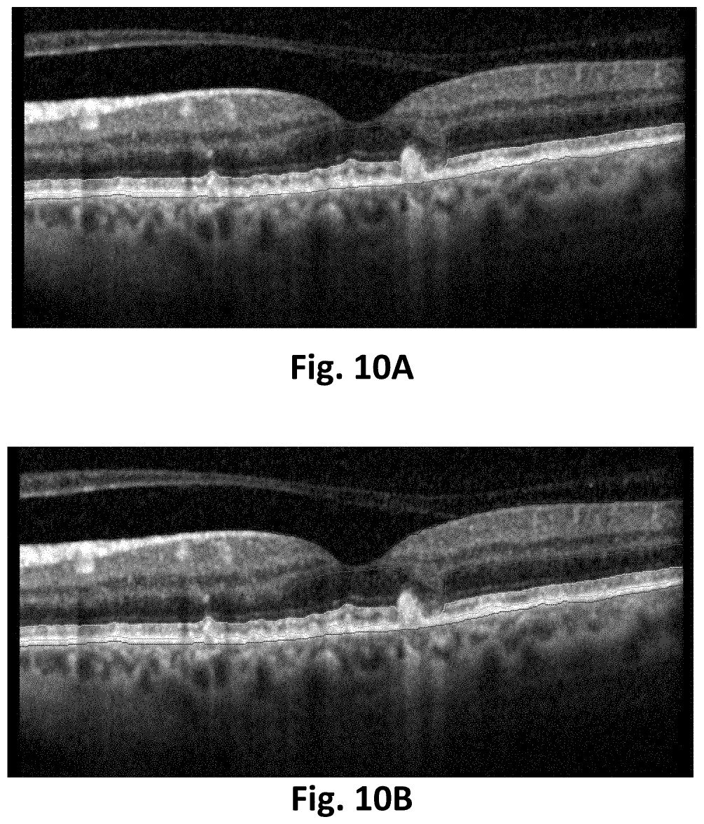

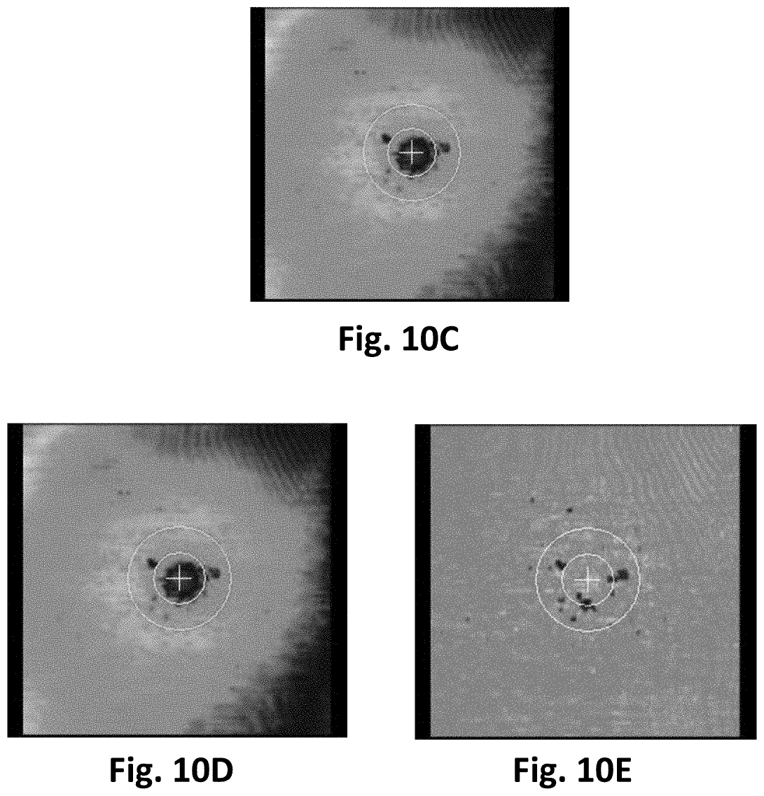

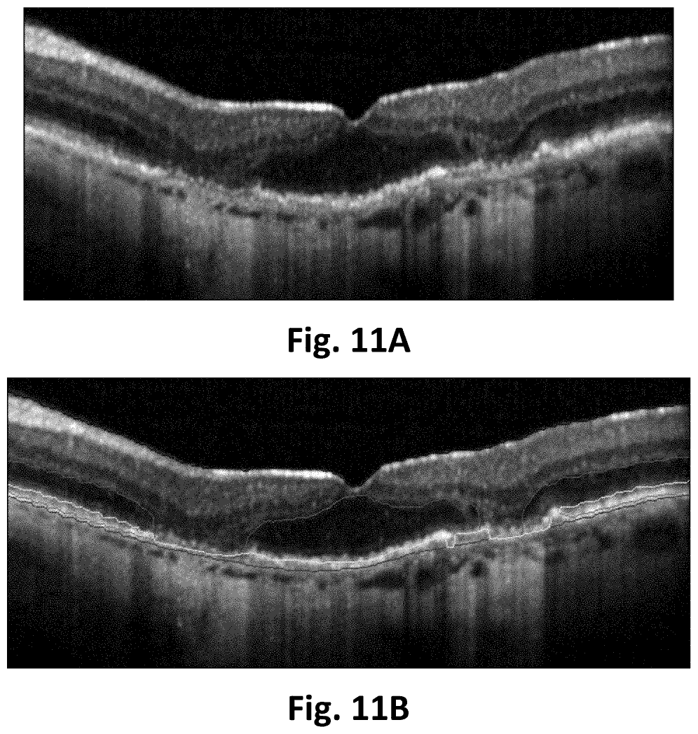

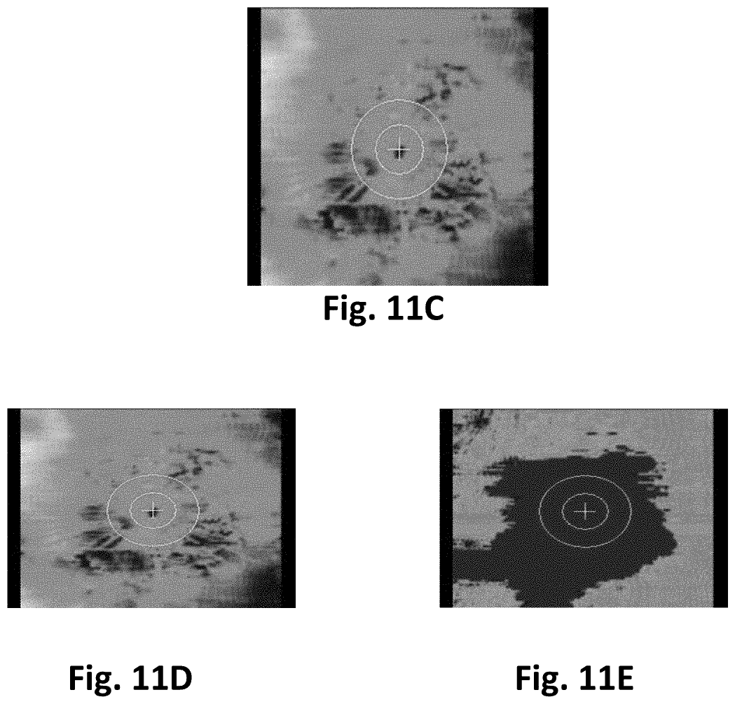

FIGS. 10A through 10E and FIGS. 11A through 11E show OCT and map images at baseline of a risuteganib responder eye and nonresponder eye, respectively. Both ILM-RPE maps (FIGS. 10C and 11C) eveal primarily normal images. However, the risuteganib responder eye shows only small areas of attenuation/atrophy in the EZ-RPE map of FIG. 10D and the RPE-BM map of FIG. 10D while the non-responder eye shows diffuse attenuation/atrophy in the EZ-RPE map of FIG. 11D and the RPE-BM map of FIG. 11D.

Anatomical Measurements at Baseline by Risuteganib Responder Status. At baseline, the eight (8) study eyes that responded to risuteganib with an improvement of at least 11 letters (referred to below as “super-responders”) had significantly greater mean thickness in 7 different retinal metrics compared with risuteganib nonresponder eyes: mean total retinal central subfield thickness (255.74 vs 221.13 μm; P=0.046), mean total retinal mid subfield thickness (293.59 vs 265.73 μm; P=0.021), mean ONL-RPE fovea thickness (167.75 vs 136.07 μm; P=0.044), mean ONL-RPE central subfield thickness (150.31 vs 123.33 μm; P=0.014), mean ONL-RPE mid subfield thickness (130.85 vs 112.01 μm; P=0.040), mean ONL-EZ central subfield thickness (117.93 vs 101.31 μm; P=0.023), and mean ONL-EZ mid subfield thickness (97.92 vs 86.15 μm; P=0.010) These data are summarized in Table 17, below:

TABLE 17
Quantitative Anatomical Measurements at Baseline for Risuteganib
Nonresponder Eyes Versus Super-Responder Eyes

| | Risuteganib | | Risuteganib | | Two-Sample |
|---|---|---|---|---|---|
| Measurement | Nonresponder | | Super-Responder | | T-test |
| Sector | n = 13 | | n = 8 | | P-value |
| Mean (SD) thickness, μm | | | | | |
| Total retinal foveal center | 177.80 | (44.98) | 204.09 | (29.78) | 0.124 |
| Total retinal central subfield | 221.13 | (32.68) | 255.74 | (36.57) | 0.046 |
| Total retinal mid subfield | 265.73 | (22.06) | 293.59 | (24.86) | 0.021 |
| EZ-RPE foveal center | 19.95 | (26.67) | 32.66 | (24.75) | 0.284 |
| EZ-RPE central subfield | 22.02 | (16.18) | 32.38 | (15.50) | 0.163 |
| EZ-RPE mid subfield | 25.86 | (14.79) | 32.94 | (13.16) | 0.270 |
| ONL-RPE foveal center | 136.07 | (42.38) | 167.75 | (24.90) | 0.044 |
| ONL-RPE central subfield | 123.33 | (21.74) | 150.31 | (21.35) | 0.014 |
| ONL-RPE mid subfield | 112.01 | (21.78) | 130.85 | (16.91) | 0.040 |
| RPE-BM foveal center | 34.21 | (33.57) | 52.90 | (61.27) | 0.447 |
| RPE-BM central subfield | 34.89 | (22.02) | 42.44 | (47.21) | 0.681 |
| RPE-BM mid subfield | 29.53 | (17.97) | 36.07 | (28.87) | 0.577 |
| ELM-RPE foveal center | 42.76 | (33.22) | 54.11 | (36.01) | 0.482 |
| ELM-RPE central subfield | 41.24 | (22.07) | 54.52 | (24.75) | 0.235 |
| ELM-RPE mid subfield | 43.73 | (21.14) | 53.96 | (20.87) | 0.295 |
| Inner retina central subfield | 97.80 | (21.56) | 105.43 | (23.09) | 0.463 |
| Inner retina mid subfield | 153.72 | (16.04) | 162.73 | (19.77) | 0.297 |
| ELM-EZ central subfield | 19.22 | (8.93) | 22.14 | (9.52) | 0.497 |
| ELM-EZ mid subfield | 17.88 | (7.14) | 21.02 | (8.36) | 0.393 |
| ONL-EZ central subfield | 101.31 | (16.52) | 117.93 | (13.73) | 0.023 |
| ONL-EZ mid subfield | 86.15 | (10.71) | 97.92 | (7.96) | 0.010 |
| Volume, mm³ | | | | | |
| Total retinal | 9.40 | (0.51) | 9.88 | (0.60) | 0.080 |
| Total retinal central subfield | 0.17 | (0.03) | 0.20 | (0.03) | 0.045 |
| Total retinal mid subfield | 0.83 | (0.07) | 0.92 | (0.08) | 0.021 |
| EZ-RPE | 1.28 | (0.33) | 1.30 | (0.33) | 0.888 |
| EZ-RPE central subfield | 0.02 | (0.01) | 0.03 | (0.01) | 0.160 |
| EZ-RPE mid subfield | 0.08 | (0.05) | 0.10 | (0.04) | 0.268 |
| ONL-RPE | 3.78 | (0.49) | 4.13 | (0.32) | 0.069 |
| ONL-RPE central subfield | 0.10 | (0.02) | 0.12 | (0.02) | 0.013 |
| ONL-RPE mid subfield | 0.35 | (0.07) | 0.41 | (0.05) | 0.039 |
| RPE-BM | 0.55 | (0.15) | 0.61 | (0.16) | 0.407 |
| RPE-BM central subfield | 0.03 | (0.02) | 0.03 | (0.04) | 0.675 |
| RPE-BM mid subfield | 0.09 | (0.06) | 0.11 | (0.09) | 0.574 |
| ELM-RPE | 3.07 | (0.46) | 3.33 | (0.35) | 0.152 |
| ELM-RPE central subfield | 0.03 | (0.02) | 0.04 | (0.02) | 0.232 |
| ELM-RPE mid subfield | 0.14 | (0.07) | 0.17 | (0.07) | 0.294 |
| ELM-EZ central subfield | 0.02 | (0.01) | 0.02 | (0.01) | 0.499 |
| ELM-EZ mid subfield | 0.06 | (0.02) | 0.07 | (0.03) | 0.392 |
| ONL-EZ central subfield | 0.08 | (0.01) | 0.09 | (0.01) | 0.023 |
| ONL-EZ mid subfield | 0.27 | (0.03) | 0.31 | (0.03) | 0.010 |
| Map coverage, % | | | | | |
| 250 μm RPE-BM | 0.00 | (0.00) | 0.02 | (0.05) | 0.351 |
| 150 μm RPE-BM | 0.30 | (0.64) | 0.39 | (1.00) | 0.829 |
| 50 μm RPE-BM | 1.99 | (3.63) | 3.29 | (4.18) | 0.481 |
| 0 μm RPE-BM | 9.06 | (14.78) | 9.96 | (17.39) | 0.905 |
| 20 μm EZ | 7.01 | (12.39) | 8.08 | (15.45) | 0.871 |
| 10 μm EZ | 6.71 | (12.36) | 7.77 | (15.03) | 0.870 |
| 0 μm EZ | 1.76 | (5.60) | 1.93 | (4.41) | 0.940 |

Abbreviations: ELM-EZ, external limiting membrane-ellipsoid zone; ELM-RPE, external limiting membrane-retinal pigment epithelium; EZ, ellipsoid zone; EZ-RPE, ellipsoid zone-retinal pigment epithelium; ONL-EZ, outer nuclear layer-ellipsoid zone; ONL-RPE, outer nuclear layer-retinal pigment epithelium; RPE-BM, retinal pigment epithelium-Bruch's membrane.

Six of the same 7 metrics in super-responder eyes also had significantly greater volume at baseline compared with nonresponder eyes: total retinal central subfield volume (0.20 vs 0.17 mm³; P=0.045), total retinal mid subfield volume (0.92 vs 0.83 mm³; P=0.021), ONL-RPE central subfield volume (0.12 vs 0.10 mm³; P=0.013), ONL-RPE mid subfield volume (0.41 vs 0.35 mm³; P=0.039), ONL-EZ central subfield volume (0.09 vs 0.08 mm³; P=0.023), and ONL-EZ mid subfield volume (0.31 vs 0.27 mm³; P=0.010). Apart from these noted differences in volume, no significant differences in anatomical features at baseline were observed between risuteganib super-responder and nonresponder eyes, as shown in Table 17 above.

No other anatomical measurements, including map coverage, showed a significant difference between super-responder and nonresponder eyes at baseline.

Anatomical Measurements at Baseline of Risuteganib Subgroups vs Sham Arm. At baseline, no anatomical measurements showed a significant difference between risuteganib nonresponder eyes and sham eyes. This is summarized in Table 18, below:

TABLE 18
Quantitative Anatomical Measurements at Baseline for
Risuteganib Nonresponder Eyes Versus Sham Eyes

| | Risuteganib | | | | Two-Sample |
|---|---|---|---|---|---|
| Measurement | Nonresponder | | Sham | | T-test |
| Sector | n = 13 | | n = 14 | | P-value |
| Mean (SD) thickness, μm | | | | | |
| Total retinal foveal center | 177.80 | (44.98) | 167.20 | (54.25) | 0.585 |
| Total retinal central subfield | 221.13 | (32.68) | 235.46 | (32.19) | 0.262 |
| Total retinal mid subfield | 265.73 | (22.06) | 276.31 | (29.47) | 0.299 |
| EZ-RPE foveal center | 19.95 | (26.67) | 27.03 | (22.25) | 0.463 |
| EZ-RPE central subfield | 22.02 | (16.18) | 27.06 | (15.74) | 0.420 |
| EZ-RPE mid subfield | 25.86 | (14.79) | 26.73 | (14.62) | 0.878 |
| ONL-RPE foveal center | 136.07 | (42.38) | 141.01 | (49.01) | 0.781 |
| ONL-RPE central subfield | 123.33 | (21.74) | 130.54 | (32.27) | 0.500 |
| ONL-RPE mid subfield | 112.01 | (21.78) | 111.31 | (35.19) | 0.951 |
| RPE-BM foveal center | 34.21 | (33.57) | 50.85 | (46.64) | 0.296 |
| RPE-BM central subfield | 34.89 | (22.02) | 40.17 | (30.47) | 0.609 |
| RPE-BM mid subfield | 29.53 | (17.97) | 35.31 | (35.03) | 0.592 |
| ELM-RPE foveal center | 42.76 | (33.22) | 48.62 | (34.85) | 0.658 |
| ELM-RPE central subfield | 41.24 | (22.07) | 49.58 | (21.45) | 0.330 |
| ELM-RPE mid subfield | 43.73 | (21.14) | 47.86 | (20.08) | 0.608 |
| Inner retina central subfield | 97.80 | (21.56) | 104.92 | (23.12) | 0.416 |
| Inner retina mid subfield | 153.72 | (16.04) | 165.00 | (19.67) | 0.114 |
| ELM-EZ central subfield | 19.22 | (8.93) | 22.52 | (8.46) | 0.336 |
| ELM-EZ mid subfield | 17.88 | (7.14) | 21.13 | (7.79) | 0.268 |
| ONL-EZ central subfield | 101.31 | (16.52) | 103.48 | (20.40) | 0.764 |
| ONL-EZ mid subfield | 86.15 | (10.71) | 35.48 | (22.53) | 0.817 |
| Volume, mm³ | | | | | |
| Total retinal | 9.40 | (0.51) | 9.78 | (1.05) | 0.240 |
| Total retinal central subfield | 0.17 | (0.03) | 0.18 | (0.02) | 0.283 |
| Total retinal mid subfield | 0.83 | (0.07) | 0.87 | (0.09) | 0.293 |
| EZ-RPE | 1.28 | (0.33) | 1.18 | (0.34) | 0.489 |
| EZ-RPE central subfield | 0.02 | (0.01) | 0.02 | (0.01) | 0.422 |
| EZ-RPE mid subfield | 0.08 | (0.05) | 0.08 | (0.05) | 0.878 |
| ONL-RPE | 3.78 | (0.49) | 3.86 | (0.52) | 0.705 |
| ONL-RPE central subfield | 0.10 | (0.02) | 0.10 | (0.02) | 0.519 |
| ONL-RPE mid subfield | 0.35 | (0.07) | 0.35 | (0.11) | 0.952 |
| RPE-BM | 0.55 | (0.15) | 0.74 | (0.33) | 0.062 |
| RPE-BM central subfield | 0.03 | (0.02) | 0.03 | (0.02) | 0.611 |
| RPE-BM mid subfield | 0.09 | (0.06) | 0.11 | (0.11) | 0.590 |
| ELM-RPE | 3.07 | (0.46) | 3.14 | (0.42) | 0.686 |
| ELM-RPE central subfield | 0.03 | (0.02) | 0.04 | (0.02) | 0.334 |
| ELM-RPE mid subfield | 0.14 | (0.07) | 0.15 | (0.06) | 0.608 |
| ELM-EZ central subfield | 0.02 | (0.01) | 0.02 | (0.01) | 0.346 |
| ELM-EZ mid subfield | 0.06 | (0.02) | 0.07 | (0.02) | 0.269 |
| ONL-EZ central subfield | 0.08 | (0.01) | 0.08 | (0.02) | 0.800 |
| ONL-EZ mid subfield | 0.27 | (0.03) | 0.27 | (0.07) | 0.819 |
| Map coverage, % | | | | | |
| 250 μm RPE-BM | 0.00 | (0.00) | 0.17 | (0.63) | 0.336 |
| 150 μm RPE-BM | 0.30 | (0.64) | 1.77 | (3.47) | 0.144 |
| 50 μm RPE-BM | 1.99 | (3.63) | 3.99 | (6.59) | 0.337 |
| 0 μm RPE-BM | 9.06 | (14.78) | 14.40 | (20.12) | 0.437 |
| 20 μm EZ | 7.01 | (12.39) | 12.39 | (19.63) | 0.400 |
| 10 μm EZ | 6.71 | (12.36) | 11.99 | (19.55) | 0.408 |
| 0 μm EZ | 1.76 | (5.60) | 1.46 | (3.45) | 0.870 |

Abbreviations: ELM-EZ, external limiting membrane-ellipsoid zone; ELM-RPE, external limiting membrane-retinal pigment epithelium; EZ, ellipsoid zone; EZ-RPE, ellipsoid zone-retinal pigment epithelium; ONL-EZ, outer nuclear layer-ellipsoid zone; ONL-RPE, outer nuclear layer-retinal pigment epithelium; RPE-BM, retinal pigment epithelium-Bruch's membrane.

Compared with sham eyes, risuteganib responder eyes had significantly greater mean thickness in the total retinal foveal center at baseline (204.31 vs 167.20 μm; P=0.036). This is summarized in the following Table 19. No other anatomical measurements showed a significant difference between risuteganib responder eyes and sham eyes at baseline.

TABLE 19
Quantitative Anatomical Measurements at Baseline
for Risuteganib Responder Eyes Versus Sham Eyes

| | Risuteganib | | | | Two-Sample |
|---|---|---|---|---|---|
| Measurement | Responder | | Sham | | T-test |
| Sector | n = 12 | | n = 14 | | P-value |
| Mean (SD) thickness, μm | | | | | |
| Total retinal foveal center | 204.31 | (26.95) | 167.20 | (54.25) | 0.036 |
| Total retinal central subfield | 256.11 | (30.19) | 235.46 | (32.19) | 0.105 |
| Total retinal mid subfield | 294.80 | (23.81) | 276.31 | (29.47) | 0.090 |
| EZ-RPE foveal center | 38.67 | (25.52) | 27.03 | (22.25) | 0.232 |
| EZ-RPE central subfield | 33.26 | (12.70) | 27.06 | (15.74) | 0.278 |
| EZ-RPE mid subfield | 34.63 | (10.94) | 26.73 | (14.62) | 0.129 |
| ONL-RPE foveal center | 170.66 | (23.56) | 141.01 | (49.01) | 0.059 |
| ONL-RPE central subfield | 149.43 | (17.71) | 130.54 | (32.27) | 0.074 |
| ONL-RPE mid subfield | 130.07 | (14.51) | 111.31 | (35.19) | 0.085 |
| RPE-BM foveal center | 47.62 | (51.59) | 50.85 | (46.64) | 0.869 |
| RPE-BM central subfield | 42.64 | (39.54) | 40.17 | (30.47) | 0.862 |
| RPE-BM mid subfield | 36.52 | (24.24) | 35.31 | (35.03) | 0.918 |
| ELM-RPE foveal center | 56.87 | (33.57) | 48.62 | (34.85) | 0.545 |
| ELM-RPE central subfield | 57.56 | (20.41) | 49.58 | (21.45) | 0.341 |
| ELM-RPE mid subfield | 57.05 | (17.35) | 47.86 | (20.08) | 0.223 |
| Inner retina central subfield | 106.68 | (19.28) | 104.92 | (23.12) | 0.834 |
| Inner retina mid subfield | 164.73 | (19.51) | 165.00 | (19.67) | 0.973 |
| ELM-EZ central subfield | 24.30 | (8.28) | 22.52 | (8.46) | 0.592 |
| ELM-EZ mid subfield | 22.42 | (6.99) | 21.13 | (7.79) | 0.661 |
| ONL-EZ central subfield | 116.17 | (13.26) | 103.48 | (20.40) | 0.070 |
| ONL-EZ mid subfield | 95.43 | (9.57) | 35.48 | (22.53) | 0.119 |
| Volume, mm³ | | | | | |
| Total retinal | 9.87 | (0.75) | 9.78 | (1.05) | 0.787 |
| Total retinal central subfield | 0.20 | (0.02) | 0.18 | (0.02) | 0.089 |
| Total retinal mid subfield | 0.93 | (0.07) | 0.87 | (0.09) | 0.084 |
| EZ-RPE | 1.33 | (0.27) | 1.18 | (0.34) | 0.228 |
| EZ-RPE central subfield | 0.03 | (0.01) | 0.02 | (0.01) | 0.265 |
| EZ-RPE mid subfield | 0.11 | (0.03) | 0.08 | (0.05) | 0.127 |
| ONL-RPE | 4.09 | (0.30) | 3.86 | (0.52) | 0.166 |
| ONL-RPE central subfield | 0.12 | (0.01) | 0.10 | (0.02) | 0.064 |
| ONL-RPE mid subfield | 0.41 | (0.05) | 0.35 | (0.11) | 0.083 |
| RPE-BM | 0.63 | (0.14) | 0.74 | (0.33) | 0.249 |
| RPE-BM central subfield | 0.03 | (0.03) | 0.03 | (0.02) | 0.849 |
| RPE-BM mid subfield | 0.11 | (0.08) | 0.11 | (0.11) | 0.915 |
| ELM-RPE | 3.33 | (0.30) | 3.14 | (0.42) | 0.186 |
| ELM-RPE central subfield | 0.05 | (0.02) | 0.04 | (0.02) | 0.327 |
| ELM-RPE mid subfield | 0.18 | (0.05) | 0.15 | (0.06) | 0.219 |
| ELM-EZ central subfield | 0.02 | (0.01) | 0.02 | (0.01) | 0.579 |
| ELM-EZ mid subfield | 0.07 | (0.02) | 0.07 | (0.02) | 0.655 |
| ONL-EZ central subfield | 0.09 | (0.01) | 0.08 | (0.02) | 0.061 |
| ONL-EZ mid subfield | 0.30 | (0.03) | 0.27 | (0.07) | 0.115 |
| Map coverage, % | | | | | |
| 250 μm RPE-BM | 0.01 | (0.04) | 0.17 | (0.63) | 0.369 |
| 150 μm RPE-BM | 0.26 | (0.82) | 1.77 | (3.47) | 0.137 |
| 50 μm RPE-BM | 3.54 | (3.60) | 3.99 | (6.59) | 0.829 |
| 0 μm RPE-BM | 7.33 | (14.43) | 14.40 | (20.12) | 0.309 |
| 20 μm EZ | 5.76 | (12.81) | 12.39 | (19.63) | 0.312 |
| 10 μm EZ | 5.49 | (12.47) | 11.99 | (19.55) | 0.317 |
| 0 μm EZ | 1.29 | (3.64) | 1.46 | (3.45) | 0.906 |

Abbreviations: ELM-EZ, external limiting membrane-ellipsoid zone; ELM-RPE, external limiting membrane-retinal pigment epithelium; EZ, ellipsoid zone; EZ-RPE, ellipsoid zone-retinal pigment epithelium; ONL-EZ, outer nuclear layer-ellipsoid zone; ONL-RPE, outer nuclear layer-retinal pigment epithelium; RPE-BM, retinal pigment epithelium-Bruch's membrane.

Anatomical Measurements at Baseline by Treatment Arm. At baseline, no anatomical measurements showed a significant difference between the risuteganib arm and the sham arm. This is summarized in Table 20, below.

TABLE 20
Quantitative Anatomical Measurements at Baseline
for Risuteganib Arm Versus Sham Arm

| | | | | | Two-Sample |
|---|---|---|---|---|---|
| Measurement | Risuteganib | | Sham | | T-test |
| Sector | n = 25 | | n = 14 | | P-value |
| Mean (SD) thickness, μm | | | | | |
| Total retinal foveal center (fovea) | 190.53 | (39.08) | 167.20 | (54.25) | 0.172 |
| Total retinal central subfield | 237.92 | (35.64) | 235.46 | (32.19) | 0.827 |
| Total retinal mid subfield | 279.68 | (26.89) | 276.31 | (29.47) | 0.726 |
| EZ-RPE foveal center (fovea) | 28.94 | (27.30) | 27.03 | (22.25) | 0.814 |
| EZ-RPE central subfield | 27.41 | (15.42) | 27.06 | (15.74) | 0.947 |
| EZ-RPE mid subfield | 30.07 | (13.57) | 26.73 | (14.62) | 0.489 |
| ONL-RPE foveal center | 152.67 | (38.26) | 141.01 | (49.01) | 0.450 |
| ONL-RPE central subfield | 135.86 | (23.61) | 130.54 | (32.27) | 0.595 |
| ONL-RPE mid subfield | 120.68 | (20.46) | 111.31 | (35.19) | 0.373 |
| RPE-BM foveal center | 40.65 | (42.78) | 50.85 | (46.64) | 0.506 |
| RPE-BM central subfield | 38.61 | (31.22) | 40.17 | (30.47) | 0.880 |
| RPE-BM mid subfield | 32.89 | (21.06) | 35.31 | (35.03) | 0.816 |
| ELM-RPE foveal center | 49.54 | (33.47) | 48.62 | (34.85) | 0.937 |
| ELM-RPE central subfield | 49.07 | (22.45) | 49.58 | (21.45) | 0.945 |
| ELM-RPE mid subfield | 50.13 | (20.19) | 47.86 | (20.08) | 0.739 |
| Inner retina central subfield | 102.06 | (20.57) | 104.92 | (23.12) | 0.704 |
| Inner retina mid subfield | 159.01 | (18.29) | 165.00 | (19.67) | 0.358 |
| ELM-EZ central subfield | 21.66 | (8.83) | 22.52 | (8.46) | 0.768 |
| ELM-EZ mid subfield | 20.06 | (7.30) | 21.13 | (7.79) | 0.676 |
| ONL-EZ central subfield | 108.44 | (16.57) | 103.48 | (20.40) | 0.445 |
| ONL-EZ mid subfield | 90.61 | (11.03) | 35.48 | (22.53) | 0.361 |
| Volume, mm³ | | | | | |
| Total retinal | 9.63 | (0.67) | 9.78 | (1.05) | 0.632 |
| Total retinal central subfield | 0.19 | (0.03) | 0.18 | (0.02) | 0.771 |
| Total retinal mid subfield | 0.88 | (0.08) | 0.87 | (0.09) | 0.718 |
| EZ-RPE | 1.30 | (0.30) | 1.18 | (0.34) | 0.286 |
| EZ-RPE central subfield | 0.02 | (0.01) | 0.02 | (0.01) | 0.932 |
| EZ-RPE mid subfield | 0.09 | (0.04) | 0.08 | (0.05) | 0.487 |
| ONL-RPE | 3.93 | (0.43) | 3.86 | (0.52) | 0.655 |
| ONL-RPE central subfield | 0.11 | (0.02) | 0.10 | (0.02) | 0.559 |
| ONL-RPE mid subfield | 0.38 | (0.06) | 0.35 | (0.11) | 0.369 |
| RPE-BM | 0.59 | (0.15) | 0.74 | (0.33) | 0.112 |
| RPE-BM central subfield | 0.03 | (0.02) | 0.03 | (0.02) | 0.892 |
| RPE-BM mid subfield | 0.10 | (0.07) | 0.11 | (0.11) | 0.817 |
| ELM-RPE | 3.20 | (0.40) | 3.14 | (0.42) | 0.685 |
| ELM-RPE central subfield | 0.04 | (0.02) | 0.04 | (0.02) | 0.962 |
| ELM-RPE mid subfield | 0.16 | (0.06) | 0.15 | (0.06) | 0.735 |
| ELM-EZ central subfield | 0.02 | (0.01) | 0.02 | (0.01) | 0.784 |
| ELM-EZ mid subfield | 0.06 | (0.02) | 0.07 | (0.02) | 0.680 |
| ONL-EZ central subfield | 0.09 | (0.01) | 0.08 | (0.02) | 0.409 |
| ONL-EZ mid subfield | 0.28 | (0.03) | 0.27 | (0.07) | 0.356 |
| Map coverage, % | | | | | |
| 250 μm RPE-BM | 0.01 | (0.03) | 0.17 | (0.63) | 0.351 |
| 150 μm RPE-BM | 0.28 | (0.72) | 1.77 | (3.47) | 0.137 |
| 50 μm RPE-BM | 2.73 | (3.63) | 3.99 | (6.59) | 0.519 |
| 0 μm RPE-BM | 8.23 | (14.33) | 14.40 | (20.12) | 0.323 |
| 20 μm EZ | 6.41 | (12.34) | 12.39 | (19.63) | 0.315 |
| 10 μm EZ | 6.12 | (12.17) | 11.99 | (19.55) | 0.322 |
| 0 μm EZ | 1.53 | (4.67) | 1.46 | (3.45) | 0.955 |

Abbreviations: ELM-EZ, external limiting membrane-ellipsoid zone; ELM-RPE, external limiting membrane-retinal pigment epithelium; EZ, ellipsoid zone; EZ-RPE, ellipsoid zone-retinal pigment epithelium; ONL-EZ, outer nuclear layer-ellipsoid zone; ONL-RPE, outer nuclear layer-retinal pigment epithelium; RPE-BM, retinal pigment epithelium-Bruch's membrane.

No anatomical measurements showed a significant difference in the change from baseline at Week 32 between risuteganib responder eyes and nonresponder eyes, except for the change in RPE-BM volume (−0.049 vs 0.037 mm³; P=0.034), with the responder eyes showing a decline and the nonresponder eyes showing an increase, as summarized in Table 21, below:

TABLE 21
Quantitative Anatomical Measurements Change From Baseline at Week
32 for Risuteganib Nonresponder Eyes Versus Responder Eyes

| | Risuteganib | | Risuteganib | | Two-Sample |
|---|---|---|---|---|---|
| Measurement | Nonresponder | | Responder | | T-test |
| Sector | n = 12 | | n = 12 | | P-value |
| Change in mean (SD) thickness, μm | | | | | |
| Total retinal foveal center | −9.112 | (37.435) | 0.804 | (32.231) | 0.494 |
| Total retinal central subfield | −5.981 | (10.604) | −0.691 | (10.370) | 0.230 |
| Total retinal mid subfield | −4.046 | (5.084) | −1.049 | (6.183) | 0.209 |
| EZ-RPE foveal center | −1.789 | (30.522) | 0.975 | (21.174) | 0.799 |
| EZ-RPE central subfield | −1.390 | (6.069) | −0.779 | (3.229) | 0.762 |
| EZ-RPE mid subfield | −1.798 | (3.956) | −1.174 | (3.772) | 0.696 |
| ONL-RPE foveal center | −7.626 | (40.364) | 0.650 | (30.970) | 0.579 |
| ONL-RPE central subfield | −7.877 | (14.446) | −6.555 | (15.778) | 0.832 |
| ONL-RPE mid subfield | −6.320 | (9.478) | −6.561 | (16.430) | 0.965 |
| RPE-BM foveal center | −3.740 | (26.562) | −12.512 | (33.585) | 0.486 |
| RPE-BM central subfield | −0.118 | (9.162) | −8.238 | (30.774) | 0.397 |
| RPE-BM mid subfield | 1.114 | (4.446) | −5.287 | (17.303) | 0.237 |
| ELM-RPE foveal center | −12.189 | (38.267) | 0.000 | (17.833) | 0.333 |
| ELM-RPE central subfield | −2.722 | (6.276) | −3.102 | (3.866) | 0.860 |
| ELM-RPE mid subfield | −1.044 | (5.688) | −2.141 | (4.001) | 0.591 |
| Inner retina central subfield | 1.896 | (15.489) | 5.864 | (9.780) | 0.462 |
| Inner retina mid subfield | 2.274 | (9.891) | 5.512 | (12.749) | 0.495 |
| ELM-EZ central subfield | −1.332 | (8.638) | −2.322 | (3.909) | 0.722 |
| ELM-EZ mid subfield | 0.754 | (6.292) | −0.967 | (2.744) | 0.399 |
| ONL-EZ central subfield | −6.486 | (14.913) | −5.775 | (15.038) | 0.908 |
| ONL-EZ mid subfield | −4.522 | (10.111) | −5.386 | (15.685) | 0.874 |
| Change in volume, mm³ | | | | | |
| Total retinal | 0.091 | (0.448) | −0.188 | (0.406) | 0.125 |
| Total retinal central subfield | −0.004 | (0.009) | 0.000 | (0.009) | 0.255 |
| Total retinal mid subfield | −0.012 | (0.017) | −0.004 | (0.020) | 0.266 |
| EZ-RPE | 0.005 | (0.136) | −0.059 | (0.176) | 0.331 |
| EZ-RPE central subfield | −0.001 | (0.005) | −0.001 | (0.003) | 0.797 |
| EZ-RPE mid subfield | −0.006 | (0.012) | −0.004 | (0.012) | 0.712 |
| ONL-RPE | −0.026 | (0.223) | 0.009 | (0.547) | 0.837 |
| ONL-RPE central subfield | −0.006 | (0.011) | −0.005 | (0.013) | 0.849 |
| ONL-RPE mid subfield | −0.020 | (0.030) | −0.021 | (0.052) | 0.948 |
| RPE-BM | 0.037 | (0.072) | −0.049 | (0.110) | 0.034 |
| RPE-BM central subfield | 0.000 | (0.007) | −0.007 | (0.024) | 0.393 |
| RPE-BM mid subfield | 0.004 | (0.014) | −0.017 | (0.055) | 0.236 |
| ELM-RPE | 0.009 | (0.184) | 0.086 | (0.516) | 0.637 |
| ELM-RPE central subfield | −0.002 | (0.005) | −0.002 | (0.003) | 0.832 |
| ELM-RPE mid subfield | −0.003 | (0.018) | −0.007 | (0.013) | 0.582 |
| ELM-EZ central subfield | −0.001 | (0.007) | −0.002 | (0.003) | 0.730 |
| ELM-EZ mid subfield | 0.002 | (0.020) | −0.003 | (0.009) | 0.398 |
| ONL-EZ central subfield | −0.005 | (0.012) | −0.004 | (0.012) | 0.912 |
| ONL-EZ mid subfield | −0.014 | (0.032) | −0.017 | (0.049) | 0.861 |
| Map coverage, % | | | | | |
| 250 μm RPE-BM | 0.000 | (0.000) | −0.011 | (0.040) | 0.339 |
| 150 μm RPE-BM | 2.143 | (4.131) | 3.335 | (3.091) | 0.433 |
| 50 μm RPE-BM | −1.794 | (3.274) | −3.49 | (3.545) | 0.235 |
| 0 μm RPE-BM | 1.465 | (3.264) | 1.099 | (2.468) | 0.760 |
| 20 μm EZ | 1.288 | (1.754) | 3.574 | (9.082) | 0.409 |
| 10 μm EZ | 1.332 | (2.027) | 3.699 | (10.517) | 0.459 |
| 0 μm EZ | 1.469 | (2.374) | 3.679 | (10.682) | 0.497 |

Abbreviations: ELM-EZ, external limiting membrane-ellipsoid zone; ELM-RPE, external limiting membrane-retinal pigment epithelium; EZ, ellipsoid zone; EZ-RPE, ellipsoid zone-retinal pigment epithelium; ONL-EZ, outer nuclear layer-ellipsoid zone; ONL-RPE, outer nuclear layer-retinal pigment epithelium; RPE-BM, retinal pigment epithelium-Bruch's membrane.
One subject in the risuteganib nonresponder group was excluded because of a missing endpoint image.

No anatomical measurements showed a significant difference in the change from baseline at Week 32 between risuteganib super-responder eyes and nonresponder eyes, as summarized in Table 22, below:

TABLE 22
Quantitative Anatomical Measurements Change From Baseline at Week
32 for Risuteganib Nonresponder Eyes Versus Super-Risuteganib Eyes

| | Risuteganib | | Risuteganib | | Two-Sample |
|---|---|---|---|---|---|
| Measurement | Nonresponder | | Super-Responder | | T-test |
| Sector | n = 12 | | n = 8 | | P-value |
| Change in mean (SD) thickness, μm | | | | | |
| Total retinal foveal center | −9.112 | (37.435) | −7.569 | (36.457) | 0.928 |
| Total retinal central subfield | −5.981 | (10.604) | −2.202 | (12.024) | 0.483 |
| Total retinal mid subfield | −4.046 | (5.084) | −1.490 | (6.023) | 0.341 |
| EZ-RPE foveal center | −1.789 | (30.522) | 6.825 | (22.233) | 0.475 |
| EZ-RPE central subfield | −1.390 | (6.069) | −1.803 | (3.251) | 0.846 |
| EZ-RPE mid subfield | −1.798 | (3.956) | −1.445 | (4.583) | 0.861 |
| ONL-RPE foveal center | −7.626 | (40.364) | −6.825 | (33.142) | 0.962 |
| ONL-RPE central subfield | −7.877 | (14.446) | −10.961 | (17.173) | 0.682 |
| ONL-RPE mid subfield | −6.320 | (9.478) | −10.001 | (18.827) | 0.621 |
| RPE-BM foveal center | −3.740 | (26.562) | −14.381 | (39.152) | 0.515 |
| RPE-BM central subfield | −0.118 | (9.162) | −11.226 | (38.084) | 0.443 |
| RPE-BM mid subfield | 1.114 | (4.446) | −6.715 | (21.395) | 0.340 |
| ELM-RPE foveal center | −12.189 | (38.267) | 2.925 | (21.234) | 0.273 |
| ELM-RPE central subfield | −2.722 | (6.276) | −3.461 | (4.056) | 0.753 |
| ELM-RPE mid subfield | −1.044 | (5.688) | −2.493 | (4.363) | 0.528 |
| Inner retina central subfield | 1.896 | (15.489) | 8.759 | (10.923) | 0.261 |
| Inner retina mid subfield | 2.274 | (9.891) | 8.511 | (14.853) | 0.319 |
| ELM-EZ central subfield | −1.332 | (8.638) | −1.658 | (2.715) | 0.905 |
| ELM-EZ mid subfield | 0.754 | (6.292) | −1.049 | (2.460) | 0.385 |
| ONL-EZ central subfield | −6.486 | (14.913) | −9.158 | (16.526) | 0.718 |
| ONL-EZ mid subfield | −4.522 | (10.111) | −8.556 | (17.980) | 0.577 |
| Change in volume, mm3 | | | | | |
| Total retinal | 0.091 | (0.448) | −0.247 | (0.496) | 0.144 |
| Total retinal central subfield | −0.004 | (0.009) | −0.001 | (0.010) | 0.511 |
| Total retinal mid subfield | −0.012 | (0.017) | −0.005 | (0.020) | 0.436 |
| EZ-RPE | 0.005 | (0.136) | −0.051 | (0.190) | 0.481 |
| EZ-RPE central subfield | −0.001 | (0.005) | −0.001 | (0.003) | 0.801 |
| EZ-RPE mid subfield | −0.006 | (0.012) | −0.005 | (0.014) | 0.877 |
| ONL-RPE | −0.026 | (0.223) | 0.044 | (0.680) | 0.784 |
| ONL-RPE central subfield | −0.006 | (0.011) | −0.008 | (0.014) | 0.669 |
| ONL-RPE mid subfield | −0.020 | (0.030) | −0.032 | (0.059) | 0.606 |
| RPE-BM | 0.037 | (0.072) | −0.048 | (0.115) | 0.091 |
| RPE-BM central subfield | 0.000 | (0.007) | −0.009 | (0.030) | 0.440 |
| RPE-BM mid subfield | 0.004 | (0.014) | −0.021 | (0.068) | 0.337 |
| ELM-RPE | 0.009 | (0.184) | 0.171 | (0.625) | 0.497 |
| ELM-RPE central subfield | −0.002 | (0.005) | −0.003 | (0.003) | 0.718 |
| ELM-RPE mid subfield | −0.003 | (0.018) | −0.008 | (0.014) | 0.520 |
| ELM-EZ central subfield | −0.001 | (0.007) | −0.001 | (0.002) | 0.907 |
| ELM-EZ mid subfield | 0.002 | (0.020) | −0.003 | (0.008) | 0.384 |
| ONL-EZ central subfield | −0.005 | (0.012) | −0.007 | (0.013) | 0.716 |
| ONL-EZ mid subfield | −0.014 | (0.032) | −0.027 | (0.056) | 0.565 |
| Map coverage, % | | | | | |
| 250 μm RPE-BM | 0.000 | (0.000) | −0.017 | (0.048) | 0.351 |
| 150 μm RPE-BM | 2.143 | (4.131) | 2.943 | (3.234) | 0.634 |
| 50 μm RPE-BM | −1.794 | (3.274) | −3.222 | (4.095) | 0.424 |
| 0 μm RPE-BM | 1.465 | (3.264) | 1.546 | (2.974) | 0.955 |
| 20 μm EZ | 1.288 | (1.754) | 4.506 | (11.065) | 0.441 |
| 10 μm EZ | 1.332 | (2.027) | 5.037 | (12.899) | 0.446 |
| 0 μm EZ | 1.469 | (2.374) | 5.026 | (13.116) | 0.472 |

Abbreviations: ELM-EZ, external limiting membrane-ellipsoid zone; ELM-RPE, external limiting membrane-retinal pigment epithelium; EZ, ellipsoid zone; EZ-RPE, ellipsoid zone-retinal pigment epithelium; ONL-EZ, outer nuclear layer-ellipsoid zone; ONL-RPE, outer nuclear layer-retinal pigment epithelium; RPE-BM, retinal pigment epithelium-Bruch's membrane.
NOTE:
One subject in the risuteganib nonresponder group was excluded because of a missing endpoint image.

Change in Anatomical Measurements Over Time of Risuteganib Subgroups vs Sham Arm. Sham eyes had significantly greater change in mean thickness from baseline at Week 12 in 3 different retinal metrics compared with the change in risuteganib nonresponder eyes from baseline at Week 32: mean total retinal central subfield thickness (1.659 vs −5.981 μm; P=0.043), mean total retinal mid subfield thickness (1.281 vs −4.046 μm; P=0.016), and mean ONL-RPE mid subfield thickness (0.778 vs −6.320 μm; P=0.047). This is summarized in Table 23 below.

TABLE 23
Quantitative Anatomical Measurements Change From Baseline at Week 32 for Risuteganib
Nonresponder Eyes Versus Change From Baseline at Week 12 for Sham Eyes

| | Risuteganib | | | | Two-Sample |
|---|---|---|---|---|---|
| Measurement | Nonresponder | | Sham | | T-test |
| Sector | n = 12 | | n = 13 | | P-value |
| Change in mean (SD) thickness, μm | | | | | |
| Total retinal foveal center | −9.112 | (37.435) | 1.045 | (28.248) | 0.455 |
| Total retinal central subfield | −5.981 | (10.604) | 1.659 | (6.169) | 0.043 |
| Total retinal mid subfield | −4.046 | (5.084) | 1.281 | (5.140) | 0.016 |
| EZ-RPE foveal center | −1.789 | (30.522) | −3.900 | (15.437) | 0.832 |
| EZ-RPE central subfield | −1.390 | (6.069) | 0.439 | (5.330) | 0.433 |
| EZ-RPE mid subfield | −1.798 | (3.956) | 0.412 | (4.151) | 0.186 |
| ONL-RPE foveal center | −7.626 | (40.364) | −11.267 | (33.575) | 0.809 |
| ONL-RPE central subfield | −7.877 | (14.446) | −1.441 | (8.454) | 0.196 |
| ONL-RPE mid subfield | −6.320 | (9.478) | 0.778 | (7.014) | 0.047 |
| RPE-BM foveal center | −3.740 | (26.562) | −1.643 | (17.883) | 0.821 |
| RPE-BM central subfield | −0.118 | (9.162) | −4.036 | (9.785) | 0.312 |
| RPE-BM mid subfield | 1.114 | (4.446) | −3.150 | (7.728) | 0.104 |
| ELM-RPE foveal center | −12.189 | (38.267) | −7.200 | (26.824) | 0.712 |
| ELM-RPE central subfield | −2.722 | (6.276) | −1.959 | (9.803) | 0.817 |
| ELM-RPE mid subfield | −1.044 | (5.688) | −1.720 | (6.481) | 0.784 |
| Inner retina central subfield | 1.896 | (15.489) | 3.100 | (7.421) | 0.810 |
| Inner retina mid subfield | 2.274 | (9.891) | 0.503 | (6.902) | 0.612 |
| ELM-EZ central subfield | −1.332 | (8.638) | −2.398 | (6.175) | 0.728 |
| ELM-EZ mid subfield | 0.754 | (6.292) | −2.132 | (5.105) | 0.224 |
| ONL-EZ central subfield | −6.486 | (14.913) | −1.880 | (8.602) | 0.362 |
| ONL-EZ mid subfield | −4.522 | (10.111) | 0.365 | (6.790) | 0.176 |
| Change in volume, mm³ | | | | | |
| Total retinal | 0.091 | (0.448) | −0.464 | (0.709) | 0.028 |
| Total retinal central subfield | −0.004 | (0.009) | 0.002 | (0.006) | 0.047 |
| Total retinal mid subfield | −0.012 | (0.017) | 0.005 | (0.017) | 0.020 |
| EZ-RPE | 0.005 | (0.136) | −0.043 | (0.112) | 0.347 |
| EZ-RPE central subfield | −0.001 | (0.005) | 0.000 | (0.004) | 0.432 |
| EZ-RPE mid subfield | −0.006 | (0.012) | 0.001 | (0.013) | 0.190 |
| ONL-RPE | −0.026 | (0.223) | −0.167 | (0.317) | 0.210 |
| ONL-RPE central subfield | −0.006 | (0.011) | −0.001 | (0.007) | 0.192 |
| ONL-RPE mid subfield | −0.020 | (0.030) | 0.003 | (0.022) | 0.046 |
| RPE-BM | 0.037 | (0.072) | −0.071 | (0.091) | 0.003 |
| RPE-BM central subfield | 0.000 | (0.007) | −0.003 | (0.008) | 0.307 |
| RPE-BM mid subfield | 0.004 | (0.014) | −0.010 | (0.024) | 0.103 |
| ELM-RPE | 0.009 | (0.184) | −0.103 | (0.369) | 0.344 |
| ELM-RPE central subfield | −0.002 | (0.005) | −0.001 | (0.008) | 0.827 |
| ELM-RPE mid subfield | −0.003 | (0.018) | −0.005 | (0.021) | 0.784 |
| ELM-EZ central subfield | −0.001 | (0.007) | −0.002 | (0.005) | 0.724 |
| ELM-EZ mid subfield | 0.002 | (0.020) | −0.007 | (0.016) | 0.224 |
| ONL-EZ central subfield | −0.005 | (0.012) | −0.001 | (0.007) | 0.355 |
| ONL-EZ mid subfield | −0.014 | (0.032) | 0.001 | (0.021) | 0.172 |
| Map coverage, % | | | | | |
| 250 μm RPE-BM | 0.000 | (0.000) | 0.058 | (0.167) | 0.236 |
| 150 μm RPE-BM | 2.143 | (4.131) | 3.376 | (5.205) | 0.517 |
| 50 μm RPE-BM | −1.794 | (3.274) | −3.674 | (5.057) | 0.279 |
| 0 μm RPE-BM | 1.465 | (3.264) | −0.144 | (0.828) | 0.122 |
| 20 μm EZ | 1.288 | (1.754) | 0.444 | (1.592) | 0.222 |
| 10 μm EZ | 1.332 | (2.027) | 0.476 | (1.226) | 0.222 |
| 0 μm EZ | 1.469 | (2.374) | 0.530 | (1.302) | 0.242 |

Abbreviations: ELM-EZ, external limiting membrane-ellipsoid zone; ELM-RPE, external limiting membrane-retinal pigment epithelium; EZ, ellipsoid zone; EZ-RPE, ellipsoid zone-retinal pigment epithelium; ONL-EZ, outer nuclear layer-ellipsoid zone; ONL-RPE, outer nuclear layer-retinal pigment epithelium; RPE-BM, retinal pigment epithelium-Bruch's membrane.
NOTE:
One subject in the risuteganib nonresponder group and one subject in the sham group were excluded because of a missing endpoint image.

The same metrics in sham eyes also had significantly greater change in volume from baseline at Week 12 compared with the change in risuteganib non-responder eyes from baseline at Week 32: total retinal central subfield volume (0.002 vs −0.004 mm³; P=0.047), total retinal mid subfield volume (0.005 vs −0.012 mm³; P=0.020), and ONL-RPE mid subfield volume (0.003 vs −0.020 mm³; P=0.046). In addition, the changes from baseline at Week 12 in sham eyes in total retinal volume (−0.464 vs 0.091 mm³; P=0.028) and RPE-BM volume (−0.071 vs 0.037 mm³; P=0.003) were significantly smaller compared with the changes from baseline at Week 32 in non-responder eyes.

No other anatomical measurements showed a significant difference in the change from baseline at Week 32 between risuteganib non-responder eyes and sham eyes.

No anatomical measurements showed a significant difference between the change from baseline at Week 32 in risuteganib responder eyes and the change from baseline at Week 12 in sham eyes, as summarized in Table 24, below:

TABLE 24
Quantitative Anatomical Measurements Change From Baseline at Week 32 for Risuteganib
Responder Eyes Versus Change From Baseline at Week 12 for Sham Eyes

| | Risuteganib | | | | Two-Sample |
|---|---|---|---|---|---|
| Measurement | Responder | | Sham | | T-test |
| Sector | n = 12 | | n = 13 | | P-value |
| Change in mean (SD) thickness, μm | | | | | |
| Total retinal foveal center | 0.804 | (32.231) | 1.045 | (28.248) | 0.984 |
| Total retinal central subfield | −0.691 | (10.370) | 1.659 | (6.169) | 0.504 |
| Total retinal mid subfield | −1.049 | (6.183) | 1.281 | (5.140) | 0.319 |
| EZ-RPE foveal center | 0.975 | (21.174) | −3.900 | (15.437) | 0.521 |
| EZ-RPE central subfield | −0.779 | (3.229) | 0.439 | (5.330) | 0.494 |
| EZ-RPE mid subfield | −1.174 | (3.772) | 0.412 | (4.151) | 0.327 |
| ONL-RPE foveal center | 0.650 | (30.970) | −11.267 | (33.575) | 0.365 |
| ONL-RPE central subfield | −6.555 | (15.778) | −1.441 | (8.454) | 0.333 |
| ONL-RPE mid subfield | −6.561 | (16.430) | 0.778 | (7.014) | 0.173 |
| RPE-BM foveal center | −12.512 | (33.585) | −1.643 | (17.883) | 0.333 |
| RPE-BM central subfield | −8.238 | (30.774) | −4.036 | (9.785) | 0.658 |
| RPE-BM mid subfield | −5.287 | (17.303) | −3.150 | (7.728) | 0.700 |
| ELM-RPE foveal center | −0.000 | (17.833) | −7.200 | (26.824) | 0.435 |
| ELM-RPE central subfield | −3.102 | (3.866) | −1.959 | (9.803) | 0.702 |
| ELM-RPE mid subfield | −2.141 | (4.001) | −1.720 | (6.481) | 0.846 |
| Inner retina central subfield | 5.864 | (9.780) | 3.100 | (7.421) | 0.438 |
| Inner retina mid subfield | 5.512 | (12.749) | 0.503 | (6.902) | 0.244 |
| ELM-EZ central subfield | −2.322 | (3.909) | −2.398 | (6.175) | 0.971 |
| ELM-EZ mid subfield | −0.967 | (2.744) | −2.132 | (5.105) | 0.481 |
| ONL-EZ central subfield | −5.775 | (15.038) | −1.880 | (8.602) | 0.442 |
| ONL-EZ mid subfield | −5.386 | (15.685) | 0.365 | (6.790) | 0.259 |
| Change in volume, mm³ | | | | | |
| Total retinal | −0.188 | (0.406) | −0.464 | (0.709) | 0.241 |
| Total retinal central subfield | 0.000 | (0.009) | 0.002 | (0.006) | 0.462 |
| Total retinal mid subfield | −0.004 | (0.020) | 0.005 | (0.017) | 0.287 |
| EZ-RPE | −0.059 | (0.176) | −0.043 | (0.112) | 0.795 |
| EZ-RPE central subfield | −0.001 | (0.003) | 0.000 | (0.004) | 0.463 |
| EZ-RPE mid subfield | −0.004 | (0.012) | 0.001 | (0.013) | 0.322 |
| ONL-RPE | 0.009 | (0.547) | −0.167 | (0.317) | 0.342 |
| ONL-RPE central subfield | −0.005 | (0.013) | −0.001 | (0.007) | 0.316 |
| ONL-RPE mid subfield | −0.021 | (0.052) | 0.003 | (0.022) | 0.166 |
| RPE-BM | −0.049 | (0.110) | −0.071 | (0.091) | 0.601 |
| RPE-BM central subfield | −0.007 | (0.024) | −0.003 | (0.008) | 0.654 |
| RPE-BM mid subfield | −0.017 | (0.055) | −0.010 | (0.024) | 0.696 |
| ELM-RPE | 0.086 | (0.516) | −0.103 | (0.369) | 0.310 |
| ELM-RPE central subfield | −0.002 | (0.003) | −0.001 | (0.008) | 0.689 |
| ELM-RPE mid subfield | −0.007 | (0.013) | −0.005 | (0.021) | 0.835 |
| ELM-EZ central subfield | −0.002 | (0.003) | −0.002 | (0.005) | 0.954 |
| ELM-EZ mid subfield | −0.003 | (0.009) | −0.007 | (0.016) | 0.484 |
| ONL-EZ central subfield | −0.004 | (0.012) | −0.001 | (0.007) | 0.426 |
| ONL-EZ mid subfield | −0.017 | (0.049) | 0.001 | (0.021) | 0.250 |
| Map coverage, % | | | | | |
| 250 μm RPE-BM | −0.011 | (0.040) | 0.058 | (0.167) | 0.170 |
| 150 μm RPE-BM | 3.335 | (3.091) | 3.376 | (5.205) | 0.981 |
| 50 μm RPE-BM | −3.494 | (3.545) | −3.674 | (5.057) | 0.918 |
| 0 μm RPE-BM | 1.099 | (2.468) | −0.144 | (0.828) | 0.120 |
| 20 μm EZ | 3.574 | (9.082) | 0.444 | (1.592) | 0.263 |
| 10 μm EZ | 3.699 | (10.517) | 0.476 | (1.226) | 0.314 |
| 0 μm EZ | 3.679 | (10.682) | 0.530 | (1.302) | 0.332 |

Abbreviations: ELM-EZ, external limiting membrane-ellipsoid zone; ELM-RPE, external limiting membrane-retinal pigment epithelium; EZ, ellipsoid zone; EZ-RPE, ellipsoid zone-retinal pigment epithelium; ONL-EZ, outer nuclear layer-ellipsoid zone; ONL-RPE, outer nuclear layer-retinal pigment epithelium; RPE-BM, retinal pigment epithelium-Bruch's membrane.
NOTE:
One subject in the sham group was excluded because of a missing endpoint image.

Change in Anatomical Measurements Over Time by Treatment Arm. Eyes treated with sham had statistically significantly greater change in mean thickness from baseline at Week 12 compared with the change from baseline at Week 32 for eyes that were treated with risuteganib in mean total retinal mid subfield thickness (1.281 vs −2.548 μm; P=0.048) and mean ONL-RPE mid subfield thickness (0.778 vs −6.441 μm; P=0.036) This is summarized in Table 25, below.

TABLE 25
Quantitative Anatomical Measurements Change From Baseline at Week 32 for
Risuteganib Arm Versus Change From Baseline at Week 12 for Sham Arm

| | | | | | Two-Sample |
|---|---|---|---|---|---|
| Measurement | Risuteganib | | Sham | | T-test |
| Sector | n = 24 | | n = 13 | | P-value |
| Change in mean (SD) thickness, μm | | | | | |
| Total retinal foveal center | −4.154 | (34.536) | 1.045 | (28.248) | 0.625 |
| Total retinal central subfield | −3.336 | (10.607) | 1.659 | (6.169) | 0.079 |
| Total retinal mid subfield | −2.548 | (5.743) | 1.281 | (5.140) | 0.048 |
| EZ-RPE foveal center | −0.407 | (25.729) | −3.900 | (15.437) | 0.609 |
| EZ-RPE central subfield | −1.085 | (4.765) | 0.439 | (5.330) | 0.398 |
| EZ-RPE mid subfield | −1.486 | (3.793) | 0.412 | (4.151) | 0.184 |
| ONL-RPE foveal center | −3.488 | (35.437) | −11.267 | (33.575) | 0.515 |
| ONL-RPE central subfield | −7.216 | (14.809) | −1.441 | (8.454) | 0.140 |
| ONL-RPE mid subfield | −6.441 | (13.118) | 0.778 | (7.014) | 0.036 |
| RPE-BM foveal center | −8.126 | (29.949) | −1.643 | (17.883) | 0.416 |
| RPE-BM central subfield | −4.178 | (22.590) | −4.036 | (9.785) | 0.979 |
| RPE-BM mid subfield | −2.086 | (12.780) | −3.150 | (7.728) | 0.755 |
| ELM-RPE foveal center | −6.094 | (29.853) | −7.200 | (26.824) | 0.909 |
| ELM-RPE central subfield | −2.912 | (5.101) | −1.959 | (9.803) | 0.748 |
| ELM-RPE mid subfield | −1.593 | (4.841) | −1.720 | (6.481) | 0.951 |
| Inner retina central subfield | 3.880 | (12.829) | 3.100 | (7.421) | 0.816 |
| Inner retina mid subfield | 3.893 | (11.281) | 0.503 | (6.902) | 0.265 |
| ELM-EZ central subfield | −1.827 | (6.577) | −2.398 | (6.175) | 0.795 |
| ELM-EZ mid subfield | −0.106 | (4.828) | −2.132 | (5.105) | 0.252 |
| ONL-EZ central subfield | −6.131 | (14.651) | −1.880 | (8.602) | 0.274 |
| ONL-EZ mid subfield | −4.954 | (12.913) | 0.365 | (6.790) | 0.110 |
| Change in volume, mm³ | | | | | |
| Total retinal | −0.048 | (0.442) | −0.464 | (0.709) | 0.071 |
| Total retinal central subfield | −0.002 | (0.009) | 0.002 | (0.006) | 0.080 |
| Total retinal mid subfield | −0.008 | (0.019) | 0.005 | (0.017) | 0.049 |
| EZ-RPE | −0.027 | (0.157) | −0.043 | (0.112) | 0.718 |
| EZ-RPE central subfield | −0.001 | (0.004) | 0.000 | (0.004) | 0.385 |
| EZ-RPE mid subfield | −0.005 | (0.012) | 0.001 | (0.013) | 0.186 |
| ONL-RPE | −0.009 | (0.409) | −0.167 | (0.317) | 0.200 |
| ONL-RPE central subfield | −0.005 | (0.012) | −0.001 | (0.007) | 0.133 |
| ONL-RPE mid subfield | −0.020 | (0.041) | 0.003 | (0.022) | 0.033 |
| RPE-BM | −0.006 | (0.101) | −0.071 | (0.091) | 0.058 |
| RPE-BM central subfield | −0.003 | (0.018) | −0.003 | (0.008) | 0.977 |
| RPE-BM mid subfield | −0.007 | (0.040) | −0.010 | (0.024) | 0.757 |
| ELM-RPE | 0.048 | (0.381) | −0.103 | (0.369) | 0.253 |
| ELM-RPE central subfield | −0.002 | (0.004) | −0.001 | (0.008) | 0.745 |
| ELM-RPE mid subfield | −0.005 | (0.015) | −0.005 | (0.021) | 0.957 |
| ELM-EZ central subfield | −0.001 | (0.005) | −0.002 | (0.005) | 0.783 |
| ELM-EZ mid subfield | 0.000 | (0.015) | −0.007 | (0.016) | 0.252 |
| ONL-EZ central subfield | −0.005 | (0.012) | −0.001 | (0.007) | 0.262 |
| ONL-EZ mid subfield | −0.016 | (0.041) | 0.001 | (0.021) | 0.103 |
| Map coverage, % | | | | | |
| 250 μm RPE-BM | −0.006 | (0.028) | 0.058 | (0.167) | 0.198 |
| 150 μm RPE-BM | 2.739 | (3.620) | 3.376 | (5.205) | 0.699 |
| 50 μm RPE-BM | −2.644 | (3.448) | −3.674 | (5.057) | 0.520 |
| 0 μm RPE-BM | 1.282 | (2.836) | −0.144 | (0.828) | 0.029 |
| 20 μm EZ | 2.431 | (6.502) | 0.444 | (1.592) | 0.167 |
| 10 μm EZ | 2.515 | (7.505) | 0.476 | (1.226) | 0.205 |
| 0 μm EZ | 2.574 | (7.651) | 0.530 | (1.302) | 0.214 |

Abbreviations: ELM-EZ, external limiting membrane-ellipsoid zone; ELM-RPE, external limiting membrane-retinal pigment epithelium; EZ, ellipsoid zone; EZ-RPE, ellipsoid zone-retinal pigment epithelium; ONL-EZ, outer nuclear layer-ellipsoid zone; ONL-RPE, outer nuclear layer-retinal pigment epithelium; RPE-BM, retinal pigment epithelium-Bruch's membrane.
NOTE:
One subject in the risuteganib arm and one in the sham arm was excluded because of a missing endpoint image.

The same metrics in sham eyes also had significantly greater change in volume from baseline at Week 12 compared with the change from baseline at Week 32 for risuteganib eyes: total retinal mid subfield volume (0.005 vs −0.008 mm³; P=0.049) and ONL-RPE mid subfield volume (0.003 vs −0.020 mm³; P=0.033).

No other anatomical measurements showed a significant difference between the change from baseline at Week 32 in risuteganib eyes and the change from baseline at Week 12 in sham eyes.

Change in Anatomical Measurements Over Time within Risuteganib Responder Groups

Paired-eye analysis showed a significant decline in mean thickness from baseline at Week 32 in risuteganib nonresponder eyes in mean total retinal mid subfield thickness (−4.046 μm; P=0.019) and mean ONL-RPE mid subfield thickness (−6.320 μm; P=0.041) and in risuteganib responder and super-responder eyes in mean ELM-RPE central subfield thickness (−3.102 μm; P=0.018 and −3.461 μm; P=0.047, respectively, as summarized in Table 26, below.

TABLE 26
Quantitative Paired Anatomical Measurements at Baseline and at Week 32 for Risuteganib Responder Groups

| | Risuteganib | | Two-Sample | Risuteganib | | Two-Sample | Risuteganib | | Two-Sample |
|---|---|---|---|---|---|---|---|---|---|
| Measurement | Nonresponder | | T-test | Responder | | T-test | Super-Responder | | T-test |
| Sector | n = 12 | | P-value | n = 12 | | P-value | n = 8 | | P-value |
| Change in mean (SD) | | | | | | | | | |
| thickness, μm | | | | | | | | | |
| Total retinal foveal | −9.112 | (37.435) | 0.417 | 0.804 | (32.231) | 0.933 | −7.569 | (36.457) | 0.575 |
| center | | | | | | | | | |
| Total retinal central | −5.981 | (10.604) | 0.077 | −0.691 | (10.370) | 0.822 | −2.202 | (12.024) | 0.620 |
| subfield | | | | | | | | | |
| Total retinal mid subfield | −4.046 | (5.084) | 0.019 | −1.049 | (6.183) | 0.569 | −1.490 | (6.023) | 0.507 |
| EZ-RPE foveal center | −1.789 | (30.522) | 0.843 | 0.975 | (21.174) | 0.876 | 6.825 | (22.233) | 0.414 |
| EZ-RPE central subfield | −1.390 | (6.069) | 0.444 | −0.779 | (3.229) | 0.421 | −1.803 | (3.251) | 0.161 |
| EZ-RPE mid subfield | −1.798 | (3.956) | 0.144 | −1.174 | (3.772) | 0.304 | −1.445 | (4.583) | 0.402 |
| ONL-RPE foveal center | −7.626 | (40.364) | 0.526 | 0.650 | (30.970) | 0.943 | −6.825 | (33.142) | 0.579 |
| ONL-RPE central | −7.877 | (14.446) | 0.086 | −6.555 | (15.778) | 0.178 | −10.961 | (17.173) | 0.114 |
| subfield | | | | | | | | | |
| ONL-RPE mid subfield | −6.320 | (9.478) | 0.041 | −6.561 | (16.430) | 0.194 | −10.001 | (18.827) | 0.177 |
| RPE-BM foveal center | −3.740 | (26.562) | 0.635 | −12.512 | (33.585) | 0.223 | −14.381 | (39.152) | 0.333 |
| RPE-BM central subfield | −0.118 | (9.162) | 0.965 | −8.238 | (30.774) | 0.374 | −11.226 | (38.084) | 0.432 |
| RPE-BM mid subfield | 1.114 | (4.446) | 0.404 | −5.287 | (17.303) | 0.313 | −6.715 | (21.395) | 0.404 |
| ELM-RPE foveal center | −12.189 | (38.267) | 0.293 | 0.000 | (17.833) | 1.000 | 2.925 | (21.234) | 0.708 |
| ELM-RPE central | −2.722 | (6.276) | 0.161 | −3.102 | (3.866) | 0.018 | −3.461 | (4.056) | 0.047 |
| subfield | | | | | | | | | |
| ELM-RPE mid subfield | −1.044 | (5.688) | 0.538 | −2.141 | (4.001) | 0.091 | −2.493 | (4.363) | 0.150 |
| Inner retina central | 1.896 | (15.489) | 0.680 | 5.864 | (9.780) | 0.062 | 8.759 | (10.923) | 0.058 |
| subfield | | | | | | | | | |
| Inner retina mid subfield | 2.274 | (9.891) | 0.443 | 5.512 | (12.749) | 0.162 | 8.511 | (14.853) | 0.149 |
| ELM-EZ central subfield | −1.332 | (8.638) | 0.604 | −2.322 | (3.909) | 0.064 | −1.658 | (2.715) | 0.128 |
| ELM-EZ mid subfield | 0.754 | (6.292) | 0.686 | −0.967 | (2.744) | 0.248 | −1.049 | (2.460) | 0.267 |
| ONL-EZ central subfield | −6.486 | (14.913) | 0.160 | −5.775 | (15.038) | 0.210 | −9.158 | (16.526) | 0.161 |
| ONL-EZ mid subfield | −4.522 | (10.111) | 0.150 | −5.386 | (15.685) | 0.259 | −8.556 | (17.980) | 0.220 |
| Change in volume, mm³ | | | | | | | | | |
| Total retinal | 0.091 | (0.448) | 0.496 | −0.188 | (0.406) | 0.138 | −0.247 | (0.496) | 0.203 |
| Total retinal central | −0.004 | (0.009) | 0.119 | 0.000 | (0.009) | 0.952 | −0.001 | (0.010) | 0.700 |
| subfield | | | | | | | | | |
| Total retinal mid subfield | −0.012 | (0.017) | 0.027 | −0.004 | (0.020) | 0.560 | −0.005 | (0.020) | 0.498 |
| EZ-RPE | 0.005 | (0.136) | 0.898 | −0.059 | (0.176) | 0.271 | −0.051 | (0.190) | 0.468 |
| EZ-RPE central subfield | −0.001 | (0.005) | 0.493 | −0.001 | (0.003) | 0.460 | −0.001 | (0.003) | 0.171 |
| EZ-RPE mid subfield | −0.006 | (0.012) | 0.150 | −0.004 | (0.012) | 0.303 | −0.005 | (0.014) | 0.399 |
| ONL-RPE | −0.026 | (0.223) | 0.690 | 0.009 | (0.547) | 0.954 | 0.044 | (0.680) | 0.860 |
| ONL-RPE central | −0.006 | (0.011) | 0.105 | −0.005 | (0.013) | 0.202 | −0.008 | (0.014) | 0.123 |
| subfield | | | | | | | | | |
| ONL-RPE mid subfield | −0.020 | (0.030) | 0.044 | −0.021 | (0.052) | 0.192 | −0.032 | (0.059) | 0.174 |
| RPE-BM | 0.037 | (0.072) | 0.101 | −0.049 | (0.110) | 0.149 | −0.048 | (0.115) | 0.279 |
| RPE-BM central subfield | 0.000 | (0.007) | 0.996 | −0.007 | (0.024) | 0.377 | −0.009 | (0.030) | 0.434 |
| RPE-BM mid subfield | 0.004 | (0.014) | 0.397 | −0.017 | (0.055) | 0.312 | −0.021 | (0.068) | 0.403 |
| ELM-RPE | 0.009 | (0.184) | 0.864 | 0.086 | (0.516) | 0.577 | 0.171 | (0.625) | 0.464 |
| ELM-RPE central | −0.002 | (0.005) | 0.202 | −0.002 | (0.003) | 0.021 | −0.003 | (0.003) | 0.048 |
| subfield | | | | | | | | | |
| ELM-RPE mid subfield | −0.003 | (0.018) | 0.558 | −0.007 | (0.013) | 0.094 | −0.008 | (0.014) | 0.155 |
| ELM-EZ central subfield | −0.001 | (0.007) | 0.620 | −0.002 | (0.003) | 0.070 | −0.001 | (0.002) | 0.134 |
| ELM-EZ mid subfield | 0.002 | (0.020) | 0.683 | −0.003 | (0.009) | 0.252 | −0.003 | (0.008) | 0.275 |
| ONL-EZ central subfield | −0.005 | (0.012) | 0.183 | −0.004 | (0.012) | 0.231 | −0.007 | (0.013) | 0.171 |
| ONL-EZ mid subfield | −0.014 | (0.032) | 0.155 | −0.017 | (0.049) | 0.257 | −0.027 | (0.056) | 0.217 |
| Map coverage, % | | | | | | | | | |
| 250 μm RPE-BM | 0.000 | (0.000) | — | −0.011 | (0.040) | 0.339 | −0.017 | (0.048) | 0.351 |
| 150 μm RPE-BM | 2.143 | (4.131) | 0.100 | 3.335 | (3.091) | 0.003 | 2.943 | (3.234) | 0.037 |
| 50 μm RPE-BM | −1.794 | (3.274) | 0.084 | −3.494 | (3.545) | 0.006 | −3.222 | (4.095) | 0.061 |
| 0 μm RPE-BM | 1.465 | (3.264) | 0.148 | 1.099 | (2.468) | 0.151 | 1.546 | (2.974) | 0.185 |
| 20 μm EZ | 1.288 | (1.754) | 0.027 | 3.574 | (9.082) | 0.200 | 4.506 | (11.065) | 0.287 |
| 10 μm EZ | 1.332 | (2.027) | 0.044 | 3.699 | (10.517) | 0.249 | 5.037 | (12.899) | 0.306 |
| 0 μm EZ | 1.469 | (2.374) | 0.055 | 3.679 | (10.682) | 0.258 | 5.026 | (13.116) | 0.314 |

Abbreviations: ELM-EZ, external limiting membrane-ellipsoid zone; ELM-RPE, external limiting membrane-retinal pigment epithelium; EZ, ellipsoid zone; EZ-RPE, ellipsoid zone-retinal pigment epithelium; ONL-EZ, outer nuclear layer-ellipsoid zone; ONL-RPE, outer nuclear layer-retinal pigment epithelium; RPE-BM, retinal pigment epithelium-Bruch's membrane.
NOTE:
One subject in the risuteganib nonresponder group was excluded because of a missing endpoint image.

The same metrics also had a significant decline in volume from baseline at Week 32 in the same groups of eyes: total retinal mid subfield volume (−0.012 mm³; P=0.027) and ONL-RPE mid subfield volume (−0.020 mm³; P=0.044) in nonresponder eyes and ELM-RPE central subfield volume in responder and super-responder eyes (−0.002 mm³; P=0.021 and −0.003 mm³; P=0.048, respectively).

A significant difference in map coverage from baseline at Week 32 was observed in risuteganib nonresponder eyes in <20 μm EZ (+1.288%; P=0.027) and <10 μm EZ (+1.332%; P=0.044), in responder eyes in 150 μm RPE-BM (3.335%; P=0.003) and 50 μm RPE-BM (−3.494%; P=0.006), and in super-responder eyes in 150 μm RPE-BM (+2.943%; P=0.037).

No other anatomical measurements in any risuteganib responder group of eyes showed a significant difference from baseline at Week 32.

Change in Anatomical Measurements Over Time Within Treatment Arms. Paired-eye analysis showed a significant decline in mean thickness from baseline at Week 32 in the risuteganib arm in mean total retinal mid subfield thickness (−2.548 μm; P=0.040), mean ONL-RPE central subfield thickness (−7.216 μm; P=0.026), mean ONL-RPE mid subfield thickness (−6.441 μm; P=0.025), and mean ELM-RPE central subfield thickness (−2.912 μm; P=0.010). This is summarized in Table 27, below:

TABLE 27
Quantitative Paired Anatomical Measurements at Baseline and Week
32 for Risuteganib Arm and at Baseline and Week 12 for Sham Arm

| | | | Two-Sample | | | Two-Sample |
|---|---|---|---|---|---|---|
| Measurement | Risuteganib | | T-test | Sham | | T-test |
| Sector | n = 24 | | P-value | n = 13 | | P-value |
| Change in mean (SD) thickness, μm | | | | | | |
| Total retinal foveal center | −4.154 | (34.536) | 0.561 | 1.045 | (28.248) | 0.896 |
| Total retinal central subfield | −3.336 | (10.607) | 0.137 | 1.659 | (6.169) | 0.351 |
| Total retinal mid subfield | −2.548 | (5.743) | 0.040 | 1.281 | (5.140) | 0.387 |
| EZ-RPE foveal center | −0.407 | (25.729) | 0.939 | −3.900 | (15.437) | 0.380 |
| EZ-RPE central subfield | −1.085 | (4.765) | 0.276 | 0.439 | (5.330) | 0.771 |
| EZ-RPE mid subfield | −1.486 | (3.793) | 0.067 | 0.412 | (4.151) | 0.726 |
| ONL-RPE foveal center | −3.488 | (35.437) | 0.634 | −11.267 | (33.575) | 0.250 |
| ONL-RPE central subfield | −7.216 | (14.809) | 0.026 | −1.441 | (8.454) | 0.550 |
| ONL-RPE mid subfield | −6.441 | (13.118) | 0.025 | 0.778 | (7.014) | 0.696 |
| RPE-BM foveal center | −8.126 | (29.949) | 0.197 | −1.643 | (17.883) | 0.746 |
| RPE-BM central subfield | −4.178 | (22.590) | 0.374 | −4.036 | (9.785) | 0.163 |
| RPE-BM mid subfield | −2.086 | (12.780) | 0.432 | −3.150 | (7.728) | 0.167 |
| ELM-RPE foveal center | −6.094 | (29.853) | 0.328 | −7.200 | (26.824) | 0.352 |
| ELM-RPE central subfield | −2.912 | (5.101) | 0.010 | −1.959 | (9.803) | 0.485 |
| ELM-RPE mid subfield | −1.593 | (4.841) | 0.121 | −1.720 | (6.481) | 0.358 |
| Inner retina central subfield | 3.880 | (12.829) | 0.152 | 3.100 | (7.421) | 0.158 |
| Inner retina mid subfield | 3.893 | (11.281) | 0.104 | 0.503 | (6.902) | 0.797 |
| ELM-EZ central subfield | −1.827 | (6.577) | 0.187 | −2.398 | (6.175) | 0.187 |
| ELM-EZ mid subfield | −0.106 | (4.828) | 0.915 | −2.132 | (5.105) | 0.158 |
| ONL-EZ central subfield | −6.131 | (14.651) | 0.052 | −1.880 | (8.602) | 0.446 |
| ONL-EZ mid subfield | −4.954 | (12.913) | 0.073 | 0.365 | (6.790) | 0.849 |
| Change in volume, mm³ | | | | | | |
| Total retinal | −0.048 | (0.442) | 0.598 | −0.464 | (0.709) | 0.036 |
| Total retinal central subfield | −0.002 | (0.009) | 0.226 | 0.002 | (0.006) | 0.211 |
| Total retinal mid subfield | −0.008 | (0.019) | 0.051 | 0.005 | (0.017) | 0.341 |
| EZ-RPE | −0.027 | (0.157) | 0.412 | −0.043 | (0.112) | 0.192 |
| EZ-RPE central subfield | −0.001 | (0.004) | 0.324 | 0.000 | (0.004) | 0.694 |
| EZ-RPE mid subfield | −0.005 | (0.012) | 0.070 | 0.001 | (0.013) | 0.714 |
| ONL-RPE | −0.009 | (0.409) | 0.919 | −0.167 | (0.317) | 0.081 |
| ONL-RPE central subfield | −0.005 | (0.012) | 0.035 | −0.001 | (0.007) | 0.721 |
| ONL-RPE mid subfield | −0.020 | (0.041) | 0.025 | 0.003 | (0.022) | 0.656 |
| RPE-BM | −0.006 | (0.101) | 0.771 | −0.071 | (0.091) | 0.016 |
| RPE-BM central subfield | −0.003 | (0.018) | 0.385 | −0.003 | (0.008) | 0.168 |
| RPE-BM mid subfield | −0.007 | (0.040) | 0.433 | −0.010 | (0.024) | 0.168 |
| ELM-RPE | 0.048 | (0.381) | 0.547 | −0.103 | (0.369) | 0.335 |
| ELM-RPE central subfield | −0.002 | (0.004) | 0.016 | −0.001 | (0.008) | 0.521 |
| ELM-RPE mid subfield | −0.005 | (0.015) | 0.130 | −0.005 | (0.021) | 0.371 |
| ELM-EZ central subfield | −0.001 | (0.005) | 0.204 | −0.002 | (0.005) | 0.186 |
| ELM-EZ mid subfield | 0.000 | (0.015) | 0.919 | −0.007 | (0.016) | 0.158 |
| ONL-EZ central subfield | −0.005 | (0.012) | 0.064 | −0.001 | (0.007) | 0.554 |
| ONL-EZ mid subfield | −0.016 | (0.041) | 0.074 | 0.001 | (0.021) | 0.817 |
| Map coverage, % | | | | | | |
| 250 μm RPE-BM | −0.006 | (0.028) | 0.328 | 0.058 | (0.167) | 0.236 |
| 150 μm RPE-BM | 2.739 | (3.620) | 0.001 | 3.376 | (5.205) | 0.037 |
| 50 μm RPE-BM | −2.644 | (3.448) | 0.001 | −3.674 | (5.057) | 0.022 |
| 0 μm RPE-BM | 1.282 | (2.836) | 0.037 | −0.144 | (0.828) | 0.543 |
| 20 μm EZ | 2.431 | (6.502) | 0.080 | 0.444 | (1.592) | 0.334 |
| 10 μm EZ | 2.515 | (7.505) | 0.114 | 0.476 | (1.226) | 0.187 |
| 0 μm EZ | 2.574 | (7.651) | 0.113 | 0.530 | (1.302) | 0.168 |

Abbreviations: ELM-EZ, external limiting membrane-ellipsoid zone; ELM-RPE, external limiting membrane-retinal pigment epithelium; EZ, ellipsoid zone; EZ-RPE, ellipsoid zone-retinal pigment epithelium; ONL-EZ, outer nuclear layer-ellipsoid zone; ONL-RPE, outer nuclear layer-retinal pigment epithelium; RPE-BM, retinal pigment epithelium-Bruch's membrane.
NOTE:
One subject in the sham group was excluded because of a missing endpoint image.

A significant decline in volume from baseline at Week 32 was observed in the risuteganib arm in ONL-RPE central subfield volume (−0.005 mm³; P=0.035), ONL-RPE mid subfield volume (−0.020 mm³; P=0.025), and ELM-RPE mid subfield volume (−0.002 mm³; P=0.016), and in the sham arm from baseline at Week 12 in total retinal volume (−0.464 mm³; P=0.036) and RPE-BM volume (−0.071 mm³; P=0.016).

A significant difference in map coverage from baseline at Week 32 was observed in the risuteganib arm in 150 μm RPE-BM (2.739%; P=0.001), 50 μm RPE-BM (−2.644%; P=0.001), and 0 μm RPE-BM (1.282%; P=0.037), and from baseline at Week 12 in the sham arm in 150 μm RPE-BM (3.376%; P=0.037) and 50 μm RPE-BM (−3.674%; P=0.022).

Although these measurements are statistically significant, the absolute values of these changes are quite small and not clear if they are clinically meaningful. No other anatomical measurements showed a significant difference from baseline at Week 32 in the risuteganib arm or from baseline at Week 12 in the sham arm.

In this prospective, randomized, double-masked, US clinical trial, we have demonstrated a statistically significantly higher percentage of subjects that gained 8 letters or more after receiving 2 intravitreal injections with risuteganib compared with sham. This is the first time that a therapeutic agent has shown reversal of vision loss in dry AMD. Supporting assessments such as microperimetry and color vision show a trend of corroboration with the BCVA results, although they were not statistically significant.

A single injection of risuteganib demonstrated mild efficacy as seen in the 2 cohorts, subjects who received risuteganib at Week 0 and subjects in the sham group who crossed over and received risuteganib at Week 16. Two injections of risuteganib demonstrated an additive effect with further improvement in BCVA.

The peak effect of the drug is evident 12 weeks after treatment, with a mild decrease in therapeutic effect at 16 weeks. Repeat dosing demonstrated additive effect from the prior dose effect, peaking at 12 weeks and again with mild decrease in therapeutic effect after 16 weeks. These findings are similar to the 12-week peak effect observed with risuteganib in the Phase 2 DME studies.

Baseline retinal anatomy seems to be an important predictor of response. Subjects who had no GA in the central 6 mm and with intact external limiting membrane in the fovea consistently demonstrated significant improvement in vision with 2 risuteganib injections. Therefore, it is unknown if subjects with worse baseline anatomy would show improvement with more than 2 injections of Luminate. However, this subject population will be studied in future clinical studies.

The drug was well tolerated with no drug-related serious adverse events (SAEs). Floaters which recovered without sequelae were observed in some subjects.

### Suppression of Angiogenic and Inflammatory Gene Levels in OIR Mouse Retina

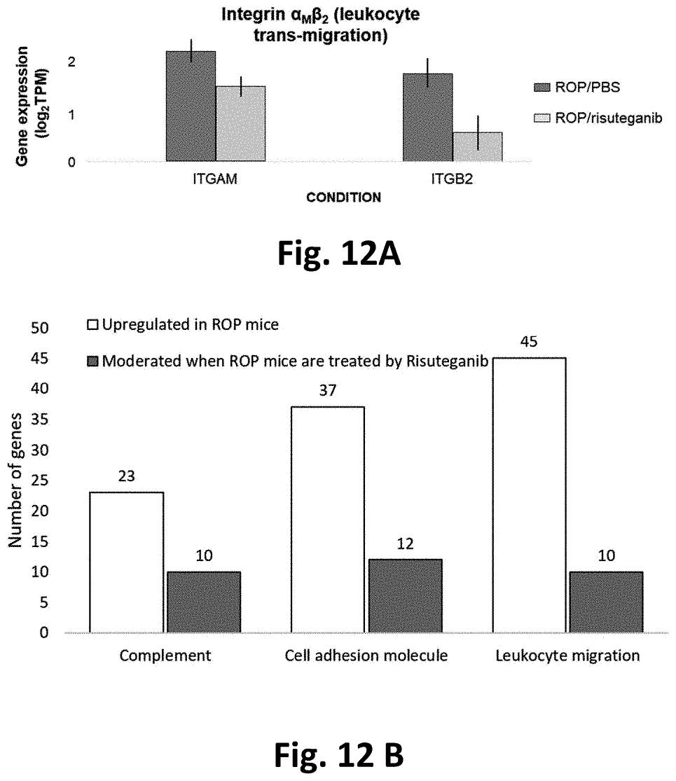

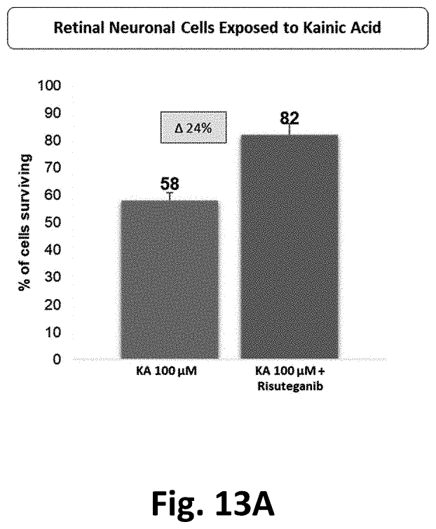

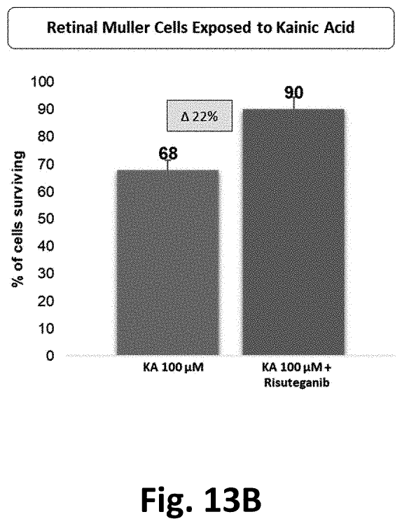

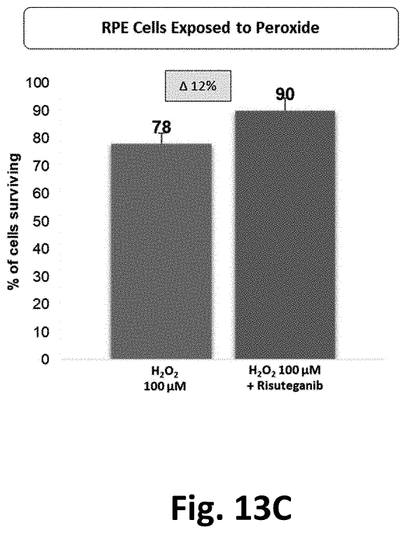

Purpose: This study used RNA-seq to identify the genes regulated in the mouse retina following risuteganib intravitreal injection. Analysis of the specific genes regulated by risuteganib enables identification of biological processes and pathways modulated by the oligopeptide. Results of this study are summarized in FIGS. 12A and 12B. This study indicates that anti-inflammatory effects of risuteganib are, at least in part, mediated by downregulation of integrin α_{M}β₂. Risuteganib causes reduced leukocyte attachment, reduced leukocyte trans-endothelial migration and reduced expression of complement 3 receptor.

Methods: OIR mouse pups received 5 days of hyperoxia (75% O₂) to obliterate developing retinal vessels. Following their return to room air, retinal neovascularization develops due to an imbalance in oxygen supply and demand. At the time of return to room air, both eyes of OIR pups received either vehicle injection or a single intravitreal injection of risuteganib solution at concentration of 10 μg/1 μL. A separate group of pups raised at room air served as control and received either vehicle or risuteganib solution injection consistent with the OIR mouse group. 5 days after injection, at the height of retinal neovascularization in OIR mice, all mice are sacrificed, retina tissue extracted for RNA isolation and RNA-seq. The generated reads were then aligned to the mouse reference genome/transcriptome and gene expression quantified for differential expression analysis and fold change calculation. The list of regulated genes was then submitted to identify biological processes and pathways that are regulated after risuteganib exposure compared to vehicle control in OIR mice or control mice, and in OIR retina compared to control retina that both received vehicle injections.

Results/Discussion: Risuteganib exposure regulated around 600 genes in the OIR retina with statistical significance, including 6 integrin subunits that are down regulated: α5, α6, αM, β1, β2, and β5. These integrins are involved in diverse set of biological functions including cell communication and adhesion during ischemia-activated angiogenesis and inflammation in the OIR retina. In particular, integrin αM and β2 subunits form the complement receptor 3 protein, which is expressed on leukocytes and functions in leukocytic adhesion, migration, and phagocytosis. Additionally, α5β1, α6β1, and αvβ5 integrins have all been implicated in regulating cell growth, survival and migration during angiogenesis.

When the entire list of regulated genes was considered, risuteganib appeared to have a general effect in moderating hypoxia-activated gene expression in angiogenesis and inflammation-related pathways. Among 11 biological pathways down-regulated by risuteganib, 10 are found to be up-regulated in the OIR retina. Many of these pathways are associated with angiogenesis and inflammation, such as PI3K-Akt signaling pathway and ECM-receptor interaction. In addition, several immune relevant pathways are suppressed by risuteganib, including complement and coagulation cascades and leukocyte transendothelial migration pathways. Importantly, when the specific regulated genes are considered, it was notable that many of the same genes activated in the OIR retina are suppressed by risuteganib. Overall, this unbiased transcriptome analysis suggest risuteganib solution injection was able to moderate many of the genes and biological pathways activated in the OIR retina, where ischemia generated an angiogenic and inflammatory condition that resembles retinal diseases such as DR and AMD.

Conclusion: Unbiased transcriptome analysis shows risuteganib solution injection moderated hypoxia-activated angiogenesis and inflammation-related gene expression.

### Neuroprotective Effects in Primary Mouse Müller Cells

Purpose: Investigate neuroprotective properties of risuteganib in primary mouse Müller cells exposed to kainic acid, a neuroexcitatory compound that activates glutamate receptors, resulting in overstimulation and cell death. Retinal Müller cells support normal functions of neurons and their dysregulation can leads to loss of homeostasis and neuronal cell death. Results of this study are summarized in Figures

Methods: Fresh retina were collected from CD1 mice and then mechanically dissociated with sterile Pasteur pipette into small aggregates and seeded into 35 mm culture dishes. All cultures were first left unchanged for 5-6 days and then replenished every 3-4 days. When the cell growth had reached around 80% confluency, retinal aggregates and debris were removed by media washes to form a purified cell monolayer. Cells were then exposed to the experimental conditions: (1) untreated control, (2) 1.0 mg/mL risuteganib, (3) 500 μM kainic acid (KA), and (4) 1.0 mg/mL risuteganib for 24 hours before 500 μM kainic acid exposure. 48 hours after kainic acid treatment, dead and live cell numbers were measured using Trypan blue exclusion assay on a hemocytometer.

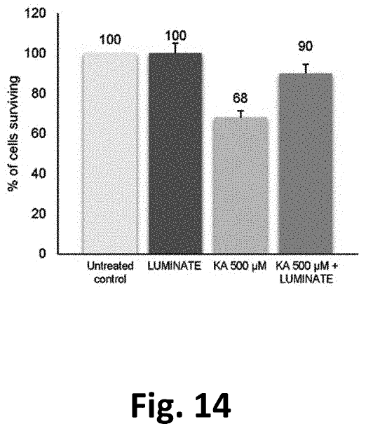

Results/Discussion: Risuteganib treatment alone did not induce detectable change in cell viability. As shown graphically in FIG. 14, kainic acid treatment alone reduced Muller cell viability by 32%, thereby establishing its toxicity to Müller cells, but risuteganib pre-treatment demonstrated protective effect by reducing the loss of Muller cell viability from 32% to 10%.

Conclusion: risuteganib alone did not alter cell viability, while pre-treatment demonstrated measurable protection against kainic acid-based cytotoxicity in primary mouse Müller cells.

### Neuroprotective Effects in Primary Mouse Retinal Neuron Cells

Purpose: Investigate neuroprotective properties of risuteganib in primary mouse retinal neuron cells exposed to kainic acid, a neuroexcitatory compound that activates glutamate receptors, resulting in overstimulation and cell death.

Methods: Fresh retina were collected from CD1 mice and then mechanically dissociated with sterile Pasteur pipette. Cell suspensions were then dispensed into petri dish and incubated for 6 hours. Cells were then exposed to the experimental conditions: (1) untreated control, (2) 1.0 mg/mL risuteganib, (3) 500 μM kainic acid (KA), and (4) 1.0 mg/mL risuteganib for 24 hours before 500 μM kainic acid exposure. 8 hours after kainic acid treatment, dead and live cell numbers were measured using Trypan blue exclusion assay on a hemocytometer.

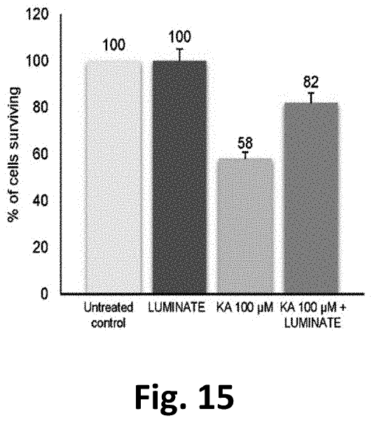

Results/Discussion: As shown graphically in FIG. 15, Risuteganib (Luminate) treatment alone did not induce detectable change in cell viability. However, treatment with kainic acid alone reduced cell viability by 42%, establishing its toxicity to retinal neuron cells. Risuteganib pre-treatment demonstrated protective effect by reducing the loss of cell viability from 42% to 18%.

Conclusion: Risuteganib alone did not alter cell viability, while pre-treatment demonstrated measurable protection against kainic acid-based cytotoxicity in primary mouse retinal neuron cells.

### Cytoprotective Effects in Human RPE Cells (ARPE-19)

Purpose: Investigate cytoprotective properties of risuteganib in human RPE cells (ARPE-19) exposed to hydrogen peroxide, which is a reactive oxygen species that can induce cell death at elevated levels. Methods: ARPE-19 cells were cultured in laminin-coated trans-wells for 2 weeks to induce differentiation. Cells were then exposed to the experimental conditions: (1) untreated control, (2) 1.0 mg/mL risuteganib, (3) 100 μM hydrogen peroxide (H₂O₂), and (4) 1.0 mg/mL risuteganib for 24 hours before 100 μM H₂O₂ exposure. 8 hours after H₂O₂ treatment, dead and live cell numbers were measured using Trypan blue exclusion assay on a hemocytometer.

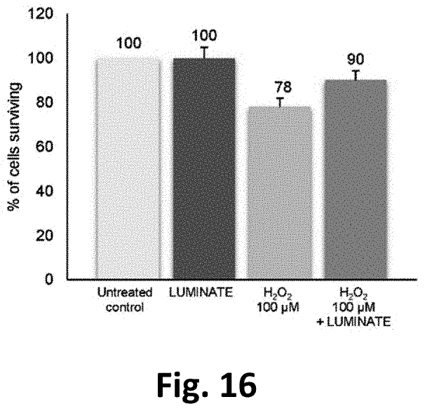

Results/Discussion: As shown graphically in FIG. 16, risuteganib treatment alone did not induce detectable change in cell viability, while H₂O₂ treatment moderately reduced cell viability by 22%. Risuteganib pre-treatment demonstrated protective effect by reducing the loss of cell viability from 22% to 10%.

Conclusion: Risuteganib alone did not alter cell viability, while pre-treatment demonstrated measurable protection against H₂O₂-based cytotoxicity in human RPE cells.

### Cytoprotective Effects of Risuteganib and Various Anti-VEGF Agents in Human (MIO M1) Müller Cells

Purpose: To determine the effects of risuteganib and anti-VEGF drugs on the cell viability of cultured human retinal Müller cells (MIO-M1).

Methods: The immortalized human retinal Müller cell line (MIO-M1) was obtained from the Department of Cell Biology of the University College, London. Cells were cultured in Dulbecco's Modified Eagle's Medium (DMEM) and plated in 96-well plates for 24 hours before treatment with 0.5×, 1× or 2× concentrations of 1 mg/50 μL risuteganib, or 1× of ranibizumab, bevacizumab or aflibercept. Dosage was based on clinical dose of each compound. The experiments were repeated 3 times with 7-8 replicates each. After 24 hours of drug treatment, MTT NAD(P)H-dependent colorimetric assay was used to assess the number of viable cells present in the cultures. Absorbance ratios were normalized to untreated control as 100%. Statistical analysis was performed in GraphPad Prism software program.

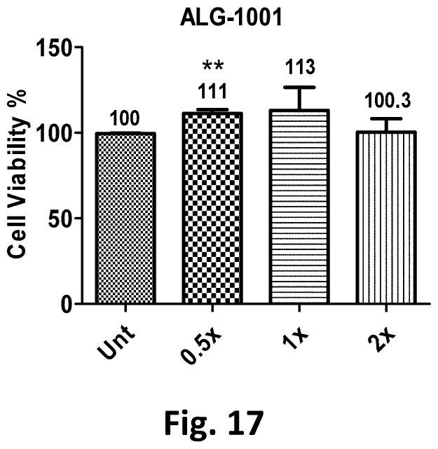

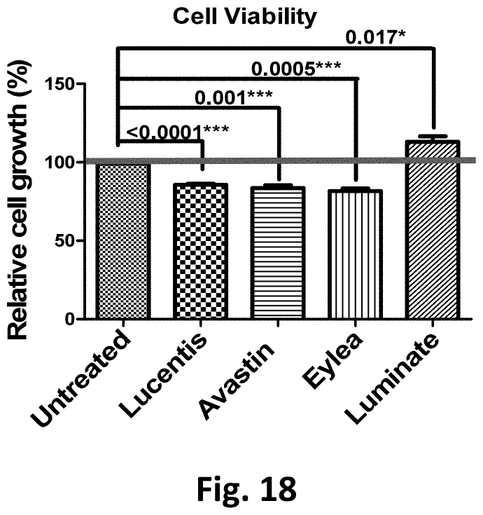

Results/Discussion: As shown graphically in FIG. 17, MIO-M1 cells treated with 0.5× risuteganib showed increased cell viability compared to the untreated cultures (111.3±2.189 versus 100±0.29, p=0.0058). The MIO-M1 cultures treated with 1×(113.5±13.5, p=0.37) and 2×(100.3±7.8, p=0.92) risuteganib showed similar levels of cell viability to the untreated MIO-M1 cultures. This is in contrast to experiments showing decreased cell viability in MIO-M1 cells treated with 1× concentration of ranibizumab (Lucentis®), bevacizumab (Avastin®), and aflibercept (Eylea®) as summarized graphically in FIG. 18.

Conclusion: Risuteganib treatments either significantly increased or did not change MIO-M1 cell viability in comparison to untreated controls, while anti-VEGF drugs significantly reduced cell viability.

### Effects of Risuteganib and Various Anti-VRGF Agents on Reactive Oxygen Species Levels in Human (MIO-M1) Müller Cells

Purpose: To determine the effects of risuteganib and anti-VEGF drugs on reactive oxygen species (ROS) levels in cultured human retinal Müller cells (MIO-M1). Elevated ROS levels can disrupt normal cellular functions, leading to reduced cell health and possible cell death.

Methods: The immortalized human retinal Müller cell line (MIO-M1) was obtained from the Department of Cell Biology of the University College, London. Cells were cultured in Dulbecco's Modified Eagle's Medium (DMEM) and plated in 24-well plates for 24 hours before treatment with 1× concentration of 1 mg/50 μL ALG-1001, ranibizumab, bevacizumab, or aflibercept. Dosage was based on clinical dose of each compound. The experiments were repeated 3 times with 6 replicates each. After 24 hours drug treatment, ROS level was measured using the fluorescent dye 2′,7′-dichlorodihydrofluorescein diacetate. The signals were read using the Biotek Synergy HT plate reader with EX filter in 482 nm and EM filter in 520 nm. Results were normalized to untreated control as 100%. Statistical analysis was performed in GraphPad Prism software program.

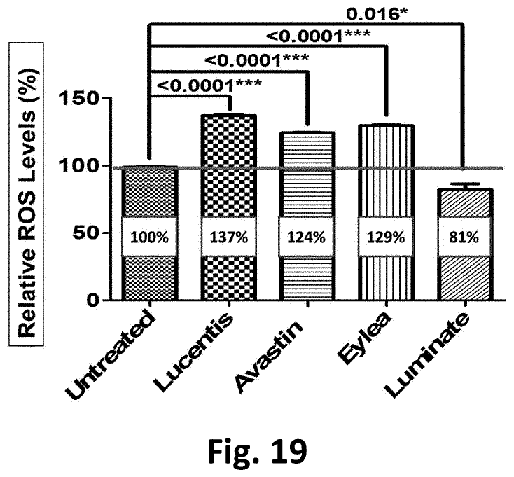

Results/Discussion: As shown graphically in FIG. 19, MIO-M1 cells treated with 1× of risuteganib showed statistically significant reduced levels of ROS compared to the untreated control cultures (−19%, p=0.016). In comparison, 1× of anti-VEGF drugs significantly increased ROS levels by 37% (Lucentis®), 24% (Avastin®), and 29% (Eylea®).

Conclusion: Risutiganib treatment significantly reduced MIO-M1 ROS levels in comparison to untreated controls, while anti-VEGF drugs significantly increased ROS levels.

### Effects of Risuteganib and Various Anti-VRGF Agents on Mitochondrial Membrane Potential (ΔΨm) in Human (MIO-M1) Müller Cells

Purpose: To determine the effects of risuteganib on the mitochondrial membrane potential (ΔΨm) in cultured human retinal Müller cells (MIO-M1). Loss of ΔΨm is a marker for early cell death.

Methods: The immortalized human retinal Müller cell line (MIO-M1) was obtained from the Department of Cell Biology of the University College, London. Cells were cultured in Dulbecco's Modified Eagle's Medium (DMEM) and plated in 24-well plates for 24 hours before treatment with 1× concentration of 1 mg/50 μL risuteganib, ranibizumab, bevacizumab, or aflibercept. Dosage was based on clinical dose of each compound. The experiments were repeated 3 times with 6 replicates each. After 24 hours drug treatment, the ΔΨm was measured using the JC-1 kit, a cationic dye that fluoresces red within the mitochondria of healthy, live cells. In the stressed or apoptotic cells, the mitochondrial membrane potential collapses and the cationic dye fluoresces green. First, cells were rinsed with fresh media and then incubated with the JC-1 reagent for 15 minutes at 37 degrees C. The dyes were then removed, and phosphate buffered saline was added to each well. The Red fluorescence (live cells) was read at EX 550 nm and EM 600 nm. The Green fluorescence (apoptotic cells) was read at EX 483 nm and EM 535 nm. The changes in ΔΨm were calculated by the ratio of red to green fluorescence. Results were normalized to untreated control as 100%. Statistical analysis was performed in GraphPad Prism software program.

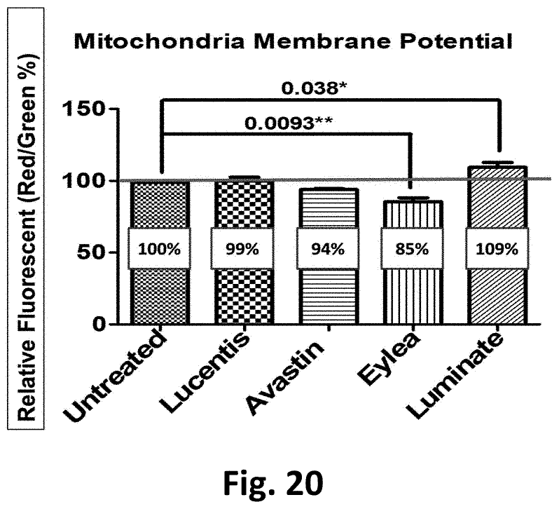

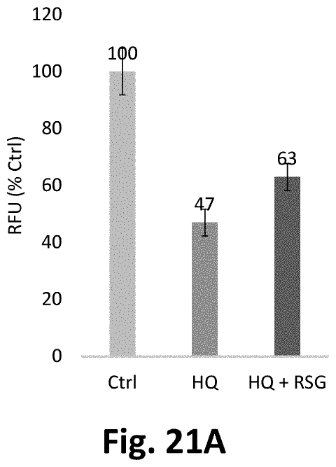

Results/Discussion: As summarized graphically in FIG. 20, MIO-M1 cells treated with 1× risuteganib (109.3±4.91, p=0.038) showed elevated mitochondrial membrane potential compared to the untreated control cultures. This is in contrast to decreased ΔΨm in MIO-M1 cells treated with 1× aflibercept (Eylea®, p=0.0093). The other anti-VEGF agents tested did not cause significant changes mitochondrial membrane potential compared to the untreated control cultures. An elevated mitochondrial membrane potential is believed to correlate with improved cellular function of the mitochondria.

Conclusion: Risuteganib treatment significantly increased MIO-M1 mitochondria membrane potential in comparison to untreated controls, while Eylea® significantly reduced mitochondria membrane potential.

### Regulation of Reactive Oxygen Species Levels, Mitochondrial Membrane Potential, and Cell Viability in Primary Human RPE Cells

Purpose: To determine if risuteganib protects against hydroquinone (HQ)-mediated cell injury, elevated ROS level and reduced mitochondrial membrane potential (Δψm) in cultured human RPE cells. Elevated ROS levels increase oxidative stress in the cells, leading to reduced cell health and cell death. Loss of ΔΨm is a marker for early cell death.

Methods: Primary human RPE cells were seeded on collagen-coated 96-well plates in triplicates at 8K, 10K and 17K cells/well, respectively. Cells reached 80% to 100% confluence 24 hours after plating, and confluent cells were then grown for an additional 4 or 5 days until growth was density arrested. On day 6 after plating, cells in the plate upper half were loaded with 20 μM CM-H2DCFDA (measures ROS level) and in the plate lower half with 10 μM JC-1 (measures Δψm) for 30 minutes at 37° C. Cells were washed twice with in media and treated with HQ at dosages between 125-180 uM in the presence or absence of 0.4 mM risuteganib for 3-4 hours. For the ROS and Δψm assays, a fluorescence plate reader was used to quantify ROS production (490-nm excitation, 522-nm emission), and green monomer of JC-1 (490-nm excitation, 522-nm emission) and red JC-1 aggregate (535-nm excitation, 590-nm emission), respectively. For the WST-1 assay, 4 hours or 5 hours after treatment, the media were removed, and fresh media were added into cells and incubated for 20 minutes at 37° C. with WST-1 solution. The WST reagent was quantified with a plate reader at 440 nm and a reference wavelength at 690 nm. Data were normalized to untreated control as 100% and were expressed as the mean±SD. Student's t-test was used to determine whether there were statistically significantly differences between treatment groups.

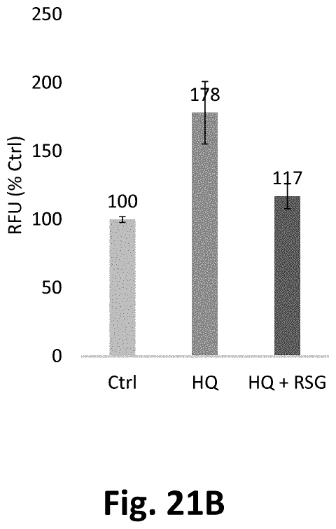

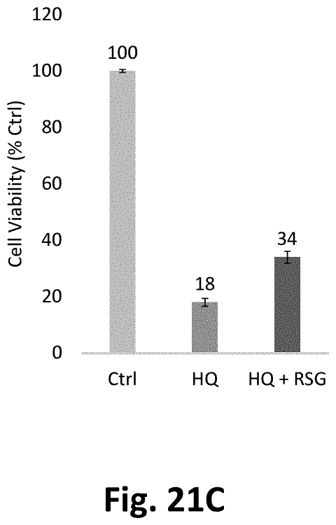

Results/Discussion: The results of this study are summarized graphically in FIGS. 21A, 21B and 21C. Compared to untreated cells, HQ exposure significantly decreased Δψm (−53%) (FIG. 21A) and cell viability (−82%) (FIG. 21C) but increased ROS levels (78%) (FIG. 21B). Risuteganib co-treatment significantly improved HQ-reduced Δψm (16% improvement) (FIG. 21A) and cell viability (16% improvement) (FIG. 21C), while suppressed HQ-induced ROS production (61% reduction) (FIG. 21B). The assays were repeated in RPE cells from 3 different donor and similar results were observed.

Conclusion: risuteganib moderated hydroquinone-induced ROS level elevation, Δψm reduction, and protected against hydroquinone-mediated human RPE cell injury.

### Other Peptides Expected to have Effects Comparable to Risuteganib

The effects and mechanisms of action referred to in this patent application are not necessarily limited to Risuteganib. Other peptides, including those described in the above-incorporated U.S. Pat. Nos. 9,018,352; 9,872,886; 9,896,480 and 10,307,460 and in United States Patent Application Publication Nos. 2018/0207227 and 2019/0062371, which may reasonably be expected to also exhibit the herein described effects and/or mechanisms of action. Specific examples of other peptides believed to exhibit some or all of these effects or mechanisms include, but are not necessarily limited to, comprise peptides that consist of or include an amino acid sequence having the formula:

Y—X—Z
wherein:
   Y=R, H, K, Cys(acid), G or D;
X=G, A, Cys(acid), R, G, D or E; and
   Z=Cys(acid), G, C, R, D, N or E.

Also, such peptides may comprise or consist of the amino acid sequences; R-G-Cys(Acid), R—R-Cys, R-CysAcid)-G, Cys(Acid)-R-G, Cys(Acid)-G-R, R-G-D, R-G-Cys(Acid). H-G-Cys(Acid), R-G-N, D-G-R, R-D-G, R-A-E, K-G-D, R-G-Cys(Acid)-G-G-G-D-G, Cyclo-{R-G-Cys(acid)-F—N-Me-V}, R-A-Cys (Acid), R-G-C, K-G-D, Cys(acid)-R-G, Cys(Acid)-G-R, Cyclo-{R-G-D-D-F—NMe-V}, H-G-Cys(acid) and salts thereof. Possible salts include but are not limited to acetate, trifluoroacetate (TFA) and hydrochloride salts. Such peptides are useful at least for inhibiting neovascularization of the development of pathological or aberrant blood vessels in human or animal subjects. Examples of such peptides, along with indications of their respective levels of activity in suppressing retinal neovascularization in mice, are shown in Table 27 of the above-incorporated United States Patent Application Publication No. 2019/0062371, which is reproduced below:

TABLE 27
ADDITIONAL PEPTIDES

| | | Mean % Reduction | |
|---|---|---|---|
| Test | | of Retinal | Activity |
| Compound | | Neovascularization | At Dose |
| Number | Test Compound | In ROP Model | Tested |
| 1 | R - G - Cys(acid)•TFA - | 61 | Active |
| | ROP | | |
| 1(CNV) | R- G - Cys(acid)•TFA - | 49 - FIG. 11 | Active |
| | CNV | | |
| 2(CNV) | R- G - Cys(acid)•Acetate - | 56 - FIG. 11 | Active |
| | CNV | | |
| 2 | R- G - Cys(acid)•Acetate - | 72 | Active |
| | ROP | | |
| 3 | R - A - Cys (acid)•TFA | 60 | Active |
| 4 | R - G - Cysteine•TFA | 66 | Active |
| 5 | R - Cys(acid) - G•TFA | 33 | Slightly |
| | | | Active |
| 6 | K - G - Cys (acid)•TFA | 0 | Not Active |
| 7 | R - G - Cys(acid)-G-G-G- | 62 | Active |
| | D-G•TFA | | |
| 8 | Cys(acid) - R - G•TFA | 21 | Slightly |
| | | | Active |
| 9 | Cys(acid) - G - R•TFA | 63 | Active |
| 10 | Cys(acid) - A - R•TFA | 0 | Not Active |
| 11 | G - Cys(acid) - R•TFA | 0 | Not Active |
| 12 | Cyclo-{R-G-Cys(acid)- | 57 | Active |
| | F—N—Me—V} Acetate | | |
| 13 | Cyclo-{R-G-D-D-F-NMe- | 75 | Active |
| | V}•TFA | | |
| 14 | H - G -Cys(acid)•TFA | 28 | Slightly |
| | | | Active |
| 15 | R - G - D•TFA | 37 | Slightly |
| | | | Active |
| 16 | R - G - N•TFA | 64 | Active |
| 17 | D - G - R•TFA | 56 | Active |
| 18 | R - D - G•TFA | 44 | Active |
| 19 | R - A - E•TFA | 63 | Active |
| 20 | K - G - D•TFA | 40 | Active |
| 21 | R - G - E•TFA | 0 | Not Active |
| 22 | R - E - G•TFA | 0 | Not Active |
| 23 | R - A - D•TFA | 0 | Not Active |
| 24 | R-G-Cys(acid)•TFA + | 58 | Active |
| | Taurine | | |
| 25 | Taurine | 33 | Slightly |
| | | | Active |

Additional examples of other potentially useable peptides include, but are not necessarily limited to, those described along with risuteganib (ALG-1001) in the above-incorporated U.S. Pat. Nos. 9,018,352; 9,872,886; 9,896,480 and 10,307,460. These include peptides which comprise Glycinyl-Arginyl-Glycinyl-Cysteic Acid-Threonyl-Proline-COOH or which have the formula:

X1-Arg-Gly-Cysteic Acid-X
where X and X1 are independently selected from: Phe-Val-Ala, -Phe-Leu-Ala, -Phe-Val-Gly, -Phe-Leu-Gly, -Phe-Pro-Gly, -Phe-Pro-Ala, -Phe-Val; or from Arg, Gly, Cysteic, Phe, Val, Ala, Leu, Pro, Thr and salts, combinations, D-isomers and L-isomers thereof.

It is to be appreciated that, although this patent application contains specific examples of studies wherein the anti-integrin peptide is administered by intravitreal injection, it is to be appreciated that any alternative effective route of administration including but not limited to topical and systemic routes (e.g., eye drops, oral, intravenous, intramuscular, subcutaneous, intranasal, buccal, transdermal, etc.) or by release from a suitable drug delivery implant substance or device. Additionally, although the above includes reference to certain examples or embodiments, various additions, deletions, alterations and modifications may be made to those described examples and embodiments without departing from the intended spirit and scope of this disclosure. For example, any elements, steps, members, components, compositions, reactants, parts or portions of one embodiment or example may be incorporated into or used with another embodiment or example, unless otherwise specified or unless doing so would render that embodiment or example unsuitable for its intended use. Also, where the steps of a method or process have been described or listed in a particular order, the order of such steps may be changed unless otherwise specified or unless doing so would render the method or process unsuitable for its intended purpose. Additionally, the elements, steps, members, components, compositions, reactants, parts or portions of any invention or example described herein may optionally exist or be utilized in the absence or substantial absence of any other element, step, member, component, composition, reactant, part or portion unless otherwise noted. All reasonable additions, deletions, modifications and alterations are to be considered equivalents of the described examples and embodiments and are to be included within the scope of the following claims.

What is claimed is:

1. A method for a) improving best corrected visual acuity of an eye of a subject suffering from non-exudative age related macular degeneration and/or b) improving color vision in an eye of a subject suffering from impaired color vision, said method comprising the step of administering to the subject an anti-integrin peptide in an amount which is effective to improve best corrected visual acuity and/or color vision in said eye.

2. A method according to claim 1 wherein the peptide is linear or cyclic and comprises Glycinyl-Arginyl-Glycinyl-Cysteic Acid-Threonyl-Proline-COOH or a fragment, congener, derivative, pharmaceutically acceptable salt, hydrate, isomer, multimer, cyclic form, linear form, conjugate, derivative or other modified form thereof.

3. A method according to claim 2 wherein the peptide comprises risuteganib.

4. A method according to claim 1 wherein the peptide has the formula:
X1-R-G-Cysteic Acid-X
where X and X1 are independently selected from: Phe-Val-Ala, -Phe-Leu-Ala, -Phe-Val-Gly, -Phe-Leu-Gly, -Phe-Pro-Gly, -Phe-Pro-Ala, -Phe-Val; or from Arg, Gly, Cysteic, Phe, Val, Ala, Leu, Pro, Thr and salts, combinations, D-isomers and L-isomers thereof.

5. A method according to claim 1 wherein the peptide has the general formula:
Y—X—Z
wherein:
Y=R, H, K, Cys(acid), G or D;
X=G, A, Cys(acid), R, G, D or E; and
Z=Cys(acid), G, C, R, D, N or E.

6. A method according to claim 1 wherein the peptide comprises or consists of an amino acid sequence selected from: R-G-Cys(Acid), R—R-Cys, R-CysAcid)-G, Cys(Acid)-R-G, Cys(Acid)-G-R, R-G-D, R-G-Cys(Acid). H-G-Cys(Acid), R-G-N, D-G-R, R-D-G, R-A-E, K-G-D, R-G-Cys(Acid)-G-G-G-D-G, Cyclo-{R-G-Cys(acid)-F—N-Me-V}, R-A-Cys (Acid), R-G-C, K-G-D, Cys(acid)-R-G, Cys(Acid)-G-R, Cyclo-{R-G-D-D-F—NMe-V}, H-G-Cys(acid) and salts thereof.

7. A method according to claim 1 wherein the peptide is administered intraviterally.

8. A method according to claim 7 wherein the peptide comprises risuteganib and wherein dose in the range of from 0.01 mg risuteganib to 10.0 mg risuteganib is administered intravitreally.

9. A method according to claim 7 wherein the peptide comprises risuteganib and wherein dose in the range of from 0.05 mg risuteganib to 5.0 mg risuteganib is administered intravitreally.

10. A method according to claim 7 wherein the peptide comprises risuteganib and wherein from 1 mg to 1.5 mg of risuteganib is administered intravitreally.

11. A method according to claim 1 wherein the peptide is administered only once.

12. A method according to claim 1 wherein the peptide is administered a plurality of times.

13. A method according to claim 12 wherein an interval of from 1 week to 20 weeks exists between administrations of the peptide.

14. A method according to claim 12 wherein an interval of from 12 week to 16 weeks exists between administrations of the peptide.

15. A method according to claim 14 wherein the peptide comprises risuteganib and wherein each intravitreal administration of the peptide delivers a dose of 1 mg. to 1.5 mg risuteganib.

16. A method according to claim 1 wherein the anti-integrin peptide causes downregulation of integrin αMβ2.

17. A method according to claim 1 wherein the anti-integrin peptide reduces expression of a complement 3 receptor.
